(19) United States
(12) Patent Application Publication
(10) Pub. No.: US 2026/0166144 A1
(43) Pub. Date: Jun. 18, 2026

(54) CHIMERIC POLYPEPTIDE AND USE THEREOF

(71) Applicant: CARSGEN LIFE SCIENCES CO., LTD., Shanghai (CN)

(72) Inventors: Zonghai LI, Shanghai (CN); Hua JIANG, Shanghai (CN); Peng WANG, Shanghai (CN); Honghong ZHANG, Shanghai (CN)

(73) Assignee: CARSGEN LIFE SCIENCES CO., LTD., Shanghai (CN)

(21) Appl. No.: 18/854,214

(22) PCT Filed: Apr. 7, 2023
(86) PCT No.: PCT/CN2023/086980

§ 371 Date: Oct. 4, 2024

(30) Foreign Application Priority Data
Apr. 7, 2022 (CN) 202210361286.0
Apr. 15, 2022 (CN) 202210402132.1

(51) Int. Cl. A61K 40/31

(52) U.S. Cl. CPC A61K 40/31; C07K 2317/565

(57) ABSTRACT

Provided are an antibody/T cell receptor chimera carrying different signal peptides and the use thereof. The provided chimeric polypeptide contains a chain A and a chain B, wherein the chain A contains a first antigen binding domain and a first constant region, and the chain B contains a second antigen binding domain and a second constant region: (i) the first and/or second antigen binding domain is a heavy chain or heavy chain variable region of an antibody, which is operably linked to a heavy chain signal peptide or a TCR subunit signal peptide; and (ii) the first and/or second antigen binding domain is a light chain or light chain variable region of an antibody, which is operably linked to a light chain signal peptide or a TCR subunit signal peptide.

### RELATED APPLICATIONS

This patent application claims priority to the Chinese patent application with the application number of 2022103612860 filed on Apr. 7, 2022, and the Chinese patent application with the application number of 2022104021321 filed on Apr. 15, 2022.

### SEQUENCE LISTING FILE SUBMITTED AT THE SAME TIME

The entire contents of the following XML file are incorporated herein by reference in their entirety: sequence listing in computer readable format (CRF) (name: FG00772PCT-sequence listing.xml, date: 20230407, size: 68 KB).

### TECHNICAL FIELD

The present application belongs to the field of immunotherapy. More specifically, the present application relates to engineered cells comprising recombinant TCR receptors and uses thereof.

### BACKGROUND

Therapy with chimeric antigen receptor-expressing T cells (CAR-T) and therapy with exogenous T cell receptor-expressing T cells (TCR-T) have shown significant clinical effects in tumor immunotherapy. Compared with CAR-T therapy, TCR-T therapy has lower toxic and side effects; the antigen abundance required for TCR-T activation is much lower than that required for CAR-T activation; in some solid tumors where CAR-T therapy is ineffective, TCR-T may have better anti-tumor effect. Therefore, TCR-T has great prospects in treating cancer.

One challenge faced in the application of TCR-T is that the endogenous TCR α/β chain of T cells can be mismatched with the introduced exogenous TCR α/β chain, which reduces the expression of the introduced exogenous TCRs on the cell surface. The mismatched TCRs competitively bind to CD3 with the exogenous TCR α/β chain dimers, resulting in a large number of TCRs without tumor antigen targeting. Reducing the mismatch between endogenous TCR subunits and exogenous TCR subunits and increasing the distribution of exogenous recombinant TCRs on the cell surface are important strategies to improve the safety and effectiveness of TCR-T therapy.

### SUMMARY

The present application involves the following contents:
(1). A T cell receptor (TCR) chimera (ATC), comprising an antigen-binding domain and a TCR subunit constant region; and the signal peptide connected with the antigen-binding domain comprises a TCR signal peptide, a GMCSF signal peptide, an IgG signal peptide or a combination thereof.
(2). The ATC according to (1), wherein the antigen-binding domain comprises one or two immunoglobulin variable regions.
(3). The ATC according to (1) or (2), wherein the antigen-binding domain comprises heavy chain variable region(s) (VH) and light chain variable region(s) (VL) of an antibody.
(4). The ATC according to (3), wherein the VH and/or VL are connected with the TCR signal peptide; or the VH and/or VL are connected with the GMCSF signal peptide; or the VH and/or VL are connected with the IgG signal peptide.
(5). The ATC according to (4), wherein the VH and VL are connected with a signal peptide IgGsL1, respectively; or the VH and VL are connected with a signal peptide IgGsH1, respectively; or the VH is connected with a signal peptide IgGsL1, and the VL is connected with a signal peptide IgGsH1; or the VH is connected with a signal peptide IgGsH1, and the VL is connected with a signal peptide IgGsL1; or the VH is connected with a TRAV signal peptide, and the VL is connected with a TRBV signal peptide; or the VH is connected with a TRBV signal peptide, and the VL is connected with a TRAV signal peptide.
(6). The ATC according to any one of (1)-(5), wherein the sequence of the TRAV signal peptide is as shown in SEQ ID NO: 1, and the sequence of the TRBV signal peptide is as shown in SEQ ID NO: 3, the sequence of the GMCSF signal peptide is as shown in SEQ ID NO:5, the sequence of the IgGsL1 signal peptide is as shown in SEQ ID NO:9, and the sequence of the IgGsH1 signal peptide is as shown in SEQ ID NO:14.
(7). The ATC according to any one of (1)-(6), wherein the TCR subunit constant region comprises a natural and/or modified TRAC peptide and/or TRBC peptide.
(8). The ATC according to any one of (1)-(7), wherein the TRAC peptide is connected with the heavy chain variable region (VH), and the TRBC peptide is connected with the light chain variable region (VL); the TRAC peptide is connected with the light chain variable region (VH), and the TRBC peptide is connected with the heavy chain variable region (VL).
(9). The ATC according to any one of (1)-(8), wherein the ATC can associate with the CD3ζ polypeptide.
(10). The ATC according to any one of (1)-(9), wherein the ATC can activate the CD3ζ polypeptide associated with the ATC after binding to an antigen.
(11). The ATC according to any one of (1)-(10), wherein activation of the CD3ζ polypeptide can activate immune effector cells.
(12). The ATC according to any one of (1)-(11), wherein the ATC binds to a tumor antigen.
(13). The ATC according to (12), wherein the tumor antigen is selected from the group consisting of: GPC3, EGFR, Claudin18.2, BCMA, mesothelin, and CD19.
(14). The ATC according to any one of (1)-(13), wherein the ATC comprises a TCR subunit constant region with synonymously mutated nucleotide sequence.
(15). The ATC according to any one of (1)-(14), wherein the ATC comprises the nucleotide sequence of SEQ ID NO: 25 or 32, or the amino acid sequence of SEQ ID NO: 19, 21, 23 or 24.
(16). An immune effector cell, comprising the ATC according to any one of (1)-(15).
(17). The cell according to (16), wherein the expression, activity and/or signaling of endogenous TCR subunits of the cell is reduced or inhibited.
(18). The cell according to (16) or (17), wherein the endogenous TCR expression is reduced or inhibited by using gene knockout technology and/or gene silencing technology, preferably, using TALEN nuclease, meganuclease, zinc finger nuclease, CRISPR/Cas9, Argonaute or a combination thereof.
(19). The cell according to any one of (16)-(18), wherein the ATC does not comprise nucleic acid sequences targeted by the gene knockout technology and/or gene silencing technology.
(20). The cell according to (19), wherein the ATC does not comprise gRNA target sequence.
(21). The cell according to any one of (16)-(20), wherein the ATC nucleic acid molecule comprises a nucleic acid molecule that is no longer the target sequence targeted by the gene knockout technology and/or gene silencing technology after synonymous mutation of the nucleotide(s).
(22). The cell according to any one of (16)-(21), wherein the gRNA target sequence comprises the sequence of SEQ ID NO: 49 and/or SEQ ID NO: 50.
(23). The cell according to any one of (20)-(22), wherein the cell is selected from the group consisting of: T cells, cytotoxic T lymphocytes (CTL), regulatory T cells, NK cells, natural killer T cells (NKT), human embryonic stem cells, and pluripotent stem cells from which lymphoid cells can be differentiated.
(24). The cell according to any one of (16)-(23), wherein the cell is an autologous or allogeneic cell.
(25). A pharmaceutical composition comprising an effective amount of the immune effector cell according to any one of (16)-(24) and pharmaceutically acceptable excipients.
(26). The pharmaceutical composition according to (25), which is used to treat tumors.
(27). A method for reducing tumor burden in a subject, comprising administering to the subject an effective amount of the immune effector cell according to any one of (16)-(24) or the pharmaceutical composition according to (25) or (26).
(28). A method for treating or preventing tumors, including administering to a subject an effective amount of the immune effector cell according to any one of (16)-(24) or the pharmaceutical composition according to (25) or (26).
(29). The method according to (28), wherein the tumor is selected from the group consisting of: liver cancer, lung cancer, breast cancer, ovarian cancer, kidney cancer, thyroid cancer, gastric cancer, colorectal cancer, pancreatic cancer, multiple myeloma tumors, and hematological tumors.
(30). The method according to (28) or (29), wherein the tumor is a GPC3-positive tumor.
(31). A method for producing antigen-specific immune effector cells, including introducing a nucleic acid molecule encoding the ATC according to any one of (1)-(15) into the immune effector cells.
(32). The method according to (31), further including introducing a gRNA into the immune effector cells.
(33). The method according to (31) or (32), wherein the nucleic acid molecule is comprised in a vector.
(34). A method for prolonging survival of a subject suffering from a tumor, including administering to the subject an effective amount of the immune effector cell according to any one of (16)-(24) or the pharmaceutical composition according to (25) or (26).
(35). A polynucleotide encoding the ATC according to any one of (1)-(15).
(36). A nucleic acid composition comprising the ATC according to any one of (1)-(15).
(37). The nucleic acid composition according to (36), wherein the nucleic acid molecule is comprised in a vector.
(38). A vector comprising the nucleic acid composition according to (36) or (37).
(39). A kit comprising the ATC according to any one of (1)-(15), the immune effector cell according to any one of (16)-(24), the pharmaceutical composition according to (25) or (26), the nucleic acid composition according to (36) or (37), or the vector according to (38).
(40). The kit according to (39), further comprising written instructions for treating and/or preventing tumors, pathogenic infections, autoimmune diseases, or allogeneic transplantation.
(41). Use of any of the ATC according to any one of (1)-(15) in treatment.
(42). Use of the pharmaceutical composition according to (25) or (26) in treatment.
(43). Use of the immune effector cell according to any one of (16)-(24) in treatment.
(44). Use of the immune effector cell according to any one of (16)-(24) or the pharmaceutical composition according to (25) or (26) for reducing the tumor burden in a subject.
(45). Use of the immune effector cell according to any one of (16)-(24) or the pharmaceutical composition according to (25) or (26) for treating or preventing tumors.
(46). Use of the immune effector cell according to any one of (16)-(24) or the pharmaceutical composition according to (25) or (26) for prolonging survival of a subject suffering from a tumor.

The present application also involves the following contents:
1. A T cell receptor (TCR) chimera (ATC), comprising an antigen-binding domain and a TCR subunit constant region; the signal peptide connected with the antigen-binding domain is selected from the group consisting of: a TRAV signal peptide, a TRBV signal peptide, a GMCSF signal peptide, an IgGsL1 and an IgGsH1 signal peptide.
2. The ATC according to item 1, wherein the antigen-binding domain comprises the heavy chain variable region (VH) and the light chain variable region (VL) of an antibody.
3. The ATC according to item 1 or 2, wherein the VH and VL are connected with the GMCSF signal peptide, respectively; or the VH is connected with the signal peptide IgGsL1, and the VL is connected with the signal peptide IgGsH1; or the VH is connected with the signal peptide IgGsH1, and the VL is connected with the signal peptide IgGsL1; or the VH is connected with the TRAV signal peptide, and the VL is connected with the TRBV signal peptide; or the VH is connected with the TRBV signal peptide, and the VL is connected with the TRAV signal peptide.
4. The ATC according to any one of items 1-3, wherein the sequence of the TRAV signal peptide is as shown in SEQ ID NO: 1, the sequence of the TRBV signal peptide is as shown in SEQ ID NO: 3, the sequence of the GMCSF signal peptide is as shown in SEQ ID NO: 5, the sequence of the IgGsL1 signal peptide is as shown in SEQ ID NO: 9, and the sequence of the IgGsH1 signal peptide is as shown in SEQ ID NO: 14.
5. The ATC according to any one of items 1-4, wherein the antigen-binding domain of the ATC is connected with the signal peptide directly or through a linker.
6. The ATC according to any one of items 1-5, wherein the ATC comprises a natural and/or modified TRAC peptide and a TRBC peptide; or the ATC comprises a natural and/or modified TRGC peptide and a TRDC peptide.
7. The ATC according to item 6, wherein the TRAC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 19; the TRBC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 21; the TRGC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid of SEQ ID NO: 23; and the TRDC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 24.
8. The ATC according to any one of items 1-7, wherein the ATC binds to a tumor antigens and/or a pathogen antigen.
9. The ATC according to item 8, wherein the tumor antigen is selected from the group consisting of: GPC3, EGFR, Claudin18.2, BCMA, mesothelin, and CD19.
10. The ATC according to any one of items 1-9, wherein the antigen-binding domain comprises VL of an antibody that recognizes GPC3, and the amino acid sequence of the VL is as shown in SEQ ID NO: 39 or has 70-100% sequence identity thereto; and/or comprises

VH of an antibody that recognizes GPC3, and the amino acid sequence of the VH is as shown in SEQ ID NO: 38 or has 70-100% sequence identity thereto.
11. The ATC according to any one of items 1-10, wherein the ATC comprises a nucleotide sequence of SEQ ID NO: 25, 32, 20, 22, 25 or 32, or comprises the amino acid sequences of SEQ ID NO: 19 or 21.
12. An immune effector cell comprising the ATC according to any one of items 1-11.
13. The cell according to item 12, wherein the expression, activity and/or signaling of endogenous TCR subunits of the cell is reduced or inhibited.
14. The cell according to item 12 or 13, wherein the expression of the endogenous TCR subunits is reduced or inhibited by using gene knockout technology and/or gene silencing technology selected from the group consisting of: TALE nuclease, meganuclease, zinc finger nuclease, CRISPR/Cas9, Argonaute, guided editing technology, meganuclease technology or a combination thereof.
15. The cell according to any one of items 12-14, wherein the ATC does not comprise nucleic acid sequences targeted by gene knockout technology and/or gene silencing technology.
16. The cell according to any one of items 12-15, wherein the nucleic acid molecule of the ATC comprises a nucleic acid molecule that is no longer the target sequence targeted by gene knockout technology and/or gene silencing technology after synonymous mutation of the nucleotide(s).
17. The cell according to any one of items 12-16, wherein the cell comprises gRNA, the sequence of which is as shown in SEQ ID NO: 49 or 50.
18. The cell according to any one of items 12-17, wherein the cell is selected from the group consisting of: T cells, cytotoxic T lymphocytes (CTL), regulatory T cells, NK cells, natural killer T cells (NKT), human embryonic stem cells, and pluripotent stem cells from which lymphoid cells can be differentiated.
19. The cell according to any one of items 12-18, wherein the cell is an autologous or allogeneic cell.
20. A pharmaceutical composition comprising an effective amount of the immune effector cell according to any one of items 12-19 and pharmaceutically acceptable excipients.
21. The pharmaceutical composition according to item 20, which is used to treat tumors.
22. A method for reducing tumor burden in a subject, including administering to the subject an effective amount of the immune effector cell according to any one of items 12-19 or the pharmaceutical composition according to item 20 or 21.
23. A method for treating or preventing a tumor, including administering to the subject an effective amount of the immune effector cell according to any one of items 12-19 or the pharmaceutical composition according to item 20 or 21.
24. The method according to item 23, wherein the tumor is selected from the group consisting of: liver cancer, lung cancer, breast cancer, ovarian cancer, kidney cancer, thyroid cancer, gastric cancer, colorectal cancer, pancreatic cancer, multiple myeloma, and hematological malignancy.
25. The method according to any one of items 22-24, wherein the tumor is a GPC3-positive tumor.
26. A method for producing antigen-specific immune effector cells, including introducing a nucleic acid molecule encoding the ATC according to any one of items 1-11 into the immune effector cell.
27. The method according to item 26, further including introducing a gRNA targeting endogenous TCR into the immune effector cell.
28. A method for prolonging survival of a subject suffering from a tumor, including administering to the subject an effective amount of the immune effector cell according to any one of items 12-19 or the pharmaceutical composition according to item 20 or 21.
29. A polynucleotide encoding the ATC according to any one of items 1-11.
30. A vector comprising the polynucleotide according to item 29.
31. A kit comprising the ATC according to any one of items 1-11, the immune effector cell according to any one of items 12-19, the pharmaceutical composition according to any one of item 20 or 21, and the polynucleotide according to item 29, or the vector according to item 30.
32. The kit according to item 31, further comprising written instructions for treating and/or preventing tumors, pathogenic infections, autoimmune diseases, or allogeneic transplantation.
33. Use of the ATC according to any one of items 1-11 in treating diseases.
34. Use of the pharmaceutical composition according to item 20 or 21 in treating diseases.
35. Use of the immune effector cell according to any one of items 12-19 in treating diseases.
36. Use of the immune effector cell according to any one of items 12-19 or the pharmaceutical composition according to item 20 or 21 for reducing tumor burden in a subject.
37. Use of the immune effector cell according to any one of items 12-19 or the pharmaceutical composition according to item 20 or 21 for treating or preventing tumors.
38. Use of the immune effector cell according to any one of items 12-19 or the pharmaceutical composition according to item 20 or 21 for prolonging survival of a subject suffering from a tumor.

It should be understood that within the scope of the present application, the above-mentioned technical features of the present application and the technical features specifically described below (such as embodiments) can be combined with each other to form new or preferred technical solutions. Due to space limitation, they will not be described one by one herein.

All publications, patents and patent applications mentioned herein are incorporated by reference to the same extent as if each individual publication, patent or patent application was specifically and individually indicated to be incorporated by reference. If there is a conflict between the terminology herein and the terminology of an incorporated reference, the terminology herein shall control.

After in-depth research, the inventor of the present application unexpectedly found that using a given signal peptide to construct a dimeric chimeric protein comprising a transmembrane domain can significantly improve the expression of the dimeric protein on cell surface. Signal peptides are usually used to improve protein expression and secretion, but the inventor of the present application unexpectedly discovered that the use of signal peptides in the introduced recombinant TCR can improve the expression of recombinant TCR with transmembrane domains on the cell surface and activate the TCR signaling pathway in response to target antigens, thereby solving the problem in the art that due to the low expression of exogenous TCRs on cell surface, the target antigen cannot effectively activate the TCR signaling pathway to achieve therapeutic effects.

### BRIEF DESCRIPTION OF THE DRAWINGS

The novel and inventive features of the disclosure are set forth particularly in the appended claims. A better understanding of the features and advantages of the disclosure will be obtained by reference to the following detailed description and accompanying drawings, which set forth illustrative embodiments in which the principles of the disclosure may be used.

FIG. 1. Positive rate detection of GPC3-TCR constructed with different signal peptides in Jurkat and J.RT-T3.5 cells.

FIG. 2. Positive rate detection of the recombinant TCR in GPC3-TCRT cells constructed with different signal peptides.

FIG. 3. GPC3-TCRT cells constructed with different signal peptides significantly kill target cells in vitro.

FIG. 4. Positive rate of the recombinant TCR is significantly increased in GPC3-TCRT cells with endogenous TCR knockout and constructed with different signal peptides.

FIG. 5. GPC3-TCRT cells with endogenous TCR knockout and constructed with different signal peptides significantly kill target cells in vitro.

FIG. 6. The inhibitory rate of GPC3-TCRT cells with endogenous TCR knockout on subcutaneous large-load liver transplanted tumors in mouse is significantly higher than that of GPC3-TCRT cells without endogenous TCR knockout.

FIG. 7. Under lymphodepletion pretreatment conditions, GPC3-TCRT cells with endogenous TCR knockout significantly inhibit subcutaneous liver transplanted tumors in mice.

FIG. 8. Under lymphodepletion pretreatment conditions, the inhibition rate of GPC3-TCRT cells with endogenous TCR knockout on subcutaneous large-load liver transplanted tumors in mouse is significantly higher than that of GPC3-CART cells.

FIG. 9A and FIG. 9B. GPC3-TCRT cells with endogenous TCR knockout and recombinant TCR comprising modified TCR constant regions significantly kill target cells in vitro and can secrete higher levels of cytokines IL-2, TNFα, and IFNγ.

FIG. 10A and FIG. 10B. Under lymphodepletion pretreatment conditions, the inhibitory rate of GPC3-TCRT cells with endogenous TCR knockout and recombinant TCR comprising modified TCR constant regions on subcutaneous liver transplanted tumors in mouse is significantly higher than that of GPC3-CART and GPC3-STAR-T cells.

FIG. 11A, FIG. 11B and FIG. 11C. GPC3-TCRT-IL12 cells with endogenous TCR knockout and recombinant TCR comprising modified TCR constant regions significantly kill target cells in vitro and can secrete higher levels of the cytokines IL-2, TNFα, and IFNγ.

FIG. 12A and FIG. 12B. GPRC5D-TCRT and BCMA-TCRT cells significantly kill target cells in vitro and can secrete higher levels of cytokines IL-2, TNFα, and IFNγ.

FIG. 13A and FIG. 13B. In the presence of NK cells, the inhibitory rate of GPRC5D-TCRT cells with endogenous TCR knockout on subcutaneous large-load myeloma transplanted in NPC mice is higher than that of GPRC5D-CART cells with endogenous TCR and B2M knockout.

FIG. 14A and FIG. 14B. NKG2A-TCRT cells with endogenous TCR and B2M knockout significantly kill NK (FIG. 14A); NKG2A-TCRT cells co-incubated with NK cells secrete higher levels of cytokines IL-2, TNFα, and IFNγ (FIG. 14B).

FIG. 15. Schematic diagram of IgGs-GPC3-TCR(lvivl)-NFAT-IL12 vector.

### DETAILED DESCRIPTION

The following description and examples provide a detailed explanation of the embodiments of the disclosure. It should be understood that this disclosure is not limited to the specific embodiments described herein. Those skilled in the art will recognize that there are many changes and modifications to this disclosure that are included within its scope. Unless otherwise stated, any embodiment may be combined with any other embodiment.

As used herein, certain inventive embodiments herein contemplate numerical ranges unless otherwise stated. Each aspect of the application may be presented in a range format. It should be understood that the description in range format is for convenience and brevity only and should not be construed as an inflexible limitation on the scope of the application. Therefore, descriptions of ranges should be considered to have specifically disclosed all possible subranges as well as the individual values within that range as if expressly written. For example, a description of a range such as 1-6 should be considered to have specifically disclosed subranges such as 1-3, 1-4, 1-5, 2-4, 2-6, 3-6, etc., and single numbers within the range, for example, 1, 2, 3, 4, 5, and 6. This applies regardless of the breadth of the scope. When a range exists, the range comprises the endpoints of the range.

The present application relates to an antibody recombinant TCR carrying an optimally selected signal peptide, and uses of engineered cells comprising the recombinant TCR. For example, tumor antigens (such as GPC3, GPRC5D, BCMA) or NK cell markers (such as NKG2A) are used as targets, and antibodies (such as antibody heavy chain variable region, or antibody light chain variable region) binding to different antigens and carrying different signal peptides are connected in series with the T cell receptor constant region (such as TRAC/TRBC, or TRGC/TRDC), and transferred into T cells to construct antibody-recombinant TCRT cells. In specific embodiments, the present application constructs Antibody-TCR-Chimeric T (ATCT) cells which express an Antibody-TCR-Chimeric (ATC) that responds to target antigen stimulation with high efficiency.

### 1. Definition

The term “activating immune effector cells” refers to changes in intracellular protein expression caused by signaling pathways, leading to initiation of an immune response. For example, CD3 molecules respond to ligand binding and aggregation of immunoreceptor tyrosine-based activation motifs (ITAMs), resulting in a signaling cascade. In one embodiment, the immune synapse formed by the binding of endogenous TCR or recombinant TCR with antigen comprises aggregation of many molecules near the binding receptor (e.g., CD4 or CD8, CD3γ/CDδ/CDε/CDζ, etc.). This aggregation of membrane-bound signaling molecules phosphorylates the ITAM motif comprised in the CD3 molecule. This phosphorylation initiates T cell activation pathways that ultimately activate transcription factors such as NF-κB and AP-1. These transcription factors induce the overall gene expression of T cells, including upregulating IL-2 production, promoting T cell proliferation, and thereby initiating T cell-mediated immune responses. “T cell activation” refers to the state of T cells that are stimulated to induce detectable cell proliferation, cytokine production, and/or detectable effector function. Using CD3/CD28 magnetic beads, either in vitro antigen stimulation or in vivo antigen stimulation will affect the degree and duration of T cell activation. In one embodiment, the engineered cells are activated either after co-incubation with cells comprising a specific target antigen or after being infected by a virus.

The term “to stimulate immune effector cells” refers to causing a strong and sustained immune response in immune effector cells through signaling pathways. In one embodiment, this occurs upon activation of immune effector cells (e.g., T cells) or through simultaneous mediation by receptors including, but not limited to, CD28, CD137 (4-1BB), OX40, CD40, and ICOS.

The term “antigen-binding domain” refers to molecules that specifically bind to antigenic determinants, including immunoglobulin molecules and the immune active portion of immune molecules, i.e., molecules that comprise an antigen-binding site that specifically binds to an antigen (“immune response”). The term “antibody” comprises not only whole antibody molecules, but also fragments of antibody molecules that retain antigen-binding ability. The term “antibody” is used interchangeably with the terms “immunoglobulin” and “antigen-binding domain” in the present application. Antibodies, including but not limited to monoclonal antibodies, polyclonal antibodies, natural antibodies, bispecific antibodies, chimeric antibodies,

Fv, Fab, Fab′, Fab′-SH, F(ab′) 2, linear antibodies, single chain antibody molecules (e.g., scFv), and single domain antibodies. In one embodiment, the antibody comprises at least two heavy (H) chains and two light (L) chains linked by disulfide bonds. Each heavy chain consists of a heavy chain variable region (VH) and a heavy chain constant region (CH). The heavy chain constant region consists of three domains: CHI, CH2, and CH3. Each light chain consists of a light chain variable region (VL) and a light chain constant region (CL). The light chain constant region consists of one domain. VH and VL can be further subdivided into hypervariable regions called complementarity-determining regions (CDRs), interspersed with more conservative regions called framework regions (FRs). Each VH and VL consists of three CDRs and four FRs, arranged in the following order from amino terminus to carboxyl terminus: FRI, CDR1, FR2, CDR2, FR3, CDR3, and FR4. The variable regions of the heavy and light chains comprise binding domains that interact with antigens. The constant regions of antibodies mediate binding of immunoglobulins with host tissues or factors (including various cells of the immune system (e.g., immune effector cells) and the first component (Clq) of the classical complement system). An antigen-binding domain “specifically binds” or is “immunoreactive” to an antigen if it binds the antigen with greater affinity than it binds to other reference antigens (including polypeptides or other substances).

The term “chimeric antigen receptor (CAR)” refers to a molecule that comprises an extracellular antigen-binding domain fused to an intracellular signaling domain capable of activating or stimulating immune effector cells and a transmembrane domain. In one embodiment, the extracellular antigen-binding domain of the CAR comprises scFV. scFV comprises an antibody heavy chain variable region and a light chain variable region. In one embodiment, the CAR comprises a polypeptide formed by sequentially connecting scFV, a transmembrane domain and an intracellular signaling domain.

The term “nucleic acid” or “polynucleotide” refers to a single-stranded or double-stranded deoxyribonucleic acid (DNA) or ribonucleic acid (RNA) and polymers thereof, including any nucleic acid molecule encoding a polypeptide of interest or a fragment thereof. The nucleic acid molecule only needs to maintain substantial identity to the endogenous nucleic acid sequence, and does not need to have 100% homology or identity to the endogenous nucleic acid sequence. A polynucleotide having “substantial identity” to the endogenous sequence can generally hybridize with at least one strand of a double-stranded nucleic acid molecule. “Hybridization” refers to the pairing of double-stranded molecules formed between complementary polynucleotide sequences, or portions thereof, under various stringent conditions. The term “homology” or “identity” refers to the identity of subunit sequences between two polymer molecules, for example, between two nucleic acid molecules, such as two DNA molecules or two RNA molecules, or between two polypeptide molecules. The term “substantial identity” or “substantial homology” refers to a polypeptide or nucleic acid molecule that exhibits at least about 50% homology or identity to a reference amino acid sequence or nucleic acid sequence. In one embodiment, such a sequence has at least about 60%, 65%, 70%, 75%, 80%, 85%, 90%, 95%, 99%, or 100% homology or identity with the amino acid or nucleic acid sequence used for comparison. Sequence identity can be measured by using sequence analysis software (e.g., BLAST, BESTFIT, GAP or PILEUP/PRETTYBOX programs). Such software matches identical or similar sequences by assigning degrees of homology to various substitutions, deletions and/or other modifications. Conservative substitutions typically comprise substitutions in below groups: glycine, alanine; valine, isoleucine, leucine; aspartic acid, glutamic acid, asparagine, glutamine; serine, threonine, lysine, arginine; and phenylalanine, tyrosine. In an exemplary method of determining the degree of identity, BLAST program may be used, wherein a probability score between e-3 and e-100 is indicative of closely related sequences.

The term “disease” refers to any disorder that damages or interferes with the normal function of a cell, tissue or organ, such as a tumor (cancer) or pathogen infection. Refractory cancers include, but are not limited to, cancers that are insensitive to radiotherapy, relapsed after radiotherapy, insensitive to chemotherapy, relapsed after chemotherapy, insensitive to CAR-T therapy, or relapsed after CAR-T treatment.

The terms “therapeutically effective amount”, “therapeutically effective”, “effective amount” or “in an effective amount” are used interchangeably herein and refer to the amount of a compound, preparation, substance, composition or pharmaceutical composition that is effective to achieve a specific biological result as described herein, such as but not limited to an amount or dose sufficient to promote T cell response. An effective amount of immune cells refers to, but is not limited to: the number of immune cells which can increase, enhance or prolong the anti-tumor activity; increase in the number of anti-tumor immune effector cells or activated immune effector cells; the number of immune effector cells which promote IFN-γ secretion, tumor regression, tumor shrinkage and tumor necrosis.

The term “endogenous” means that nucleic acid molecules or polypeptides etc. come from the organism itself.

The term “exogenous” means that a nucleic acid molecule or polypeptide is not endogenously present in the cell, or is not expressed at a level sufficient to achieve the function obtained by overexpressed; encompasses any recombinant nucleic acid molecule or polypeptide expressed in the cell, such as an exogenous, heterologous and overexpressed nucleic acid molecules and polypeptides.

The term “recognize” refers to selective binding to a target antigen. Engineered cells that recognize tumors can express receptors (such as recombinant TCRs) that bind to tumor antigens.

The term “specifically binds” refers to an antibody or ligand that recognizes and binds to a binding partner (e.g., tumor antigen) protein present in a sample, but the antibody or ligand does not substantially recognize or bind to other molecules in the sample.

The terms “individual” and “subject” are interchangeable and comprise humans or animals from other species including, but not limited to, humans, mice, rats, hamsters and guinea pigs, rabbits, dogs, cats, sheep, pigs, goats, cows, horses, apes, and monkeys.

The term “T cell (antigen) receptor (TCR)”, also known as “TCR subunit”, or “TCR unit”, is a characteristic marker on the surface of all T cells, which is non-covalently linked to CD3 to form TCR-CD3 complex. TCR is responsible for recognizing antigens bound to major histocompatibility complex molecules. TCR is a heterodimer consisting of two different peptide chains, the two peptide chains are a and B chains, or y and & chains; each peptide chain comprises a variable region and a constant region (comprising extracellular constant region, transmembrane region and cytoplasmic region); its characteristic is that the cytoplasmic region is very short. TCR molecules belong to the immunoglobulin superfamily, and their antigen specificity exists in the V region; the V region (Va, VB) each has three hypervariable regions, CDR1, CDR2, and CDR3. Among them, CDR3 has the largest variation, which directly determines the antigen binding specificity of TCR. When the TCR recognizes the MHC-antigen peptide complex, CDR1 and CDR2 recognize and bind to the side wall of the antigen-binding groove of the MHC molecule, while CDR3 directly binds to the antigen peptide. TCR is divided into two categories: TCR1 and TCR2; wherein TCR1 consists of γ and δ chains, while TCR2 consists of a and B chains. In peripheral blood, about 90%-95% of T cells express TCR2; and any T cell only expresses TCR2 or TCR1. The recognition ability of these natural TCR receptors is often weak and therefore cannot form an effective attack on target cells. In this case, the “affinity” of the natural TCR for the corresponding target antigen can be improved through partial genetic modification, that is, a high-affinity TCR, such as the recombinant TCR provided in the present application.

As used herein, “amino acid numbering refers to SEQ ID NO:x” (SEQ ID NO:x is a specific sequence listed herein) means that the position number of the specific amino acid described is the position number of the corresponding amino acid in SEQ ID NO:x. The correspondence of amino acids in different sequences can be determined according to sequence comparison methods known in the art. For example, the correspondence of amino acids can be determined through EMBL-EBI online alignment tool (https://www.ebi.ac.uk/Tools/psa/), wherein two sequences can be aligned using the Needleman-Wunsch algorithm and using default parameters. For example, if the amino acid at position 46 of a polypeptide from its N-terminus is aligned with the amino acid at position 47 of SEQ ID NO:x in a sequence alignment, the amino acid in the polypeptide may also be described herein as “alanine at position 48 of the polypeptide, the amino acid position refers to SEQ ID NO:x”.

The term “wild-type gene” refers to the allele that is the majority in nature and is often used as a standard control gene in biological experiments. The corresponding concept is mutant gene.

Wild-type TRAC nucleic acid molecule refers to the nucleic acid molecule encoding a natural TRAC polypeptide and having the nucleotide sequence shown in NCBI GenBank Gene ID: 28755, NG_001332.3, 925603 to 930229 (TRAC, SEQ ID NO: 19). Wild-type TRBC nucleic acid molecule refers to the nucleic acid molecule encoding a natural TRBC polypeptide and having the nucleotide sequence shown in NCBI GenBank Gene ID: 28639, NC_000007.14, 142791694 to 142793141 (TRBC1, SEQ ID NO: 21), or NCBI GenBank Gene ID: 28638, NG_001333.2, 655095 to 656583 (TRBC2). Wild-type TRGC nucleic acid molecule refers to the nucleic acid molecule encoding a natural TRGC polypeptide and having the nucleotide sequence shown in NCBI GenBank Gene ID: 6966, NG_001336.2, 108270 to 113860 (TRGC1, SEQ ID NO: 23), or NCBI GenBank Gene ID: 6967, NG_001336.2, 124376 to 133924 (TRGC2). Wild-type TRDC nucleic acid molecule refers to the nucleic acid molecule encoding a natural TRDC polypeptide and having the nucleotide sequence shown in NCBI GenBank Gene ID: 28526, NG_001332.3, 841011 to 844674 (TRDC, SEQ ID NO: 24).

The term “cysteine substitution” or “hydrophobic amino acid substitution” refers to the substitution of the original amino acid in the mentioned amino acid sequence (polypeptide or protein) with a cysteine or hydrophobic amino acid. Wherein the substitution of hydrophobic amino acid can be the substitution of hydrophilic amino acids with hydrophobic amino acids, or the substitution of low hydrophobicity amino acids with high hydrophobicity amino acids.

The term “isolated” means changed or removed from the natural state. For example, a nucleic acid or peptide naturally present in a living animal is not “isolated”, but the same nucleic acid or peptide that is partially or completely separated from a substance co-existing in its natural state is “isolated.” The isolated nucleic acid or protein may exist in a substantially purified form or may exist in a non-natural environment such as a host cell.

The terms “peptide,” “polypeptide,” and “protein” are used interchangeably and refer to compounds consisting of amino acid residues covalently linked by peptide bonds.

The term “synonymous mutation” means that the mutation of a certain base pair in a DNA fragment does not change the encoded amino acid because the codon at that position is a synonymous codon before and after the mutation. In a nucleic acid sequence, three consecutive nucleotide residues constitute a codon. Among the 64 codons, except for the 3 stop codons (TAA, TAG, TGA), the remaining 61 codons represent 20 amino acids. Except for methionine and tryptophan, which each have one codon, the other 18 amino acids have two or more codons. Different codons corresponding to the same amino acid are called synonymous codons. For example, the synonymous codons CTA and CTG both code for leucine. If the A in CTA is mutated to G, the mutation is a synonymous mutation.

The term “signal peptide (SP)”, or leader sequence, is a short peptide chain (about 5-30 amino acids in length) that guides transfer of newly synthesized proteins or polypeptides to the secretory pathway. SP is a short peptide located at the N-terminus (amino terminus) of a protein, which carries information about protein secretion and usually guides protein localization. The SP used herein preferably promotes secretion of the protein from the cell in which it is produced. After secretion from the cell, SP is usually cleaved from the rest of the protein (often called the mature protein).

The term “engineering” refers to an integrated science and technology using the principles and methods of cell biology and molecular biology to change the genetic material in cells or to obtain cell products according to people's wishes at the overall cell level, organelle level, and molecular level through some engineering means.

The term “transplant immune rejection” refers to the immune response of a host to an allogeneic tissue, organ, or cell transplantation, in which the exogenous transplant is recognized by the host's immune system as an “alien component” and was attacked, destroyed, and cleared by the host's immune system. The present application provides a cell that resists transplant immune rejection and a method for resisting the rejection.

The term “operably linked” refers to linking nucleic acid sequences in a manner or orientation that produces a nucleic acid molecule capable of directing the transcription of a given gene and/or the synthesis of a desired protein molecule. Two coding DNA sequences are said to be “operably linked” if the linkage results in contiguous translatable sequences without altering or interrupting the triplet reading frame. A coding sequence is operably linked to a gene expression element if the linkage results in appropriate function of the gene expression element, thereby causing expression of the DNA coding sequence.

The term “linker” comprises sequences encoding a self-cleaving peptide (e.g., 2A sequence) or a protease recognition site (e.g., furin). As used herein, “self-cleaving peptide” refers to an oligopeptide that allows multiple proteins to be encoded as a polyprotein, which dissociates into component proteins after translation. A variety of self-cleaving peptides are known to those skilled in the art, including, but not limited to, those found in members of the picornaviridae family, such as foot-and-mouth disease virus (FMDV), equine rhinitis A virus (ERAVO), thosea asigna virus (TaV) and Porcine Teschovirus-1 (PTV-1), and cardiac viruses such as theilovirus and encephalomyocarditis virus. The 2A peptides derived from FMDV, ERAV, PTV-1, and TaV are referred to herein as “F2A”, “E2A”, “P2A” and “T2A”, respectively. Those skilled in the art will be able to select self-cleaving peptides suitable for use in the present application. Exemplarily, F2A comprises the sequence of SEQ ID NO: 47, P2A comprises the sequence of SEQ ID NO: 46, and T2A comprises the sequence of SEQ ID NO: 48.

The term “nuclear factor of activated T cells (NFAT)” play an important role in transcriptional regulation of cytokine genes and cell surface receptors (for example, IL2, IL4, IL5, IL13, TNFα, IFNγ, GMCSF, CD40L, and CTLA-4, etc.). The NFAT proteins discovered so far can be divided into five types: NFAT1, NFAT2, NFAT3, NFAT4 and NFAT5. Among them, the activation of NFATc1-4 depends on the intracellular calcium signaling pathway.

The term “promoter” is defined as a DNA sequence recognized by the cell's synthetic machinery or introduced synthetic machinery required to initiate specific transcription of a polynucleotide sequence. A promoter is a DNA sequence that RNA polymerase recognizes, binds to, and starts transcription. It comprises conserved sequences required for specific binding of RNA polymerase and initiation of transcription.

The term “GPC3” refers to glypican 3, which is highly expressed specifically on liver cancer cells. The core protein of GPC3 is anchored to the cell membrane surface through glycosylphosphatidylinositol (GPI). The core protein of GPC3 can be cleaved into an N-terminal soluble protein (sGPC3) of approximately 40 KDa and a C-terminal membrane protein of 30 KDa. As used herein, “GPC3” refers to any variant, derivative or isoform of the GPC3 gene or protein encoded thereof. The PGC3 polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 2719, and/or may optionally comprise at most one or at most two or at most three conservative amino acid substitutions.

The term “GPRC5D” refers to G protein-coupled receptor class C group 5-member D. GPRC5D is specifically expressed on malignant bone marrow plasma cells. “GPRC5D” refers to any variant, derivative or isoform of the GPRC5D gene or encoded protein thereof. The GPRC5D polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 55507, and/or may optionally comprise at most one or at most two or at most three conservative amino acid substitutions.

The term “BCMA” refers to B-cell maturation antigen, which belongs to the TNF receptor superfamily. BCMA activates B cell proliferation and survival after binding to its ligand. BCMA is specifically highly expressed in plasma cells and multiple myeloma cells, but is not expressed in hematopoietic stem cells and other normal tissue cells. “BCMA” refers to any variant, derivative or isoform of the BCMA gene or protein encoded thereof. The BCMA polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 608, and/or may optionally comprise at most one or at most two or at most three conservative amino acid substitutions.

The term “NKG2A” refers to the NKG2A polypeptide, which is a member of the NKG2 transcript group. The heterodimeric inhibitory receptor CD94/NKG2A formed by NKG2A and CD94 is expressed on the surface of subsets of NK cells, αβT cells, γδT cells and NKT cells. As used herein, “NKG2A” refers to any variant, derivative or isoform of the NKG2A gene or protein encoded thereof. The NKG2A polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 3821, and/or may optionally comprise at most one or at most two or at most three conservative amino acid substitutions.

The term “interleukin 12 (IL12)” is a T cell stimulating factor. IL12 is a heterodimer consisting of the gene expression products of IL-12A (NCBI GenBank Gene ID: 3592) and IL-12B (NCBI GenBank Gene ID: 3593). The IL-12A polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 3592, and/or may optionally comprise at most one or at most two or at most three conservative amino acid substitutions; The IL-12B polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 3593, and/or may optionally comprise at most one or at most two or at most three conservative amino acid substitutions. In a specific example, IL12 has an amino acid sequence encoded by SEQ ID NO: 35 or 36.

### 2. Chimeric Polypeptides

Chimeric polypeptides in the present application refer to dimer molecules formed by connecting DNA fragments or corresponding cDNA of proteins or peptide fragments from different sources. The chimeric polypeptide of the present application comprises an A chain and a B chain, the A chain comprises a first antigen-binding domain and a first constant region, the B chain comprises a second antigen-binding domain and a second constant region; and the A chain and The B chain forms a dimer. In one embodiment, the upstream of the polynucleotide of the A or B chain or the amino terminus (N terminus) of the polypeptide is operably linked to a signal peptide (SP). In one embodiment, the upstream of the polynucleotide of the first antigen-binding domain or the second antigen-binding domain or the amino terminus (N-terminus) of the polypeptide is operably linked to a SP. In one embodiment, the first constant region and/or the second constant region comprise a transmembrane domain. In one embodiment, the first constant region and/or the second constant region comprise an intracellular domain. In one embodiment, the first constant region and/or the second constant region comprises a transmembrane domain and an intracellular domain.

In one embodiment, the chimeric polypeptides of the present application include, but are not limited to, recombinant TCR receptors. In one embodiment, the first constant region of the A chain of the recombinant TCR is a natural or modified T cell receptor a chain constant region (TRAC), and the second constant region of the B chain is a natural or modified T cell receptor β chain constant region (TRBC, such as TRBC1 or TRBC2). In one embodiment, the first constant region of the A chain of the recombinant TCR is a natural or modified T cell receptor y chain constant region (TRGC, such as TRGC1 or TRGC2), and the second constant region of the B chain is a natural or modified T cell receptor δ chain constant region (TRDC). In one embodiment, the chimeric polypeptide of the present application is also called antibody-TCR-chimeric (ATC).

### 2.1 Constant Region of the Recombinant TCR

In one embodiment, the first constant region of the recombinant TCR is a natural TRAC polypeptide, but the nucleic acid sequence is different from that of the wild-type TRAC, and/or the second constant region of the recombinant TCR is a natural TRBC polypeptide, but the nucleic acid sequence is different from that of the wild-type TRBC. In one embodiment, the first constant region of the recombinant TCR is a natural TRGC polypeptide, but the nucleic acid sequence is different from that of the wild-type TRGC, and/or the second constant region of the recombinant TCR is a natural TRDC polypeptide, but the nucleic acid sequence is different from that of the wild-type TRDC.

In one embodiment, the nucleic acid molecule of the first constant region and/or the second constant region of the recombinant TCR comprises a nucleic acid molecule that is no longer the target sequence targeted by gene knockout technology and/or gene silencing technology after nucleotide mutation. In one embodiment, the nucleic acid molecule of the first constant region and/or the second constant region of the recombinant TCR comprises a nucleic acid molecule that is no longer the target sequence targeted by gene knockout technology and/or gene silencing technology after synonymous mutation of the nucleotide(s).

In one embodiment, synonymous mutations are performed on the wild-type TRAC and/or TRBC nucleic acid fragments comprised in the A chain and/or B chain of the recombinant TCR. In one embodiment, synonymous mutations are performed on the wild-type TRGC and/or TRDC nucleic acid fragments comprised in the A chain and/or B chain of the recombinant TCR.

In one embodiment, hydrophobic amino acid substitutions are performed on the transmembrane region of the first constant region and/or the second constant region of the recombinant TCR to increase the stability of the recombinant TCR molecule.

In one embodiment, the first constant region of the recombinant TCR comprises hydrophobic amino acid substitutions within the transmembrane region relative to natural TRAC. In one embodiment, the first constant region of the recombinant TCR comprises hydrophobic amino acid substitutions at positions 115, 118, and/or 119 relative to natural TRAC. In one embodiment, the first constant region of the recombinant TCR comprises hydrophobic amino acid substitutions at positions 115, 118, and 119 relative to natural TRAC. In one embodiment, relative to natural TRAC, the first constant region of the recombinant TCR has serine at position 115 replaced by leucine, glycine at position 118 replaced by valine, and/or proline at position 119 replaced by leucine. In one embodiment, relative to natural TRAC, the first constant region of the recombinant TCR has serine at position 115 replaced by leucine, glycine at position 118 replaced by valine, and proline at position 119 replaced by leucine. The above amino acid numbering refers to SEQ ID NO: 19. In one embodiment, the first constant region of the recombinant TCR is as shown in SEQ ID NO: 26, 28, 29 or 31.

In one embodiment, cysteine point mutations are performed on the first constant region and/or the second constant region of the recombinant TCR to introduce intermolecular disulfide bonds, enhance the mutual pairing between the A and B chains of the recombinant TCR molecule, and reduce mismatch with endogenous TCR.

In one embodiment, relative to natural TRAC, the threonine T at position 47 of the first constant region of the recombinant TCR is mutated to cysteine C, and the amino acid numbering refers to SEQ ID NO: 19; relative to natural TRBC, the serine S at position 57 of the second constant region of the recombinant TCR is mutated to cysteine C, and the amino acid numbering refers to SEQ ID NO: 21. In one embodiment, the first constant region of the recombinant TCR is as shown in SEQ ID NO: 27, 28, 30 or 31; and/or the second constant region of the recombinant TCR is as shown in SEQ ID NO: 33 or 34.

In one embodiment, hydrophobic amino acid substitutions and cysteine point mutations are performed in the transmembrane region of the first constant region and/or the second constant region of the recombinant TCR to increase the stability of the recombinant TCR molecule and reduce the mismatch between the recombinant TCR and endogenous TCR. In one embodiment, the first constant region of the recombinant TCR is as shown in SEQ ID NO: 30 or 31; and/or the second constant region of the recombinant TCR is as shown in SEQ ID NO: 34.

In one embodiment, the first constant region comprised in the recombinant TCR polypeptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 19, and/or may optionally comprise at most one or at most two or at most three conservative amino acid substitutions.

In one embodiment, the first constant region comprised in the recombinant TCR polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 28755, NG_001332.3, 925603 to 930229, and/or may optionally comprise at most one or at most two or at most three conservative amino acid substitutions.

In one embodiment, the second constant region comprised in the recombinant TCR polypeptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 21, and/or may optionally comprise at most one or at most two or at most three conservative amino acid substitutions.

In one embodiment, the second constant region comprised in the recombinant TCR polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI GenBank Gene ID: 28639, NC_000007.14, 142791694 to 142793141 (TRBC1, SEQ ID NO: 21), or NCBI GenBank Gene ID: 28638, NG_001333.2, 655095 to 656583 (TRBC2), and/or may optionally comprise at most one or at most two or at most three conservative amino acid substitutions.

In one embodiment, the first constant region comprised in the recombinant TCR polypeptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 23, and/or may optionally comprise at most one or at most two or at most three conservative amino acid substitutions.

In one embodiment, the first constant region comprised in the recombinant TCR polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI Genbank ID: 6966, NG_001336.2, 108270 to 113860 (TRGC1), or NCBI Genbank ID: 6967, NG_001336.2, 124376 to 133924, and/or may optionally comprise at most one or at most two or at most three conservative amino acid substitutions.

In one embodiment, the second constant region comprised in the recombinant TCR polypeptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence of SEQ ID NO: 24, and/or may optionally comprise at most one or at most two or at most three conservative amino acid substitutions.

In one embodiment, the second constant region comprised in the recombinant TCR polypeptide has an amino acid sequence or a fragment thereof having at least about 80%, at least about 85%, at least about 90%, at least about 95%, at least about 96%, at least about 97%, at least about 98%, at least about 99%, or at least about 100% homology or identity to an amino acid sequence encoded by the transcript expressed by the gene of NCBI Genbank ID: 28526, NG 001332.3, 841011-844674, and/or may optionally comprise at most one or at most two or at most three conservative amino acid substitutions.

In one embodiment, the A chain of the recombinant TCR comprises a first antigen-binding domain directly or indirectly connected with the first constant region; and the B chain of the recombinant TCR comprises a second antigen-binding domain directly or indirectly connected with the second constant region. In one embodiment, the antigen-binding domain is connected with the hinge/spacer region of the first/second constant region. The hinge/spacer region may be a hinge region from IgG1, or a CH2CH3 region and a portion of CD3 of an immunoglobulin, a portion of a CD28 polypeptide, a portion of a CD8 polypeptide, a variant having at least about 80%, at least about 85%, at least about 90% or at least about 95% homology or identity to any of the foregoing, or a synthetic spacer sequence.

In one embodiment, the recombinant TCR polypeptide comprises TRAC (SEQ ID NO: 19) and TRBC (SEQ ID NO: 21). In one embodiment, the recombinant TCR does not comprise the nucleotide sequence of SEQ ID NO: 20 and/or SEQ ID NO: 22. In one embodiment, the recombinant TCR polypeptide comprises TRAC (SEQ ID NO: 19) and TRBC (SEQ ID NO: 21), but the recombinant TCR does not comprise the nucleotide sequence of SEQ ID NO: 20 and/or SEQ ID NO: 22.

In one embodiment, the nucleic acid sequence of the recombinant TCR comprises: TRAC nucleic acid fragment 1 (SEQ ID NO: 25), TRAC nucleic acid fragment 2 (SEQ ID NO: 26), TRAC nucleic acid fragment 3 (SEQ ID NO: 27), TRAC nucleic acid Fragment 4 (SEQ ID NO: 28), TRAC nucleic acid fragment 5 (SEQ ID NO: 29), TRAC nucleic acid fragment 6 (SEQ ID NO: 30) or TRAC nucleic acid fragment 7 (SEQ ID NO: 31).

In one embodiment, the nucleic acid sequence of the recombinant TCR comprises: TRBC nucleic acid fragment 1 (SEQ ID NO: 32), TRBC nucleic acid fragment 2 (SEQ ID NO: 33) or TRBC nucleic acid fragment 3 (SEQ ID NO: 34).

In one embodiment, the first constant region comprises the sequence encoded by SEQ ID NO: 26 or 29, and the second constant region comprises the sequence encoded by SEQ ID NO: 32. In one embodiment, the first constant region comprises the sequence encoded by SEQ ID NO: 27, 28, 30 or 31, and the second constant region comprises the sequence encoded by SEQ ID NO: 33 or 34.

In one embodiment, the nucleic acid sequence of the recombinant TCR comprises TRAC nucleic acid fragment 1 (SEQ ID NO: 25), TRAC nucleic acid fragment 2 (SEQ ID NO: 26) or TRAC nucleic acid fragment 5 (SEQ ID NO: 29); and TRBC nucleic acid fragment 1 (SEQ ID NO: 32). In one embodiment, the nucleic acid sequence of the recombinant TCR comprises TRAC nucleic acid fragment 3 (SEQ ID NO: 27), TRAC nucleic acid fragment 4 (SEQ ID NO: 28), TRAC nucleic acid fragment 6 (SEQ ID NO: 30) or TRAC nucleic acid fragment 7 (SEQ ID NO: 31); and TRBC nucleic acid fragment 2 (SEQ ID NO: 33) or TRBC nucleic acid fragment 3 (SEQ ID NO: 34).

In one embodiment, recombinant TCR polypeptides comprise TRGC (SEQ ID NO: 23) and TRDC (SEQ ID NO: 24).

The extracellular domain of the recombinant TCR of the present application can be derived from natural sources or recombinant sources. In the case of natural source, the domain may be derived from any protein, but in particular membrane-bound or transmembrane proteins. In one aspect, the extracellular domain is capable of associating with a transmembrane domain. Extracellular domains that are particularly useful in the present application may comprise at least the following extracellular region: for example, the α, β or γ, δ chain of a T cell receptor, or CD3ε, CD3γ or CD3δ, or in alternative embodiments, comprising CD28, CD45, CD4, CD5, CD8, CD9, CD16, CD22, CD33, CD37, CD64, CD80, CD86, CD134, CD137, or CD154.

The transmembrane domain of the recombinant TCR of the present application can be derived from natural sources or recombinant sources. In the case of natural source, the domain may be derived from any membrane-bound or transmembrane protein. In one aspect, the transmembrane domain is capable of signaling to the intracellular domain whenever the recombinant TCR binds to the target antigen. Transmembrane domains that are particularly useful in the present application may comprise at least the following transmembrane regions: for example, the α, β or γ, δ chain of a T cell receptor, or CD3ε, CD3γ, CD3δ, CD28, CD45, CD4, CD5, CD8, CD9, CD16, CD22, CD33, CD37, CD64, CD80, CD86, CD134, CD137, or CD154. In some cases, the transmembrane domain can be connected with the extracellular region of the recombinant TCR (e.g., the antigen-binding domain of the recombinant TCR) via a hinge (e.g., a hinge from a human protein). For example, in one embodiment, the hinge may be the hinge of the α or β chain of the T cell receptor.

2.2 Extracellular Antigen-Binding Domain The first and second antigen-binding domains comprised in the recombinant [176] TCR of the present application bind to the same antigen or different antigens. In one embodiment, the antigen-binding domain binds to tumor antigens, pathogen antigens, and/or NK cell markers. In one embodiment, the antigen-binding domain comprises an antibody or a fragment thereof. In one embodiment, the antigen-binding domain comprises an antibody heavy chain variable region (VH) and/or a light chain variable region (VL); or comprises a cross-linked Fab; or comprises F(ab)₂. In one embodiment, the antigen-binding domain comprise antibody VH and VL respectively, forming a variable fragment (Fv).

In one embodiment, the antibody VH in the recombinant TCR is directly and/or indirectly connected with TRAC, and/or the antibody VL is directly and/or indirectly connected with TRBC. In one embodiment, the antibody VH in the recombinant TCR is directly and/or indirectly connected with TRBC, and/or the antibody VL is directly and/or indirectly connected with TRAC. In one embodiment, the antibody VH in the recombinant TCR is directly and/or indirectly connected with TRGC, and/or the antibody VL is directly and/or indirectly connected with TRDC. In one embodiment, the antibody VH in the recombinant TCR is directly and/or indirectly connected with TRDC, and/or the antibody VL is directly and/or indirectly connected with TRGC.

In one embodiment, the recombinant TCR comprises the heavy chain variable region encoded by SEQ ID NO: 38 and the light chain variable region encoded by SEQ ID NO: 39. In one embodiment, the recombinant TCR comprises the heavy chain variable region encoded by SEQ ID NO: 40 and the light chain variable region encoded by SEQ ID NO: 41. In one embodiment, the recombinant TCR comprises the heavy chain variable region encoded by SEQ ID NO: 42 and the light chain variable region encoded by SEQ ID NO: 43. In one embodiment, the recombinant TCR comprises the heavy chain variable region encoded by SEQ ID NO: 44 and the light chain variable region encoded by SEQ ID NO: 45.

### 2.3 Signal Peptide (SP)

The polynucleotide coding regions of the A chain and B chain of the recombinant TCR of the present application can be connected with the coding region encoding SP, and the SP directs the secretion of the A and B chains of the recombinant TCR of the present application. For example, if secretion of the fusion protein is required, the polynucleotide encoding the SP can be placed upstream of the polynucleotide encoding the A and B chains of the recombinant TCR. In one embodiment, the coding sequence of SP and the coding sequence of the A and B chains of the recombinant TCR are operably linked, so that the protein product produced is a functional protein product with the desired amino acid sequence. In one embodiment, the A and B chains of the recombinant TCR are directly connected with SP, or connected with SP through a linker.

In one embodiment, the SP is a natural signal peptide from a member of the wild-type immunoglobulin superfamily (IgSF). In one embodiment, SP is modified from the natural signal peptide of IgSF. In one embodiment, the natural SP of IgSF includes, but is not limited to: signal peptide of PD-L1, PD-L2, CD80, CD86, ICOS ligand, B7-H3, B7-H4, CD28, CTLA4, PD-1, ICOS, BTLA, CD4, CD8-α, CD8-β, LAG3, TIM-3, CEACAM1, TIGIT, PVR, PVRL2, CD226, CD2, CD160, CD200, CD200R, TCR, NKp30, or growth factors. In one embodiment, the growth factor SP includes, but is not limited to: signal peptide of macrophage colony stimulating factor (MCSF), granulocyte colony stimulating factor (GCSF), granulocyte macrophage colony stimulating factor (GMCSF), CD2 or ICAM.

In one embodiment, the SP of the A chain of the recombinant TCR comprising the TRAC polypeptide is the signal peptide of TRAV, i.e., TRAVs (SEQ ID NO: 1), and the SP of the B chain of the recombinant TCR comprising the TRBC polypeptide is the signal peptide of TRBV, i.e., TRBVs (SEQ ID NO. NO: 3). In one embodiment, the SP of the A chain of the recombinant TCR comprising the TRGC polypeptide is TRGVs, and the SP of the B chain of the recombinant TCR of the TRDC polypeptide is TRDVs. In one embodiment, both the SPs of the two chains are the signal peptide of GMCSF, i.e., GMCSFs (SEQ ID NO: 5). In one embodiment, both the SPs of the two chains are the signal peptide of GMCSFRa, i.e., GMCSFRas (SEQ ID NO: 7).

In one embodiment, SPs of the two chains are secretory signal peptides that promote secretion of the heavy and light chains of the antibody. In one embodiment, the SP is the heavy chain signal peptide of natural IgG, IgM, IgD, IgA or IgE. In one embodiment, SPs of the two chains are the signal peptide of the natural K or A chain. In one embodiment, the SP connected with the antibody VL is the signal peptide of a natural K light chain, such as IgGsL1 (SEQ ID NO: 9), IgGsL2 (SEQ ID NO: 11), or IgGsL3 (SEQ ID NO: 12). In one embodiment, the SP connected with the antibody VL is the signal peptide of a natural 2 light chain, such as IgGsL4 (SEQ ID NO: 13).

In one embodiment, the A chain of the recombinant TCR comprises: SP that promotes secretion of the antibody heavy chain, antibody VH, and natural or modified TRAC constant region; and/or, the B chain comprises: SP that promotes secretion of the antibody light chain, antibody VL, and natural or modified TRBC constant region.

In one embodiment, the A chain of the recombinant TCR comprises: SP that promotes the secretion of the antibody light chain, antibody VL, and the natural or modified TRAC constant region; and/or the B chain comprises: SP that promotes the secretion of the antibody heavy chain, antibody VH, and natural or modified TRBC constant region.

In one embodiment, the A chain of the recombinant TCR comprises: TRAVs (SEQ ID NO: 1), antibody VH, and natural or modified TRAC constant region; and/or the B chain comprises: TRBVs (SEQ ID NO: 3), antibody VL, and natural or modified TRBC constant region. In one embodiment, the A chain of the recombinant TCR comprises: TRAVs (SEQ ID NO: 1), antibody VL, and natural or modified TRAC constant region; and/or the B chain comprises: TRBVs (SEQ ID NO: 3), antibody VH, and natural or modified TRBC constant region.

In one embodiment, the A chain of the recombinant TCR comprises: TRGVs, antibody VH, and natural or modified TRGC constant regions; and/or the B chain comprises: TRDVs, antibody VL, and natural or modified TRDC constant regions. In one embodiment, the A chain of the recombinant TCR comprises: TRGVs, antibody VL, and natural or modified TRGC constant region; and/or the B chain comprises: TRDVs, antibody VH, and natural or modified TRDC constant regions.

In one embodiment, the SP of the A chain or B chain of the recombinant TCR comprising the antigen-binding domain of the antibody VH is selected from: IgGsH1 (SEQ ID NO: 14), IgGsH2 (SEQ ID NO: 16), IgGsH3 (SEQ ID NO: 17), or IgGsH4 (SEQ ID NO: 18). In one embodiment, the SP of the A chain or B chain of the recombinant TCR comprising the antigen-binding domain of the antibody VL is selected from: IgGsL1 (SEQ ID NO: 9), IgGsL2 (SEQ ID NO: 11), IgGsL3 (SEQ ID NO: 12), or IgGsL4 (SEQ ID NO: 13).

In one embodiment, the SP of the A chain or B chain of the recombinant TCR comprising the antigen-binding domain of the antibody VH is selected from: IgGsH1 (SEQ ID NO: 14), IgGsH2 (SEQ ID NO: 16) or IgGsH3 (SEQ ID NO: 17); and the SP of the A chain or B chain of the recombinant TCR comprising the antigen-binding domain of the antibody VL is IgGsL1 (SEQ ID NO: 9).

In one embodiment, the SP of the A chain or B chain of the recombinant TCR comprising the antigen-binding domain of the antibody VH is IgGsH4 (SEQ ID NO: 18); and the SP of the A chain or B chain of the recombinant TCR comprising the antigen-binding domain of the antibody VL is IgGsL3 (SEQ ID NO: 12) or IgGsL4 (SEQ ID NO: 13).

In one embodiment, the amino terminus of the antibody VH and/or VL in the recombinant TCR is directly or indirectly connected with GMCSFs (SEQ ID NO: 5).

In one embodiment, the amino terminus of the antibody VH in the recombinant

TCR is directly or indirectly connected with IgGsH1 (SEQ ID NO: 14), IgGsH2 (SEQ ID NO: 16), IgGsH3 (SEQ ID NO: 17) or IgGsH4 (SEQ ID NO: 18).

In one embodiment, the amino terminus of the antibody VL in the recombinant TCR is directly or indirectly connected with IgGsL1 (SEQ ID NO: 9), IgGsL2 (SEQ ID NO: 11), IgGsL3 (SEQ ID NO: 12) or IgGsL4 (SEQ ID NO: 13).

In one embodiment, the amino terminus of the antibody VH in the recombinant TCR is directly or indirectly connected with IgGsH1 (SEQ ID NO: 14), IgGsH2 (SEQ ID NO: 16), IgGsH3 (SEQ ID NO: 17) or IgGsH4 (SEQ ID NO: 18); and the amino terminus of the antibody VL in the recombinant TCR is directly or indirectly connected with IgGsL1 (SEQ ID NO: 9), IgGsL2 (SEQ ID NO: 11), IgGsL3 (SEQ ID NO: 12) or IgGsL4 (SEQ ID NO: 13).

In one embodiment, the amino terminus of the antibody VH in the recombinant TCR is directly or indirectly connected with IgGsH1 (SEQ ID NO: 14), IgGsH2 (SEQ ID NO: 16) or IgGsH3 (SEQ ID NO: 17); and the amino terminus of the antibody VL in the recombinant TCR is directly or indirectly connected with IgGsL1 (SEQ ID NO: 9).

In one embodiment, the amino terminus of the antibody VH in the recombinant TCR is directly or indirectly connected with IgGsH4 (SEQ ID NO: 18); and the amino terminus of the antibody VL in the recombinant TCR is directly or indirectly connected with IgGsL3 (SEQ ID NO: 12) or IgGsL4 (SEQ ID NO: 13).

In one embodiment, SP amino acid sequence may comprise at least 1, at least 2, at least 3, at least 4, or at least 5 mutations. In one embodiment, the recombinant TCR of the present application comprises SP bound to antigen-binding domain. In one embodiment, the SP comprises one or two or three or four mutations. Mutations comprise altering the nucleotide sequence of the natural signal peptide thereby changing the encoded amino acid (missense mutation), deleting an amino acid from the signal peptide sequence, or inserting a new amino acid into the natural signal peptide sequence.

The present application comprises recombinant DNA molecules encoding the recombinant TCR. Exemplarily, the recombinant TCR comprises an antibody fragment that binds to a tumor antigen, wherein the nucleic acid sequences encoding the antibody fragment, a signal peptide, the first and second constant regions are connected and are in the same open reading frame (Open Reading Frame).

In one embodiment, the recombinant DNA molecule of the recombinant TCR comprises: (1) antibody heavy chain signal peptide, antibody VH, wild-type TRAC or mutated nucleic acid fragment thereof, linker polypeptide, antibody light chain signal peptide, antibody VL, wild-type TRBC or mutated nucleic acid fragment thereof; (2) antibody heavy chain signal peptide, antibody VH, wild-type TRBC or mutated nucleic acid fragment thereof, linker polypeptide, antibody light chain signal peptide, antibody VL, wild-type TRAC or mutated nucleic acid fragment thereof; (3) antibody light chain signal peptide, antibody VL, wild-type TRBC or mutated nucleic acid fragment thereof, linker polypeptide; antibody heavy chain signal peptide, antibody VH, wild-type TRAC or mutated nucleic acid fragment thereof; or (4) antibody light chain signal peptide, antibody VL, wild-type TRAC or mutated nucleic acid fragment thereof, linker polypeptide; antibody heavy chain signal peptide, antibody VH, wild-type TRBC or mutated nucleic acid fragment thereof.

In one embodiment, the recombinant DNA molecule of the recombinant TCR comprises: (1) TRAVs, antibody VH, wild-type TRAC or mutated nucleic acid fragment thereof, linker polypeptide, TRBVs, antibody VL, wild-type TRBC or mutated nucleic acid fragment thereof; (2) TRBVs, antibody VH, wild-type TRBC or mutated nucleic acid fragment thereof, linker polypeptide, TRAVs, antibody VL, wild-type TRAC or mutated nucleic acid fragment thereof; (3) TRAVs, antibody VL, wild-type TRBC or mutated nucleic acid fragment thereof, linker polypeptide; TRBVs, antibody VH, wild-type TRAC or mutated nucleic acid fragment thereof; or (4) TRBVs, antibody VL, wild-type TRAC or mutated nucleic acid fragment thereof, linker polypeptide; TRAVs, antibody VH, wild-type TRBC or mutated nucleic acid fragment thereof.

In one embodiment, antibodies that recognize tumor antigens are provided. Exemplarily, the antibody that recognizes GPC3 comprises the nucleic acid sequences of VH (SEQ ID NO: 38), and VL (SEQ ID NO: 39) and the amino acid sequences encoded thereof. Exemplarily, the antibody that recognizes GPRC5D comprises the nucleic acid sequences of VH (SEQ ID NO: 40), and VL (SEQ ID NO: 41) and the amino acid sequences encoded thereof. Exemplarily, the antibody that recognizes BCMA comprises the nucleic acid sequences of VH (SEQ ID NO: 42), and VL (SEQ ID NO: 43) and the amino acid sequences encoded thereof. In one embodiment, provided are antibodies recognizing NK cells. Exemplarily, the antibody that recognizes NKG2A comprises the nucleic acid sequences of VH (SEQ ID NO: 44), and VL (SEQ ID NO: 45) and the amino acid sequences encoded thereof.

The present application contemplates modification of the entire recombinant TCR molecule, for example, modification of one or more amino acid sequences of each domain of the recombinant TCR molecule, so as to produce a functionally equivalent molecule. The recombinant TCR molecule can be modified to retain at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to the starting recombinant TCR molecule.

The sequence provided in the present application is not limited to the recombinant TCR with a specific amino acid sequence described in the present application, and the recombinant TCR with an amino acid sequence that has been modified and/or replaced with one or more amino acids, and/or deleted and/or added with one or more amino acids on the basis of the specific amino acid sequence, and has 60%, 65%, 70%, 75%, 80%, 85%, 90%, 95% or more identity to the specific amino acid sequence, and have the same function, is also within the protection scope of the present application.

One of ordinary skill in the art will appreciate that the antibodies or antibody fragments of the present application can be further modified so that they vary in amino acid sequences (e.g., relative to wild type) but not in the desired activity. For example, additional nucleotide substitutions can be made to the protein, resulting in amino acid substitutions at “non-essential” amino acid residues. For example, a non-essential amino acid residue in a molecule can be replaced by another amino acid residue from the same side chain family. In another embodiment, the amino acid fragments may be replaced by amino acid fragments that are structurally similar but differ in sequence and/or composition from the side chain family members, e.g., conservative substitutions may be made in which the amino acid residues are replaced by amino acid residues with similar side chains.

### 2.3. Antigen

In one embodiment, the recombinant TCR binds to a tumor antigen. Any tumor antigen may be used in the tumor-related examples described herein. Antigens are expressed as polypeptides or intact proteins or parts thereof. The tumor antigens of the present application comprise but are not limited to: thyroid stimulating hormone receptor (TSHR); CD171; CS-1; C-type lectin-like molecule-1; ganglioside GD3; Tn antigen; CD19; CD20; CD22; CD30; CD70; CD123; CD138; CD33; CD44; CD44v7/8; CD38; CD44v6; B7H3 (CD276), B7H6; KIT (CD117); interleukin 13 receptor subunit α (IL-13Rα); interleukin 11 receptor α (IL-11Rα); prostate stem cell antigen (PSCA); prostate specific membrane antigen (PSMA); carcinoembryonic antigen (CEA); NY-ESO-1; HIV-1 Gag; MART-1; gp100; tyrosinase; mesothelin; EpCAM; protease serine 21 (PRSS21); vascular endothelial growth factor receptor, vascular endothelial growth factor receptor 2 (VEGFR2); Lewis (Y) antigen; CD24; platelet derived growth factor receptor β (PDGFR-β); stage-specific embryonic antigen-4 (SSEA-4); cell surface-associated mucin 1 (MUC1), MUC6; epidermal growth factor receptor family and its mutants (EGFR, EGFR2, ERBB3, ERBB4, EGFRvIII); neural cell adhesion molecule (NCAM); carbonic anhydrase IX (CAIX); LMP2; ephrin A receptor 2 (EphA2); fucosyl GM1; sialyl Lewis adhesion molecule (sLe); ganglioside GM3 (aNeu5Ac (2-3) bDGalp (1-4) bDGlcp (1-1) Cer; TGS5; high molecular weight melanoma-associated antigen (HMWMAA); O-acetyl GD2 ganglioside (OAcGD2); folate receptor; tumor vascular endothelial marker 1 (TEM1/CD248); tumor vascular endothelial marker 7 related (TEM7R); Claudin 6, Claudin 18.2, Claudin 18.1; ASGPR1; CDH16; 5T4; 8H9; ανβ6 integrin; B cell maturation antigen (BCMA); CA9; kappa light chain; CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variant of tumor necrosis zone; G protein coupled receptor C class 5 group-member D (GPRC5D); chromosome X open reading frame 61 (CXORF61); CD97; CD179a; anaplastic lymphoma kinase (ALK); polysialic acid; placenta specific 1 (PLAC1); hexose part of globoH glycoceramide (GloboH); breast differentiation antigen (NY-BR-1); uroplakin 2 (UPK2); hepatitis A virus cell receptor 1 (HAVCR); adrenergic receptor β3 (ADRB3); pannexin 3 (PANX3); G protein-coupled receptor 20 (GPR20); lymphocyte antigen 6 complex locus K9 (LY6K); olfactory receptor 51E2 (OR51E2); TCRγ alternating reading frame protein (TARP); wilms tumor protein (WT1); ETS translocation variant gene 6 (ETV6-AML); sperm protein 17 (SPA17); X antigen family member 1A (XAGE1); angiopoietins bind to cell surface receptor 2 (Tie2); melanoma cancer testis antigen-1 (MAD-CT-1); melanoma cancer testis antigen-2 (MAD-CT-2); Fos related antigen 1; p53 mutant; human telomerase reverse transcriptase (hTERT); sarcoma translocation breakpoint; melanoma inhibitor of apoptosis (ML-IAP); ERG (transmembrane protease serine 2 (TMPRSS2) ETS fusion gene); N-acetylglucosaminyl transferase V (NA17); paired box protein 3 (PAX3); androgen receptor; cyclin B1; V-myc avian myelocytomatosis virus oncogene neuroblastoma derived homolog (MYCN); Ras homolog family member C (RhoC); cytochrome P450 1B1 (CYP1B1); CCCTC binding factor (zinc finger protein)-like (BORIS); squamous cell carcinoma antigen 3 recognized by T cells (SART3); paired box protein 5 (PAX5); proacrosin binding protein sp32 (OYTES1); lymphocyte-specific protein tyrosine kinase (LCK); A kinase anchoring protein 4 (AKAP-4); synovial sarcoma X breakpoint 2 (SSX2); CD79a; CD79b; CD72; leukocyte-associated immunoglobulin-like receptor 1 (LAIR1); Fc fragment of IgA receptor (FCAR); leukocyte immunoglobulin-like receptor subfamily member 2 (LILRA2); CD300 molecular-like family member f (CD300LF); C-type lectin domain family 12 member A (CLEC12A); bone marrow stromal cell antigen 2 (BST2); EGF-like module containing mucin-like hormone receptor 2 (EMR2); lymphocyte antigen 75 (LY75); glypican-3 (GPC3); Fc receptor-like 5 (FCRL5); immunoglobulin lambda-like polypeptide 1 (IGLL1).

In one embodiment, the recombinant TCR recognizes a pathogen antigen, e.g., for use in treating and/or preventing pathogen infection or other infectious disease, e.g., in an immunocompromised subject. Pathogen antigen includes, but is not limited to: antigen of viruses, bacteria, fungi, protozoa, or parasites; and viral antigen includes, but is not limited to: cytomegalovirus (CMV) antigen, Epstein-Barr virus (EBV) antigen, human immune defective virus (HIV) antigen or influenza virus antigen.

In one embodiment, the recombinant TCR recognizes NK cell markers, for example, for anti-transplant immune rejection, and particularly relates to a method for resisting anti-NK cell immune rejection. In one embodiment, TCRT cells targeting NK cells can be used to treat, prevent or improve autoimmune diseases or inflammatory diseases, especially inflammatory diseases related to autoimmune diseases, such as arthritis (e.g., rheumatoid arthritis, arthritis chronica progrediente and arthritis deformans) and rheumatic diseases, including inflammatory conditions and rheumatic diseases involved in bone loss and inflammatory pain, spondyloarthropathies (including ankylosing spondylitis), Reiter's syndrome, reactive arthritis, psoriatic arthritis, juvenile idiopathic arthritis and enteropathic arthritis, enthesitis, hypersensitivity (including airway hypersensitivity and skin hypersensitivity) and allergies. The engineered T cells provided by the present application are used for treating and preventing autoimmune hematological disorders (including, for example, hemolytic anemia, aplastic anemia, pure red blood cell anemia and idiopathic thrombocytopenia), systemic lupus erythematosus (SLE), lupus nephritis, inflammatory muscle disease (dermatomyositis), periodontitis, polychondritis, scleroderma, Wegener's granulomatosis, dermatomyositis, chronic active hepatitis, myasthenia gravis, psoriasis, Stevens-Johnson syndrome, idiopathic sprue, autoimmune inflammatory bowel disease (including, for example, ulcerative colitis, Crohn's disease, and irritable bowel syndrome), endocrine ophthalmopathy, Graves' disease, sarcoidosis, multiple sclerosis, systemic sclerosis, fibrotic diseases, primary biliary cirrhosis, juvenile diabetes mellitus (type I diabetes), uveitis, keratoconjunctivitis sicca and vernal keratoconjunctivitis, interstitial pulmonary fibrosis, periprosthetic osteolysis, glomerulonephritis (with and without nephrotic syndrome, including, for example, idiopathic nephrotic syndrome or minimal change nephropathy), multiple myeloma, other types of tumors, Inflammatory diseases of skin and cornea, myositis, loosening of bone implants, metabolic disorders (such as obesity, atherosclerosis and other cardiovascular diseases including dilated cardiomyopathy, myocarditis, type II diabetes and dyslipidemia) and autoimmune thyroid diseases (including Hashimoto's thyroiditis), primary vasculitis of small and medium vessels, large vessel vasculitis including giant cell arteritis, hidradenitis suppurativa, neuromyelitis optica, Sjogren's syndrome, Behcet's diseases, atopic and contact dermatitis, bronchiolitis, inflammatory muscle diseases, autoimmune peripheral neuropathies, immune renal, liver and thyroid diseases, inflammation and atherosclerosis, auto-inflammatory fever syndromes, immunohematology disorders and bullous diseases of skin and mucous membranes.

In one embodiment, the recombinant TCR recognizes GPC3, BCMA, GPRC5D, FAP, EGFR and mutants thereof, ASGPR1, mesothelin, CD19, IL-13RA2, CLDN18.2, CLL1, CS1, NGK2A, TIGIT, or CD94. In one embodiment, the recombinant TCR recognizes GPC3, GPRC5D, BCMA or NKG2A. In one embodiment, the recombinant TCR binds to the extracellular domain of the GPC3 polypeptide.

### 2.4 CD3 Complex

The recombinant TCR polypeptide provided by the present application is capable of associating with CD3ζ polypeptide. In one embodiment, the constant region of a TCR subunit in the recombinant TCR associates with a CD3ζ polypeptide. The CD3ζ polypeptide can be endogenous or exogenous. In one embodiment, the binding of the antigen-binding domain of the recombinant TCR with the antigen can activate the CD3ζ polypeptide associated with the constant region of the TCR subunit.

Activated CD3ζ polypeptide can activate and/or stimulate immune effector cells (e.g., cells of the lymphoid lineage, such as T cells). CD3ζ comprises three immunoreceptor tyrosine activation motifs (ITAM1, ITAM2, and ITAM3), and three basic-rich stretch regions (BRS) (BRS1, BRS2, and BRS3), and transmits an activation signal to cells (e.g., cells of the lymphoid lineage, such as T cells) after the antigen binds to the extracellular antigen binding domain of the recombinant TCR. The intracellular signaling domain of the CD3ζ chain is the main transmitter of TCR signals.

The recombinant TCR polypeptide provided by the present application can associate with the CD3 complex (also known as “T cell coreceptor”). In one embodiment, the complex of the recombinant TCR and CD3 forms an antigen recognition receptor complex similar to the natural TCR/CD3 complex. In one embodiment, the recombinant TCR can activate CD3 molecules associated with the recombinant TCR after binding to the antigen. CD3 molecules in the present application comprise CD3ζ, CD3γ, CD3δ and CD3ε.

CD3 complexes can be endogenous or exogenous. Recombinant TCR polypeptides replace the natural and/or endogenous TCR in the CD3/TCR complex. The CD3 complex comprises two CD3ζ, CD3γ chains, CD3δ chains and two CD3ε chains.

The recombinant TCR polypeptide provided by the present application shows higher antigen sensitivity than the CAR targeting the same antigen. In certain embodiments, recombinant TCRs are capable of inducing an immune response when binding to antigens with low density on the surface of tumor cells. In certain embodiments, immune effector cells comprising the recombinant TCR of the present application can be used to treat subjects with tumor cells that have low expression levels of surface antigens, such as due to relapse of disease, in which the subject has received treatment that resulted in residual tumor cells.

### 3. Engineered Cells

The engineered cells provided in the present application are immune effector cells carrying signal peptides that can promote high expression of the recombinant TCR. The engineered cells of the present application stably express the recombinant TCR. The engineered cells can significantly inhibit the growth of antigen-positive target cells.

Exemplarily, the engineered cells are T cells, also known as TCRT cells, which combine the high affinity and high specificity of the antibody/antigen-binding domain on antigen recognition with the natural TCR signaling ability of the T cells. The engineered cells of the present application show good killing effect on cells carrying antigens both in vivo and in vitro, and have significant advantages in the treatment of solid tumors.

In one embodiment, TCRT cells constructed with different signal peptides (such as signal peptides: GMCSFs, GMCSFRas, IgGsL1, IgGsL2, IgGsL3, IgGsL4, IgGsH1, IgGsH2, IgGsH3, and IgGsH4) are co-incubated with tumor cells expressing the target antigen, and the recombinant TCR positive rate of the TCRT cells is significantly increased. In one embodiment, the recombinant TCR positivity rate of the TCRT cells comprising synonymous mutated constant region is significantly increased compared to the wild-type constant region. In one embodiment, the positive rate of recombinant TCR in TCRT cells with hydrophobic amino acid mutations and/or cysteine point mutations in the constant region is significantly increased. In one embodiment, the TCRT of the present application exhibits comparable or better levels of engineered cell activation upon antigen-binding. It shows a good killing effect on cells carrying target antigens both in vivo and in vitro, and has significant advantages in the treatment of solid tumors.

In one embodiment, the engineered cells in the present application secrete anti-tumor cytokines. Cytokines secreted by the engineered cells include, but are not limited to, TNFα, IFNγ, and IL2. In one embodiment, the engineered cells of the present application exhibit a CD4/CD8 phenotype that is equivalent to or close to the natural state compared to cells comprising a CAR targeting the same antigen. In one embodiment, the engineered cells of the present application exhibit comparable or lower levels of exhaustion compared to cells comprising a CAR targeting the same antigen. In one embodiment, the engineered cells of the present application exhibit a proliferation ability that was equivalent to or closer to the natural state compared to cells comprising a CAR targeting the same antigen. In one embodiment, the engineered cells of the present application exhibit comparable or better therapeutic efficacy compared to cells comprising a CAR targeting the same antigen. In one embodiment, the engineered cells of the present application exhibit comparable or better cytolysis compared to cells comprising a CAR targeting the same antigen. In one embodiment, TCRT cells with low or no expression of endogenous TCR of the present application exhibit comparable or better anti-tumor effects compared to cells comprising a CAR targeting the same antigen.

In one embodiment, the TCRT cells described herein can further express another factor, such as a secreted or membrane-bound cytokine, a transcription factor, a chemokine, and/or a combination thereof, to increase proliferation, cell survival, anti-apoptotic effect, tumor infiltration and other effects of T cells to improve anti-tumor activity. In one embodiment, TCRT also expresses secreted or membrane-bound IL12. In one embodiment, TCRT also expresses IL15, IL18, IL21 and/or IL7.

In one embodiment, the coding sequence of the cytokine is placed under the control of a minimal promoter comprising an NFAT binding motif. In one embodiment, the IL2 minimal promoter comprising 6 NFAT binding motifs is a promoter consisting of 6 NFAT binding sites tandemly connected with the minimal promoter of IL2. In one embodiment, when the chimeric receptor recognizes the target antigen, the activated TCR signal can activate NFAT in the cell and bind to the NFAT binding motif in the promoter to initiate the transcription of the cytokine.

In one embodiment, in order to further improve the specificity of TCRT cells expressing cytokines, the endogenous TCR can also be knocked out through gene editing technology to eliminate the expression of cytokines induced by non-target antigens through the TCR/CD3 signaling pathway, and it is achieved that only the target antigen specifically induces TCRT cells to express cytokines, such as IL12.

In one embodiment, the IL2 minimal promoter comprising 6 NFAT binding motifs is a promoter consisting of 6 NFAT binding sites (SEQ ID NO: 37) tandemly connected with the IL2 minimal promoter (SEQ ID NO: 59), which can be used to regulate the expression of cytokines such as IL12 in T lymphocytes such as TCR-T.

In one embodiment, the recombinant TCR provided in the present application can form a complex with endogenous CD3 molecules. In one embodiment, the recombinant TCR can activate the CD3 molecule associated with the recombinant TCR after binding to the target antigen.

In one embodiment, the recombinant TCR amino acid sequence comprised in the engineered cells provided by the application comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the constant region of the wild-type TCR subunit, and the recombinant TCR polypeptide can form a complex with endogenous CD3. In one embodiment, the recombinant TCR nucleotide sequence comprised in the engineered cells provided by the application comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the constant region of the wild-type TCR subunit, and the recombinant TCR polypeptide can form a complex with endogenous CD3.

In one embodiment, the engineered cell positive rate of the recombinant TCR comprising IgGsH1 and IgGsL1, IgGsH1 and IgGsL2, IgGsH3 and IgGsL2, IgGsH2 and IgGsL1, IgGsH4 and IgGsL3, IgGsH4 and IgGsL4 signal peptides respectively connected with antigen-binding domains comprising VH and VL provided in the application is at least about 1, 1.1, 1.2, 1.3, 1.4, 1.5, 1.6, 1.7, 1.8, 1.9, 2, 2.2, 2.4, 2.5, 2.6, 2.8, 3, 3.5, 4, 4.5, or 5 times higher than the engineered cell positive rate of the recombinant TCR comprising TCR signal peptides or GMCSF signal peptides prepared in the same batch. In one embodiment, the engineered cells provided by the present application have low or no expression of endogenous TCR molecules, and the recombinant TCR comprised therein can form a complex with endogenous CD3. The recombinant TCR nucleic acid molecule is no longer the nucleic acid molecule of the target sequence targeted by gene knockout technology and/or gene silencing technology after comprising nucleotide mutations. In one embodiment, the recombinant TCR nucleic acid molecule is no longer the nucleic acid molecule of the target sequence targeted by gene knockout technology and/or gene silencing technology after comprising synonymous nucleotide mutations, and the recombinant TCR polypeptide can form a complex with the endogenous CD3. In one embodiment, the recombinant TCR nucleic acid molecule is no longer the nucleic acid molecule targeted by gene knockout technology and/or gene silencing technology after comprising synonymous nucleotide mutations, and the recombinant TCR amino acid sequence comprises an amino acid sequence or a fragment thereof having at least about 80%, at least about 85%, at least about 90%, at least about 95%, at least about 96%, at least about 97%, at least about 98%, at least about 99%, or at least about 100% homology or identity with the constant region of the wild-type TCR subunit, and the recombinant TCR polypeptide can form a complex with endogenous CD3.

In one embodiment, the engineered cells provided by the present application have low or no expression of endogenous αβTCR molecules, and the recombinant TCR nucleic acid molecules expressed comprise synonymous mutations relative to wild-type TRAC nucleic acid molecules and TRBC nucleic acid molecules. In one embodiment, the engineered cells provided by the present application have low or no expression of endogenous γδTCR molecules, and the recombinant TCR nucleic acid molecules expressed comprise synonymous mutations relative to wild-type TRGC nucleic acid molecules and TRDC nucleic acid molecules. In one embodiment, compared to the expression, activity and/or signaling of endogenous TCR in non-genetically engineered cells, the expression, activity and/or signaling of the endogenous TCR in the engineered cells is reduced by greater than about 50%, 60%, 70%, 80%, 90%, 95% or 100%. In one embodiment, CRISPR/Cas technology is used to knock out the exons of genes encoding the TCRα and β chain constant regions of the engineered cells. In one embodiment, the target sequences targeted by the CRISPR/Cas technology are located in the TCR α chain and β chain constant regions. In one embodiment, endogenous TRAC and endogenous TRBC are simultaneously knocked out in the engineered cells. In one embodiment, the engineered cells comprise gRNA with the sequence of SEQ ID NO: 49, 50, 51 or a combination thereof.

In one embodiment, compared to cells expressing the recombinant TCR carrying the same signal peptide, but the expression, activity and/or signaling of endogenous TCR subunits are not reduced or inhibited, the recombinant TCR positive rate of the engineered cells and/or the ratio of the complex formed by recombinant TCR and endogenous CD3 increases, or increases by about 5%, 10%, 20%, 30%, 35%, 40%, 45%, 50%, 55%, 60%, 65%, 70%, 75%, 80%, 85%, 90%, 95% or 100%.

The present application also provides TCRT cells, which are transduced with nucleic acid encoding the recombinant TCR, inhibitory nucleic acid molecules or gRNA targeting genes encoding endogenous TCR, or transduced with recombinant plasmids comprising the nucleic acids, or transduced with viruses comprising the plasmid. In one embodiment, TCRT cells are modified using two groups of polynucleotide fragments, wherein the first group of polynucleotide fragments comprises inhibitory nucleic acid molecules and/or gRNA, the complementary sequence of the inhibitory nucleic acid molecule or the gRNA target sequence is located in the constant region of the wild-type TCR subunit; the second group of nucleotide fragments comprises polynucleotide fragments encoding antigen binding domain recognizing antigens and the TCR subunit constant region comprising synonymous mutations in the polynucleotide sequence, and the second group of polynucleotide fragments does not comprise the complementary sequence of the inhibitory nucleic acid molecule and/or the gRNA target sequence in the first group of polynucleotide fragments.

The immune effector cells (also referred to as immune cells) described herein may be cells of the lymphoid lineage. The lymphoid lineage including B, T, and natural killer (NK) cells provides antibody production, regulation of the cellular immune system, detection of exogenous reagents in blood, detection of exogenous cells in the host, etc. Non-limiting examples of immune effector cells of the lymphoid lineage include T cells, natural killer T

(NKT) cells and their precursors, including embryonic stem cells and pluripotent stem cells (e.g., stem cells that differentiate into lymphoid cells or pluripotent stem cells). T cells can be lymphocytes that mature in the thymus and are primarily responsible for cell-mediated immunity. T cells participate in the adaptive immune system. T cells can be any type of T cell, including but not limited to helper T cells, cytotoxic T cells, memory T cells (including central memory T cells, stem cell-like memory T cells (or stem-like memory T cells), and two effector memory T cells: such as TEM cells and TEMRA cells), regulatory T cells (also called suppressor T cells), natural killer T cells, mucosal-associated invariant T cells, γδT cells, or αβT cells. Cytotoxic T cells (CTL or killer T cells) are T lymphocytes capable of inducing death of infected somatic cells or tumor cells. The subject's own T cells can be engineered to express the recombinant TCR to target specific antigens. In one embodiment, the immune effector cells are T cells. In one embodiment, the T cells can be CD4+ T cells and/or CDδ+ T cells. In one embodiment, the immune effector cells are CD3+ T cells. In one embodiment, the engineered cells comprise a cell population collected from PBMC cells after stimulation with CD3 magnetic beads.

Immune effector cells (e.g., T cells) can be autologous, non-autologous (e.g., allogeneic), or derived from engineered progenitor or stem cells in vitro. It can be obtained from a number of sources, including peripheral blood mononuclear cells (PBMC), bone marrow, lymph node tissue, umbilical cord blood, thymus tissue, tissue from the infected site, ascites, pleural effusion, spleen tissue, and tumors.

In certain aspects of the present application, T cells can be obtained from a blood sample collected from a subject using any number of techniques known to those of skill in the art, such as the Ficoll™ separation technique. In a preferred aspect, cells from the circulating blood of the individual are obtained by apheresis. Products of apheresis usually comprise lymphocytes, comprising T cells, monocytes, granulocytes, B cells, other nucleated white blood cells, red blood cells, and platelets. In one aspect, cells collected by apheresis may be washed to remove the plasma fraction and placed in an appropriate buffer or culture medium for subsequent processing steps. Multiple rounds of selection may also be used in the context of the present application. In some aspects, it may be necessary to perform a selection procedure and use “unselected” cells during activation and expansion. “Unselected” cells can also undergo selection of other rounds.

The engineered cells of the present application are capable of regulating the tumor microenvironment.

The source of unpurified CTL can be any source known in the art, such as bone marrow, fetal, neonatal or adult or other source of hematopoietic cells, such as fetal liver, peripheral blood or umbilical cord blood. Cells can be isolated using various techniques. For example, non-CTLs can be initially removed by negative selection. mAb is particularly useful for identifying markers associated with specific cell lineages and/or differentiation stages of positive and negative selection.

Most of the terminally differentiated cells can be removed initially by relatively rough separation. For example, magnetic bead separation may be used initially to remove large numbers of irrelevant cells. In certain embodiments, at least about 80%, usually at least about 70%, of the total hematopoietic cells will be removed prior to isolating the cells. Separation procedures include, but are not limited to, density gradient centrifugation; resetting; coupling to particles altering cell density; magnetic separation with antibody-coated magnetic beads; affinity chromatography; using cytotoxic agents combined with or in combination with mAb, including but not limited to complement and cytotoxins; and panning with antibodies attached to a solid substrate (e.g., plate, chip, elutriation) or any other convenient technique.

Techniques for separation and analysis include, but are not limited to, flow cytometry, which can have varying degrees of sophistication, such as multiple color channels, low- and obtuse-angle light-scattering detection channels, and impedance channels.

Cells can be selected from dead cells by using dyes associated with dead cells, such as propidium iodide (PI). In certain embodiments, cells are harvested in medium comprising 2% fetal calf serum (FCS) or 0.2% bovine serum albumin (BSA), or any other suitable medium such as sterile isotonic medium.

Quantification of recombinant TCR-positive engineered cells may include, but is not limited to, ELISA assay, Western blot, immunoprecipitation, immunofluorescence, mass spectrometry, flow cytometry, or fluorescence-activated cell sorting (FACS). The method allows determining which signal peptides are able to promote maximal expression and secretion of the recombinant TCR.
4. Vector

Genetic modification of engineered cells (e.g., T cells or NKT cells) can be accomplished by transducing a substantially homogeneous population of cells with recombinant DNA molecules. In certain embodiments, retroviral vectors (y-retrovirus or lentivirus) are used to introduce DNA molecules into cells. For example, a polynucleotide encoding the recombinant TCR can be cloned into a retroviral vector. Non-viral vectors can also be used. Transduction can use any suitable viral vector or non-viral delivery system. Recombinant TCRs can be constructed with accessory molecules (e.g., cytokines) in a single polycistron expression cassette, multiple expression cassettes in a single vector, or multiple vectors. Examples of elements that generate polycistron expression cassettes include, but are not limited to, various viral and non-viral internal ribosome entry sites (IRES, e.g., FGF-1 IRES, FGF-2 IRES, VEGF IRES, IGF-II IRES, NF-κB IRES, RUNX1 IRES, p53IRES, hepatitis A IRES, hepatitis C IRES, pestivirus IRES, nonbaculovirus IRES, picornavirus IRES, poliovirus IRES, and encephalomyocarditis virus IRES) and cleavable linkers (e.g., 2A peptides such as P2A, T2A, E2A and F2A peptides).

Other viral vectors that may be used include, for example, adenovirus, lentiviral and adeno-associated viral vectors, vaccinia virus, bovine papillomavirus or herpesviruses, such as Epstein-Barr virus.

Non-viral methods can also be used for genetic modification of engineered cells. For example, nucleic acid molecules can be introduced into immune effector cells by administering the nucleic acid in the context of lipofection, asialooorosomucoid-polylysine conjugation, or microinjection under surgical conditions. Other non-viral gene transfer methods comprise in vitro transfection using liposomes, calcium phosphate, DEAE dextran, electroporation and protoplast fusion. Transplantation of the nucleic acid molecule into a subject can also be accomplished by transferring the nucleic acid molecule into a cell type that can be cultured ex vivo (e.g., autologous or allogeneic primary cells or their progeny), after which the cells (or their progeny) modified by the nucleic acid molecules are injected into the target tissue of the subject or injected systemically.

Gene knockout technology and/or gene silencing technology are used to prepare engineered cells with low or no expression of endogenous TCR molecules. Gene knockout technologies comprise Argonaute, CRISPR/Cas9 technology, ZFN technology, TALE technology, TALE-CRISPR/Cas9 technology, Base Editor technology, guided editing technology and/or meganuclease technology. Gene silencing technologies include, but are not limited to: antisense RNA, RNA interference, microRNA-mediated translation inhibition, etc.

The clustered regularly interspaced short palindromic repeats (CRISPR) system is used for genome editing. The system comprises Cas9 (a protein capable of modifying DNA using crRNA as its guide), CRISPR RNA (crRNA, which comprises the RNA that Cas9 uses to guide it to the correct segment of host DNA), and a region that binds to tracrRNA (usually in the form of hairpin ring and forms an active complex with Cas9), transactivating crRNA (tracrRNA, binds to crRNA, and forms an active complex with Cas9), and an optional fragment of the DNA repair template (DNA that directs the cellular repair process to allow insertion of specific DNA sequence). CRISPR/Cas9 usually uses plasmids or electroporation to deliver nucleic acid fragments to target cells. The crRNA needs to be designed for each application because this is the sequence Cas9 uses to recognize and directly bind to target DNA in the cell. Multiple crRNA and tracrRNA can be packaged together to form guide RNA (gRNA). The gRNA can be connected with the Cas9 gene and form a plasmid for transfection into cells. Whenever the gRNA sequence is involved in the present application, it can be a targeted DNA sequence, or it can be a complete Cas9 guide sequence formed by the ribonucleotide corresponding to the DNA, crRNA, and TracrRNA. When performing gene editing, the gRNA, tracr pairing sequence and tracr sequence used can be used individually or as a whole RNA sequence. CRISPR/Cas9 transgenes can be delivered via vectors (e.g., AAV, adenovirus, lentivirus), and/or particles and/or nanoparticles, and/or electroporation.

Zinc finger nuclease (ZFN) is an artificial restriction enzyme produced by binding a zinc finger DNA-binding domain with a DNA cleavage domain. Zinc finger domains can be engineered to target specific DNA sequences, which allow zinc finger nucleases to target target sequences within the genome.

Transcription activator-like effector nucleases (TALENs) are restriction enzymes that can be engineered to cleave specific sequences of DNA. The working principle of the TALEN system is almost same as that of ZFN. They are generated by binding the DNA-binding domain of a transcription activator-like effector to a DNA cleavage domain.

The present application also provides nucleic acid molecules encoding one or more recombinant TCRs described herein, nucleic acid inhibitory molecules or gRNA molecules targeting endogenous TCRs.

### 5. Preparation Methods of Engineered Cells

In one embodiment, immune effector cells (e.g., T cells or NKT cells) are infected with a virus comprising the polynucleotide encoding the recombinant TCR. In one embodiment, a virus comprising the polynucleotide encoding the recombinant TCR is first used to infect immune effector cells (such as T cells or NKT cells), and then CRISPR/Cas9 technology is used to knock out the endogenous TCR subunits to obtain the engineered cells of the present application. In one embodiment, CRISPR/Cas9 technology is first used to knock out the endogenous TCR subunits of immune effector cells, and then infected with a virus comprising the polynucleotide encoding the recombinant TCR. In one embodiment, a virus comprising the polynucleotide encoding the recombinant TCR is used to infect immune effector cells and CRISPR/Cas9 technology is used to knock out endogenous TCR subunits of the immune effector cells simultaneously. In one embodiment, the polynucleotide fragment encoding the recombinant TCR does not comprise the target sequence of CRISPR/Cas9.

In one embodiment, in order to reduce the mismatch between endogenous TCR subunits and recombinant TCR subunits in T cells, resulting in low expression of the recombinant TCR, the present application knocks out the endogenous TCR of T cells or genetically modifies T cells to get low expression or no expression of endogenous TCR molecules, and the extracellular constant region of the TCR subunit in the recombinant TCR is genetically modified, so that knocking out endogenous TCR in T cells or performing gene modifications to get low or no expression of endogenous TCR molecules does not affect the expression of the recombinant TCR in T cells and/or does not affect formation of the complexes between the recombinant TCR and endogenous CD3 in T cells.

In one aspect, nucleic acid molecules encoding different signal peptides and the recombinant TCR targeting a target antigen (exemplarily, GPC3 tumor antigen), nucleic acid inhibitory molecules or gRNA targeting endogenous TCR, are introduced into T cells to generate TCRT cells. In one embodiment, in vitro transcribed recombinant TCR nucleic acid molecules carrying different signal peptides, nucleic acid inhibitory molecules or gRNA targeting endogenous TCR, can be introduced into cells as a transient transfection form. An exemplary artificial DNA sequence is a sequence comprising portions of a gene connected together to form an open reading frame encoding a fusion protein. The DNA portions connected together can be from a single organism or from more than one organism.

### 6. Administration

Compositions comprising the engineered cells of the present application can be provided to a subject systemically or directly to induce and/or enhance an immune response to an antigen and/or to treat and/or prevent tumors, pathogenic infections, or infectious diseases. In one embodiment, the engineered cells of the present application or compositions comprising the same are injected directly into the organ of interest (e.g., an organ affected by a tumor). Alternatively, the engineered cells of the present application or compositions comprising the same are provided to the organ of interest indirectly, such as by administering into the circulatory system (e.g., veins, tumor vasculature). Expansion and differentiation agents can be provided before, simultaneously, or after administration of the cells or composition to increase production of T cells, NKT cells, or CTL cells in vitro or in vivo.

The engineered cells of the present application may comprise purified cell populations. One skilled in the art can readily determine the percentage of engineered cells of the present application in a population using a variety of well-known methods, such as fluorescence-activated cell sorting (FACS). In a population comprising the engineered cells of the present application, suitable ranges for purity are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosage can be readily adjusted by one skilled in the art (e.g., reduced purity may require increased dosage). Cells can be introduced by injection, catheter, etc.

The composition of the present application may be a pharmaceutical composition comprising the immune effector cells of the present application or their progenitor cells and a pharmaceutically acceptable carrier. Administration may be autologous or allogeneic. For example, immune effector cells or progenitor cells can be obtained from one subject and administered to the same subject or to a different compatible subject. Peripheral blood-derived immune effector cells or their progeny (e.g., from in vivo, ex vivo, or in vitro sources) can be administered by local injection, including catheter administration, systemic injection, local injection, intravenous injection, or parenteral administration. When administering a therapeutic composition of the subject of the present application (e.g., a pharmaceutical composition comprising the immune effector cell of the present application), it may be formulated in a unit dose injectable form (solution, suspension, emulsion, etc.).

### 7. Dosage Form

Compositions comprising the engineered cells of the present application may be provided conveniently in the form of sterile liquid preparations, such as isotonic aqueous solutions, suspensions, emulsions, dispersions or viscous compositions, which may be buffered to a selected pH value. Liquid formulations are generally easier prepared than gels, other viscous compositions, and solid compositions. Additionally, liquid compositions are somewhat more convenient for administration, especially by injection. On the other hand, viscous compositions can be formulated within an appropriate viscosity range to provide longer contact time with a specific tissue. Liquid or viscous compositions may comprise a carrier, which may be a solvent or dispersion medium, comprising, for example, water, saline, phosphate-buffered saline, polyols (e.g., glycerol, propylene glycol, liquid polyethylene glycol, etc.), and a suitable mixture thereof.

Sterile injectable solutions can be prepared by incorporating the genetically modified engineered cells in required amount of appropriate solvent, and incorporating different amounts of other ingredients as required. Such composition may be mixed with suitable carriers, diluents or excipients such as sterile water, physiological saline, glucose, dextrose and the like. The composition may also be lyophilized. The composition may comprise auxiliary substances such as wetting, dispersing or emulsifying agents (e.g., methylcellulose), pH buffering agents, gelling or viscosity-increasing agents, preservatives, flavoring agents, pigments, etc., depending on the administration route and desired formulation.

Various additives, including antimicrobial preservatives, antioxidants, chelating agents, and buffering agents, may be added to enhance the stability and sterility of the compositions. Preventions of actions of microorganisms can be ensured by various antibacterial and antifungal agents, for example, parabens, chlorobutanol, phenol, sorbic acid, and the like. Absorption of the injectable pharmaceutical forms can be prolonged by the use of agents which delay absorption, for example, aluminum monostearate and gelatin. However, any vehicle, diluent or additive used will have to be compatible with the genetically modified immune effector cells or progenitors thereof.

The compositions may be isotonic, i.e., may have the same osmotic pressure as blood and/or tear. The desired isotonicity of the compositions can be achieved by using sodium chloride or other pharmaceutically acceptable agent, such as dextrose, boric acid, sodium tartrate, propylene glycol or other inorganic or organic solutes. Sodium chloride may be particularly suitable for buffers containing sodium ions.

If desired, a pharmaceutically acceptable thickener may be used to maintain the viscosity of the composition at a selected level. For example, methylcellulose is readily and economically available and easy to use. Other suitable thickeners include, for example, xanthan gum, carboxymethylcellulose, hydroxypropylcellulose, carbomer, and the like. The concentration of the thickener can depend on the reagent chosen. It is important to use the amount that will achieve the chosen viscosity. Obviously, the selection of suitable carriers and other additives will depend on the exact administration route and the nature of the specific dosage form, e.g., liquid dosage form (e.g., whether the composition is formulated as a solution, suspension, gel, or other liquid form, e.g. timed-release or liquid-filled form).

The number of cells to be administered will vary for the subject being treated. The precise determination of an effective dose can be determined according to each subject's individual factors, including size, age, sex, weight and status of the subject. Dosages can be readily determined by those skilled in the art from the present application and knowledge in the art.

Those skilled in the art can readily determine the amount of cells and optional additives, vehicles and/or carriers in the composition and to be administered in the method. Typically, any additive (other than one or more active cells and/or one or more reagents) is present in 0.001% to 50% by weight of the solution in phosphate-buffered saline, and the active ingredient is present in order of micrograms to milligram, for example, about 0.0001 wt % to about 5 wt %, about 0.0001 wt % to 1 wt %, about 0.0001 wt % to about 0.05 wt %, or about 0.001 wt % to about 20 wt %, about 0.01 wt % to about 10 wt % % or about 0.05 wt % to about 5 wt %. For any composition to be administered to animals or humans, the following results may be determined: toxicity, for example by determining the lethal dose (LD) and LD50 in a suitable animal model, e.g. rodents such as mice; dose of the composition, wherein concentration of the ingredients and application time of the composition elicit an appropriate response.

### 8. Treatment Methods

The present application provides a method for inducing and/or increasing an immune response in a subject in need of the engineered cells. The engineered cells of the present application and compositions comprising the same can be used to treat and/or prevent tumors in a subject. The engineered cells of the present application and compositions comprising the same can be used to extend survival of subjects suffering from tumors. The engineered cells of the present application and compositions comprising the same may also be used to treat and/or prevent pathogenic infections or other infectious diseases, such as in immunocompromised human subjects. The engineered cells of the present application and compositions comprising the same can be used to resist transplant immune rejection, and particularly relate to a method for resisting NK cell immune rejection. The engineered cells of the present application and compositions comprising the same can be used to treat, prevent or improve autoimmune diseases or inflammatory diseases, especially inflammatory diseases related to autoimmune diseases. Such methods comprise administering an effective amount of the engineered cells of the present application or a composition (e.g., a pharmaceutical composition) comprising the same to achieve a desired effect, whether alleviating an existing condition or preventing recurrence. For treatment, the amount administered is an amount effective to produce the desired effect. An effective amount may be provided in one or more administrations. Effective amounts can be provided in large doses or by continuous infusion.

In one embodiment, immune effector cells comprising recombinant TCRs of the present application can be used to treat a subject with tumor cells with low levels of surface antigen expression, for example, due to relapse of the disease, wherein the subject has received treatments that result in residual tumor cells. In certain embodiments, tumor cells have a low density of target molecules on the tumor cell surface.

In one embodiment, the immune effector cells comprising the recombinant TCR of the present application can be used to treat a subject suffering from disease relapse, wherein the subject has received immune effector cells (e.g., T cells) comprising CAR, wherein the CAR comprises an intracellular signaling domain comprising a costimulatory signaling domain (e.g., 4-1BBz CAR). In certain embodiments, tumor cells have a low density of tumor specific antigens on the surface. In one embodiment, the disease is a GPC3-positive tumor. In one embodiment, the disease is a BCMA-positive tumor. In one embodiment, the disease is a GPRC5D positive tumor. In one embodiment, the tumor cells have a low density of GPC3. Such methods comprise administering an effective amount of the immune effector cells of the present application or a composition (e.g., a pharmaceutical composition) comprising the same to achieve the desired effect, alleviate an existing condition, or prevent recurrence.

An “effective amount” (or “therapeutically effective amount”) is an amount sufficient to produce beneficial or desired clinical results following treatment. The effective amount may be administered to a subject in one or more doses. For treatment, the effective amount is an amount sufficient to alleviate, ameliorate, stabilize, reverse or slow the progression of a disease or otherwise reduce the pathological consequences of the disease. The effective amount is generally determined by a physician on a case-by-case basis and is within the competence of those skilled in the art. Several factors are generally considered when determining a suitable dosage to achieve the effective amount. These factors comprise the age, sex and weight of the subject, the disease to be treated, the severity of the disease, and the form and effective concentration of the immune effector cells of the present application to be administered.

After the engineered cells of the present application are administered to a host and subsequently differentiated, T cells specific for a particular antigen are induced. Engineered cells may be administered by any method known in the art, including, but not limited to, intravenous, subcutaneous, intranodal, intratumoral, intrathecal, intrapleural, intraperitoneal administration, and direct administration to the thymus.

The present application provides methods for treating and/or preventing tumors in a subject. The method may comprise administering to a subject suffering from a tumor an effective amount of the engineered cells of the present application or the composition comprising the same.

Non-limiting examples of tumors include blood cancers (such as leukemia, lymphoma, and myeloma), ovarian cancer, breast cancer, bladder cancer, brain cancer, colon cancer, intestinal cancer, liver cancer, lung cancer, pancreatic cancer, prostate cancer, skin cancer, gastric cancer, glioblastoma, laryngeal cancer, melanoma, neuroblastoma, adenocarcinoma, glioma, soft tissue sarcomas, and various carcinomas (including prostate carcinoma and small cell lung carcinoma). Non-limiting examples of tumors include, but are not limited to, astrocytoma, fibrosarcoma, myxosarcoma, liposarcoma, oligodendroglioma, ependymoma, medulloblastoma, primitive neuroectodermal tumor (PNET), chondrosarcoma, osteosarcoma, pancreatic ductal adenocarcinoma, small and large cell lung adenocarcinoma, chordoma, angiosarcoma, endothelial sarcoma, squamous cell carcinoma, bronchoalveolar carcinoma, epithelial adenocarcinoma and its liver metastases, lymphatic sarcoma, lymphangioendothelial sarcoma, liver cancer, cholangiocarcinoma, synovial tumor, mesothelioma, Ewing's sarcoma, rhabdomyosarcoma, colon cancer, basal cell carcinoma, sweat gland carcinoma, papillary carcinoma, sebaceous gland carcinoma, thyroid carcinoma, cyst gland carcinoma, medullary carcinoma, bronchial carcinoma, renal cell carcinoma, cholangiolar carcinoma, choriocarcinoma, seminoma, embryonal carcinoma, Wilms' tumor, testicular tumor, medulloblastoma, craniopharyngioma, ependymoma, pineal tumor, hemangioblastoma, acoustic neuroma, oligodendroglioma, meningioma, neuroblastoma, retinoblastoma, leukemia, multiple myeloma, Waldenstrom's macroglobulinemia and heavy chain diseases, breast tumors such as ductal and lobular adenocarcinomas, squamous and adenocarcinomas of the cervix, epithelial carcinomas of the uterus and ovaries, prostate adenocarcinomas, transitional squamous cell carcinoma of the bladder, B and T-cell lymphomas (nodular and diffuse) plasmacytoma, acute and chronic leukemia, malignant melanoma, soft tissue sarcoma, and leiomyosarcoma. In certain embodiments, the tumor is selected from the group consisting of: blood cancers (e.g., leukemia, lymphoma, and myeloma), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In one embodiment, engineered cells of the present application or a composition comprising the same may be used for the treatment and/or prevention of conventional treatment unsuitable or relapsed refractory solid tumors, such as liver cancer, lung cancer, breast cancer, ovarian cancer, kidney cancer, thyroid cancer, gastric cancer, colorectal cancer. In one embodiment, the tumor is a hematological tumor.

The therapeutic goals of the engineered cell of the present application may comprise alleviating or reversing disease progression and/or alleviating side effects, or the therapeutic goals may comprise reducing or delaying the risk of relapse.

The present application provides a method for treating and/or preventing a pathogen infection (e.g., viral, bacterial, fungal, parasitic, or protozoan infection) in, e.g., an immunocompromised subject. The method may comprise administering an effective amount of the engineered cells of the present application or the composition comprising the same to a subject suffering from the pathogenic infection. Exemplary viral infections that are amenable to treatment include, but are not limited to, cytomegalovirus (CMV), Epstein-Barr virus (EBV), human immunodeficiency virus (HIV), and influenza virus infections.

The term “enhancement” refers to the ability which allows a subject or a tumor cell to improve the response to the treatments disclosed herein. For example, enhanced response can comprise 5%, 10%, 15%, 20%, 25%, 30%, 35%, 40%, 45%, 50%, 55%, 60%, 65%, 70%, 75%, 80%, 85%, 90%, 95%, or 98% or more increase in responsiveness. As used herein, “enhancement” may also refer to increasing the number of subjects responding to treatment, e.g., immune effector cell therapy. For example, enhanced response may refer to the total percentage of subjects who respond to treatment, wherein the percentage is 5%, 10%, 15%, 20%, 25%, 30%, 35%, 40%, 45%, 50%, 55%, 60%, 65%, 70%, 75%, 80%, 85%, 90%, 95%, or 98% or more.

In the embodiments of the present application, immune effector cells target GPC3-positive tumors. In specific embodiments, the tumors include, but are not limited to, liver cancer, gastric cancer, lung cancer, esophageal cancer, head and neck cancer, bladder cancer, ovarian cancer, cervical cancer, kidney cancer, pancreatic cancer, cervical cancer, liposarcoma, melanoma, adrenal carcinoma, schwannoma, malignant fibrous histiocytoma, and esophageal cancer. Those skilled in the art know that some tumor cells, such as liver cancer cells, are insensitive to many drugs. Therefore, even drugs that are effective in vitro sometimes have poor or even no effect in vivo. Therefore, in a preferred embodiment, the GPC3-positive tumors described herein include, but are not limited to, liver cancer, gastric cancer, lung cancer, and esophageal cancer.

### 9. Kit

The present application provides a kit for inducing and/or enhancing immune responses and/or treating and/or preventing tumors or pathogenic infections in a subject. In one embodiment, the kit comprises an effective amount of the engineered cells of the present application or a pharmaceutical composition comprising the same. In one embodiment, the kit comprises a sterile container; such container may be in the form of a box, ampoule, bottle, vial, tube, bag, sachet, blister pack, or other suitable container known in the art. Such containers may be made of plastic, glass, laminated paper, metal foil, or other materials suitable for accommodate the drug. In one embodiment, the kit comprises a nucleic acid molecule encoding the recombinant TCR of the present application, which targets the antigen of interest in an expressible form and may optionally be comprised in one or more vectors.

In one embodiment, the engineered cells and/or nucleic acid molecules of the present application are provided with an instruction for administering the cell or nucleic acid molecule to a subject suffering from or developing a tumor, pathogen, or immune disease. Instructions typically comprise information regarding the use of the composition to treat and/or prevent tumors or pathogenic infections. In one embodiment, the instruction comprises at least one of the following: description of the therapeutic agent; a dosage schedule and administration for treating or preventing a tumor, pathogenic infection, or immune disease, or symptoms thereof; precautions; warnings; indications; inadaptable syndrome; medication information; adverse reactions; animal pharmacology; clinical studies; and/or references. These instructions may be printed directly on the container, or as a label affixed to the container, or provided as a separate sheet, booklet, card or folder within or with the container.

### EXAMPLES

The present application is described in further detail with reference to the following experimental examples. These examples are provided for illustrative purposes only and are not intended to be limiting unless otherwise stated. Accordingly, the present application should in no way be construed as limited to the following examples, but should be construed to cover any and all changes that become apparent as a result of the teachings provided herein.

### Example 1. Construction of Recombinant TCRs Carrying Different Antibody Signal Peptides

The present application provides recombinant TCRs comprising different antibody signal peptides:
(1) Chain one: antibody heavy chain signal peptide, antibody VH, TRAC; chain two: antibody light chain signal peptide, antibody VL, TRBC; or
(2) Chain one: antibody light chain signal peptide, antibody VL, TRAC; chain two: antibody heavy chain signal peptide, antibody VH, TRBC.

Exemplarily, different combinations of antibody light chain signal peptides and antibody heavy chain signal peptides are shown in Table 1.

TABLE 1
Combinations of different signal peptides

| | signal peptide connected | signal peptide connected |
|---|---|---|
| | with antibody VH | with antibody VL |
| combination 1 | IgGsH2 | IgGsL1 |
| combination 2 | IgGsH1 | IgGsL1 or IgGsL2 |
| combination 3 | IgGsH3 | IgGsL1 or IgGsL2 |
| combination 4 | IgGsH4 | IgGsL3 |
| combination 5 | IgGsH4 | IgGsL4 |

### Example 2. Construction of GPC3-Recombinant TCRs Carrying Different Signal Peptides

Exemplary, recombinant TCRs targeting the tumor antigen GPC3 and comprising different signal peptides and natural TCR subunit polypeptides:
TCRs-GPC3-TCR fragment: comprising in sequence TRAVs (SEQ ID NO: 2), GPC3 antibody VH, TRAC nucleic acid fragment 1, P2A, TRBVs (SEQ ID NO: 4), GPC3 antibody VL, and TRBC nucleic acid fragment 1;
GMCSFs-GPC3-TCR fragment: comprising in sequence GMCSFs (SEQ ID NO: 6), GPC3 antibody VH, TRAC nucleic acid fragment 1, F2A, GMCSF signal peptide (SEQ ID NO: 6), GPC3 antibody VL, and TRBC nucleic acid fragment 1;
GMCSFRas-GPC3-TCR fragment: comprising in sequence GMCSFRas (SEQ ID NO: 8), GPC3 antibody VH, TRAC nucleic acid fragment 1, F2A, GMCSFRas signal peptide (SEQ ID NO: 8), GPC3 antibody VL, and TRBC nucleic acid fragment 1;
IgGs-GPC3-TCR fragment: comprising in sequence IgGsH1 (SEQ ID NO: 15), GPC3 antibody VH, TRAC nucleic acid fragment 1, P2A, IgGsL1 (SEQ ID NO: 10), GPC3 antibody VL, and TRBC nucleic acid fragment 1;
The above recombinant TCR fragments were constructed respectively in lentiviral vectors. Among them, GPC3 antibody VH (SEQ ID NO: 38), GPC3 antibody VL (SEQ ID NO: 39), TRAC nucleic acid fragment 1 (SEQ ID NO: 25), TRBC nucleic acid fragment 1 (SEQ ID NO: 32), P2A (SEQ ID NO: 46), F2A (SEQ ID NO: 47).

### Example 3. Detection of Positive Rate of GPC3-Recombinant TCR Carrying Different Signal Peptides in Jurkat and J.RT-T3.5 Cells

The viruses comprising the vectors IgGs-GPC3-TCR, TCRs-GPC3-TCR, GMCSFs-GPC3-TCR or GMCSFRas-GPC3-TCR constructed in Example 2 were used to infect Jurkat and J.RT-T3.5 cells (TCR-β deleted mutant Jurkat cell line, CD3 negative), respectively. On days 4, 7, 11, 16, and 19 after infection, flow cytometry was used to detect the positive rate. The detection reagent was 5 μg/ml biotinylated human GPC3 protein, and then SA-PE fluorescent antibody (eBioscience) with 1:300 dilution was added for labeling; at the same time, Anti-CD3-BV421 (BD) was used to detect the expression of CD3 on the cell surface, and the ratio of CD3 and GPC3 double positivity was analyzed.

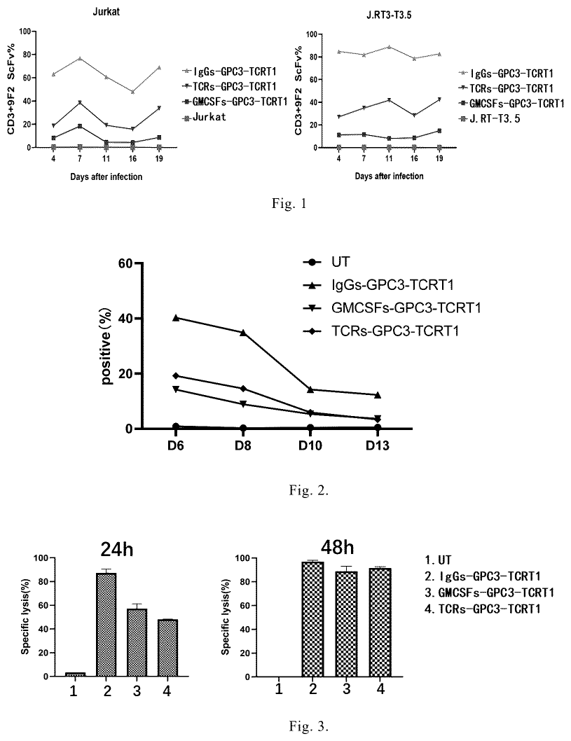

Flow cytometry results indicate that recombinant TCRs constructed with different signal peptides can form recombinant TCR/CD3 complexes with endogenous CD3. Jurkat cells and J.RT-T3.5 cells were infected respectively, and the positive rates of recombinant TCRs constructed with different signal peptides are shown in FIG. 1.

### Example 4. Preparation of GPC3-TRCT Cells Carrying Different Signal Peptides

T cells were isolated and prepared from the blood of healthy donors using conventional biological means. After resuscitation and activation of the T cells, viruses comprising vectors IgGs-GPC3-TCR, TCRs-GPC3-TCR, GMCSFs-GPC3-TCR or GMCSFRas-GPC3-TCR was added for infection to obtain IgGs-GPC3-TCRT1, TCRs-GPC3-TCRT1, GMCSFs-GPC3-TCRT1, and GMCSFRas-GPC3-TCRT1 cells. The UT group was T cells uninfected by virus. The positive rate of recombinant TCR expression was detected at different time points of T cell activation (FIG. 2 and Table 2): the positive rate of recombinant TCRs constructed with different signal peptides from high to low was IgGs, GMCSFs and TCRs.

TCRT cells after lentivirus infection were collected, and lysed using protein lysis buffer, using biotin-labeled GPC3 antigen, streptavidin-labeled magnetic beads to co-immunoprecipitate the recombinant TCR and its binding protein, performing western-blot experiment, and using corresponding antibodies to detect the expression of different CD3 subunits. The results show that recombinant TCRs carrying different signal peptides can form complexes with CD3, CD3γ, CD3δ and CD3E.

TABLE 2
positive rate detection of the recombinant TCR

| | positive | positive | positive | positive |
|---|---|---|---|---|
| | rate on | rate on | rate on | rate on |
| cell | D 6 | D 8 | D 10 | D 13 |
| UT | 0.2% | 0.3% | 0.45% | 0.54% |
| TCRs-GPC3-TCRT1 | 14.2% | 8.91% | 5.34% | 3.65% |
| GMCSFs-GPC3-TCRT1 | 19.2% | 14.6% | 5.94% | 3.38% |
| IgGs-GPC3-TCRT1 | 40.3% | 34.9% | 14.3% | 12.3% |

### Example 5. GPC3-TCRT Carrying Different Signal Peptides Kills Liver Cancer Cells In Vitro

Target cells: PLC/PRF/5 cells expressing GPC3 (ATCC, USA); effector cells: UT, IgGs-GPC3-TCRT1, TCRs-GPC3-TCRT1, and GMCSFs-GPC3-TCRT1.

The xCELLigence RTCA Instrument (Agilent Company) was used for detection. 100 μL of 1×10⁵/mL target cells were inoculated onto the corresponding 96-well electrode plates, and the plates were placed on the xCELLigence RTCA Instrument. 24 hours after target cells inoculated, effector cells were inoculated at an effector-to-target ratio of 1:1. Two duplicate wells were set up in each group to continuously observe the effect of effector cells on proliferation of target cells. The settings of each experimental group and each control group were as follows: experimental group: target cells+different effector cells; control group: target cells. The calculation formula is: % cytotoxicity=(1−NCI experimental group/NCI control group)*100. (NCI: Normalized Cell Index). The results show that compared to UT, TCRT cells constructed with different signal peptides can significantly kill target cells after co-incubation for 24 or 48 hours (FIG. 3).

### Example 6. Knocking Out Endogenous TCR in T Cells

Since endogenous TCR will compete for the expression of exogenous TCR or cause mismatching, which will affect the expression of recombinant TCR on the cell surface, CRISPR technology was used to knock out the endogenous TCR. Exemplarily, the sequences of the gRNA-TRAC and gRNA-TRBC used were as shown in SEQ ID NO: 49 and 50, respectively. Using conventional molecular biology techniques in the field, the Cas 9 enzyme, the nucleic acid comprising the gRNA targeting TRAC, and the nucleic acid comprising the gRNA targeting TRBC were co-electroporated to T cells to obtain T cells with endogenous TCR a chain and β chain knockout.

### Example 7. Construction of the Recombinant TCR with Synonymous Mutations in the TCR Constant Region

In order to prevent the knockout of the endogenous TCR in Example 6 from affecting the exogenous TCR, the nucleotides of the constant region of the TCR in the recombinant TCR were mutated, and the amino acid sequence of the mutated recombinant TCR remained unchanged. Exemplarily, the TRAC and/or TRBC sequences targeted by the gRNA in Example 6 were synonymously mutated, as shown in Table 3.

TABLE 3
mutation sites in the constant region of exogenous TCR α chain and β
chain (underlined)

| position | original sequence | mutated sequence |
|---|---|---|
| α chain constant | TGTACCAGCTGAGAGACTCT | TGTATCAGCTGAGGGATTCC |
| region | | |
| β chain constant | AGATCGTCAGCGCCGAGGCC | AGATTGTCTCCGCCGAAGCC |
| region | | |

Exemplarily, recombinant TCR that targets GPC3 and comprises different signal peptides and synonymous mutations in the TCR constant region was constructed referring to Example 2.

### Example 8. Preparation of GPC3-TCRT2 Cells with Endogenous TCR Knockout

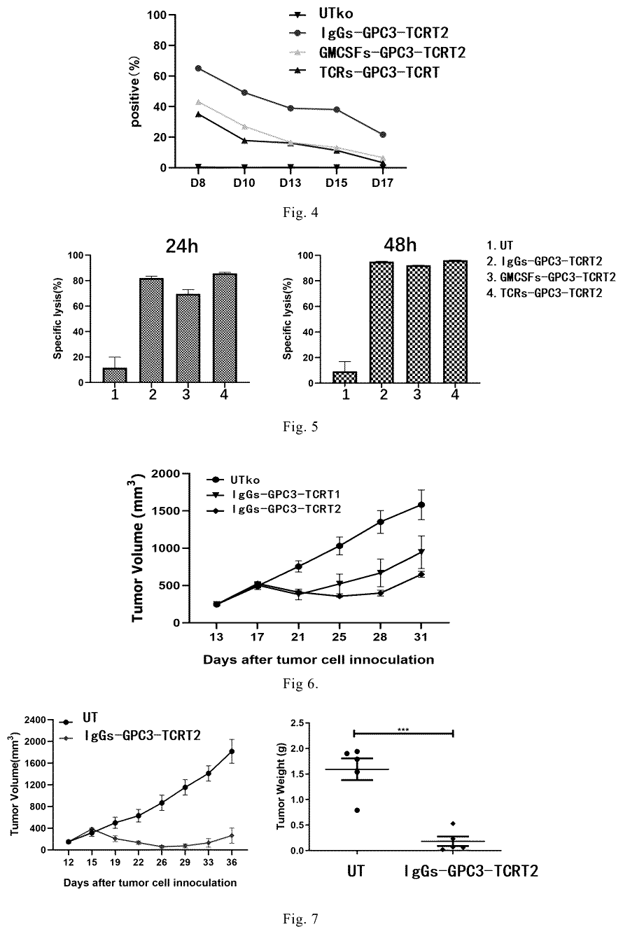

T cells were isolated and prepared from the blood of healthy donors using conventional biological means. T cells were resuscitated and activated for 24-48 hours, and the viruses comprising the recombinant TCR vectors were added respectively. 24-96 hours after infection, the CRISPR/Cas9 technology described in Example 6 was used to knock out the TCR a chain and β chain in the cells to obtain IgGs-GPC3-TCRT2, TCRs-GPC3-TCRT2, GMCSFs-GPC3-TCRT2, and GMCSFRas-GPC3-TCRT2 cells with the endogenous TCR α chain and β knocked out and expressing IgGs-GPC3-TCR, TCRs-GPC3-TCR, GMCSFs-GPC3-TCR, and GMCSFRas-GPC3-TCR, respectively. UTko is a T cell that has not been transduced with the recombinant TCR vector, but with the endogenous TCR α chain and β chain knockout. The positive rate of the recombinant TCR expression was detected at different time points of T cell activation (FIG. 4 and Table 4): the positive rate of the recombinant TCR on TCRT cells with endogenous TCR knocked out was significantly increased.

TABLE 4
Positive rate detection of the recombinant TCR

| | positive | positive | positive | positive | positive |
|---|---|---|---|---|---|
| | rate on | rate on | rate on | rate on | rate on |
| cell | D 8 | D 10 | D 13 | D 15 | D 17 |
| UT ko | 0.4% | 0.14% | 0.25% | 0.17% | 0.09% |
| TCRs-GPC3-TCRT2 | 35.2% | 17.8% | 16.1% | 11.3% | 3.25% |
| GMCSFs-GPC3- | 43.3% | 27.2% | 16.7% | 13.3% | 6.67% |
| TCRT2 | | | | | |
| IgGs-GPC3-TCRT2 | 65.1% | 49.2% | 38.9% | 38% | 21.6% |

### Example 9. GPC3-TCRT2 Kills Liver Cancer Cells In Vitro

Referring to Example 5. Target cells: PLC/PRF/5 cells (ATCC, USA); effector cells: UTko, IgGs-GPC3-TCRT2, TCRs-GPC3-TCRT2, GMCSFs-GPC3-TCRT2. The results show that compared to UTko, TCRT cells constructed with different signal peptides can significantly kill target cells after co-incubation for 24 or 48 hours (FIG. 5).

### Example 10. GPC3-TCRT Inhibits Subcutaneous Large-Load Transplanted Liver Tumors in NPG Mice

3×10⁶ PLC/PRF/5 was inoculated subcutaneously into the right axilla of female NPG mice (day D0). The average tumor volume on day D13 was about 250 mm³. The mice were divided into 3 groups, 5 mice in each group, and injected respectively into the tail vein: 3×106 UT, IgGs-GPC3-TCRT1, IgGs-GPC3-TCRT2 cells/mouse. The tumor volume was measured every 3-4 days, the transplanted tumor volume and body weight changes of the mice were record. The tumor volume calculation formula is: (length×width²)/2. Compared with the UT group, the tumor inhibition rates on D31 were: approximately 25.63% for IgGs-GPC3-TCRT1, and approximately 52.03% for IgGs-GPC3-TCRT2 (FIG. 6).

### Example 11. GPC3-TCRT2 Inhibits Subcutaneous Liver Transplanted Tumors in NOD/SCID Mice

Referring to Example 10. 3×10⁶ PLC/PRF/5 was inoculated subcutaneously into the right axilla of female NOD/SCID mice (Vital River) (day D0). The average tumor volume on day D12 was about 150 mm³. The mice were divided into 4 groups, with 5 mice in each group. Cyclophosphamide (100 mg/kg) was injected intraperitoneally; 24 hours later, 5×10⁶ UT and IgGs-GPC3-TCRT2 cells/mouse were injected into the tail vein respectively. After treatment, the mice were euthanized. Compared with UT, the tumor inhibition rate of the IgGs-GPC3-TCRT2 group on day D36 was approximately 85.54% (FIG. 7).

### Example 12. GPC3-TCRT2 Inhibits Large-Load Subcutaneous Liver Transplanted Tumors in NOD/SCID Mice

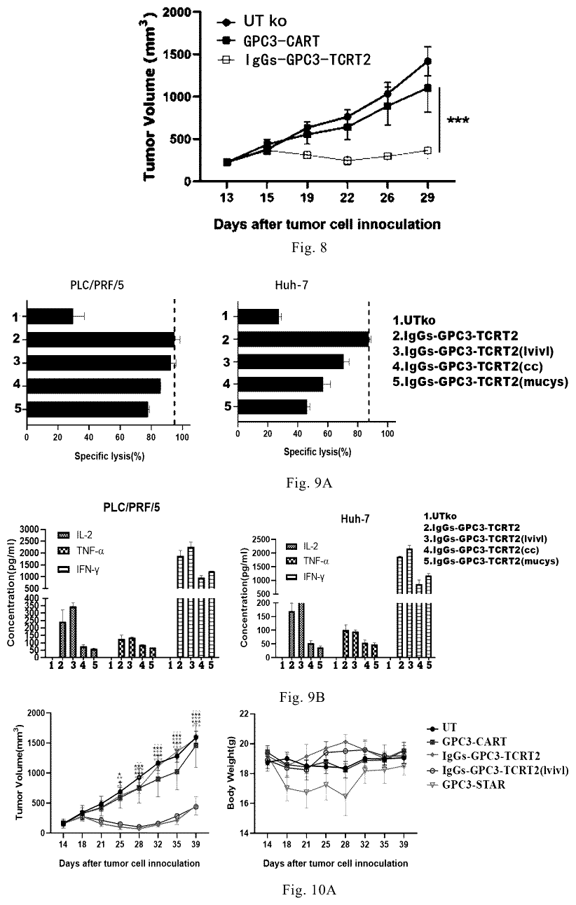

GPC3-CART expressing GPC3-CAR (SEQ ID NO: 52) was prepared according to conventional methods. Referring to Example 10. 3×10⁶ PLC/PRF/5 was inoculated subcutaneously into the right axilla of female NOD/SCID mice (day D0). The average tumor volume on day D12 was about 230 mm³. The mice were divided into 2 groups, with 5 mice in each group. Cyclophosphamide (100 mg/kg) was injected intraperitoneally; 24 hours later, 5×10⁶ UT, GPC3-CART, and IgGs-GPC3-TCRT2 cells/mouse were injected into the tail vein. Compared with UTko, the tumor inhibition rates on D29 were: approximately 22.18% for GPC3-CART and approximately 74.25% for IgGs-GPC3-TCRT2 (P<0.001) (FIG. 8).

### Example 13. Preparation of GPC3-TCRT Cells Comprising Recombinant TCRs with Modified TCR Constant Regions

Referring to Example 7. Recombinant TCR that targets the tumor antigen GPC3 and comprises an antibody signal peptide, and a synonymous mutation/cysteine modification/hydrophobic amino acid substitution in the TCR constant region:
IgGs-GPC3-TCR (lvivl) fragment: comprises in sequence IgGsH1, GPC3 antibody VH, TRAC nucleic acid fragment 2 (SEQ ID NO: 26), P2A, IgGsL1, GPC3 antibody VL, and TRBC nucleic acid fragment 1 (SEQ ID NO: 32);
IgGs-GPC3-TCR (cc) fragment: comprises in sequence IgGsH1, GPC3 antibody VH, TRAC nucleic acid fragment 3 (SEQ ID NO: 27), P2A, IgGsL1, GPC3 antibody VL, and TRBC nucleic acid fragment 2 (SEQ ID NO: 33);
IgGs-GPC3-TCR (mucys) fragment: comprises in sequence IgGsH1, GPC3 antibody VH, TRAC nucleic acid fragment 4 (SEQ ID NO: 28), P2A, IgGsL1, GPC3 antibody VL, and TRBC nucleic acid fragment 2 (SEQ ID NO: 33);
The above recombinant TCR fragments were constructed in lentiviral vectors. Among them, IgGsH1 (SEQ ID NO: 15), IgGsL1 (SEQ ID NO: 10), GPC3 antibody VH (SEQ ID NO: 38), GPC3 antibody VL (SEQ ID NO: 39), and P2A (SEQ ID NO: 46).

Referring to Example 8. IgGs-GPC3-TCRT2 (lvivl), IgGs-GPC3-TCRT2 (cc), IgGs-GPC3-TCRT2 (mucys), and GMCSFRas-GPC3-TCRT2 cells with endogenous TCR α chain and β chain knocked out and expressing IgGs-GPC3-TCR (lvivl), IgGs-GPC3-TCR (cc), IgGs-GPC3-TCR (mucys), and GMCSFRas-GPC3-TCR, respectively, were constructed. The positive rate of the recombinant TCR expression and the double positive rate of the recombinant TCR/CD3 were detected at different time points of T cell activation (Table 5).

TABLE 5
Detection of positive rate of the recombinant TCR
and double positive rate of the recombinant TCR/CD3

| | recombinant TCR | recombinant TCR/ |
|---|---|---|
| | (positive rate | CD3 (positive rate |
| cell | on D 9) | on D 9) |
| UT | 0.039% | 0.039% |
| IgGs-GPC3-TCRT2 | 58.4% | 38.2% |
| IgGs-GPC3-TCRT2(lvivl) | 63.1% | 53.5% |
| IgGs-GPC3-TCRT2(cc) | 78.1% | 78.0% |
| IgGs-GPC3-TCRT2(mucys) | 76.6% | 76.5% |
| GMCSFRas-GPC3-TCRT2 | 14.9% | 9.85% |

### Example 14. GPC3-TCRT Comprising the Recombinant TCR Comprising an Antibody Signal Peptide, and a Synonymous Mutation/Cysteine Modification/Hydrophobic Amino Acid Substitution in the TCR Constant Region Kills Liver Cancer Cells In Vitro

Referring to Example 5. Target cells: PLC/PRF/5 cells (ATCC, USA), Huh7 cells (Cell Collection Center, Chinese Academy of Sciences); effector cells: UTko, IgGs-GPC3-TCRT2, IgGs-GPC3-TCRT2 (lvivl), IgGs-GPC3-TCRT2 (cc), and IgGs-GPC3-TCRT2 (mucys) cells. The effector-to-target ratio was 1:1 and the cells were co-incubated for a total of 24 hours. Compared with UTko, the GPC3-TCRT effector cells have obvious killing effect on target cells (FIG. 9A) and can secrete higher levels of cytokines (FIG. 9B).

### Example 15. GPC3-TCRT Inhibits Subcutaneously Transplanted Liver Tumors in NOD/SCID Mice

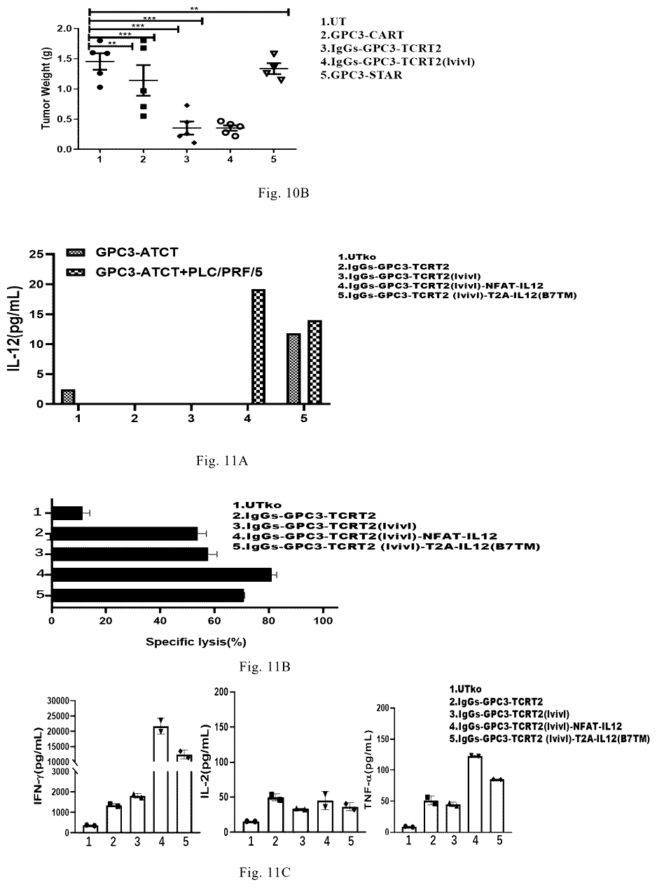

Referring to Example 11. 3.5×10⁶ PLC/PRF/5 was inoculated subcutaneously into the right axilla of female NOD/SCID mice (day D0). The average tumor volume on day D14 was about 159 mm³. The mice were divided into 7 groups, 5 mice in each group, and intraperitoneally injected with cyclophosphamide (100 mg/kg); 24 hours later, 5×10⁶ UT cells, GPC3-CART cells, IgGs-GPC3-TCRT2, IgGs-GPC3-TCRT2(lvivl), and GPC3-STAR-T cells/mouse were injected into the tail vein. The results are shown in FIG. 10. GPC3-STAR-T cells targeting GPC3 were prepared with reference to patent CN110818802A. After treatment, mice were euthanized. Compared with the UT group, the tumor inhibition rates of each group based on tumor weight on D39 were: 21.57% for the GPC3-CAR group, 75.69% for the IgGs-GPC3-TCRT2 group, 75.69% for the IgGs-GPC3-TCRT2 (lvivl) group, and 8.14% for the control TCR-T group (T cells expressing the polypeptide as shown in SEQ ID NO: 58) (FIG. 10).

### Example 16. Preparation of TCRT Cells Expressing Cytokines

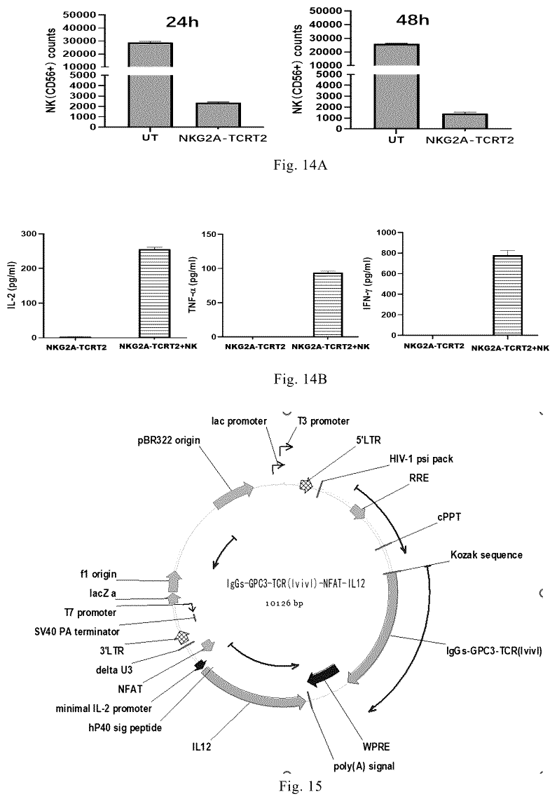

TCRT cells with the endogenous TCR α chain and β chain knocked out and expressing IL12 were constructed. As an example, the following cells were constructed:
(1) IgGs-GPC3-TCRT2 (lvivl)-NFAT-IL12 cells, whose construction vector is shown in FIG. 15; the cells express the IgGs-GPC3-TCR(lvivl) fragment and IL12 (SEQ ID NO: 35), wherein the expression of the IL12 is regulated by NFAT;
(2) IgGs-GPC3-TCRT2(lvivl)-T2A-IL12(B7TM) cells: expressing the polypeptide comprising in sequence IgGs-GPC3-TCR(lvivl) fragment, T2A (SEQ ID NO: 48), and IL12(B7TM) (SEQ ID NO:36);
Among them, the IgGs-GPC3-TCR (lvivl) fragment is as shown in Example 13. After co-incubation with PLC/PRF/5 cells, the expression of IL12 is shown in FIG. 11A.

### Example 17. TCRT-IL12 Expressing Cytokines Kills Liver Cancer Cells In Vitro

Referring to Example 5. Target cells: PLC/PRF/5 cells (ATCC, USA), Huh7 cells (Cell Collection Center, Chinese Academy of Sciences); effector cells: UTko, IgGs-GPC3-TCRT2, IgGs-GPC3-TCRT2 (lvivl), IgGs-GPC3-TCRT2(lvivl)-NFAT-IL12, and IgGs-GPC3-TCRT2(lvivl)-T2A-IL12(B7TM) cells. The effector-to-target ratio was 1:1 and the cells were co-incubated for a total of 24 hours. The results show that TCRT cells expressing IL12 significantly kill target cells (FIG. 11B) and can secrete higher levels of cytokines (FIG. 11C).

### Example 18. Preparation of GPRC5D-TCRT and BCMA-TCRT Cells

Referring to Example 7. Recombinant TCRs targeting the tumor antigen GPRC5D or BCMA and comprising different signal peptides:

IgGs-GPRC5D-TCR fragment: comprising in sequence IgGsH1, GPRC5D antibody VH, TRAC nucleic acid fragment 1, P2A, IgGsL1, GPRC5D antibody VL, and TRBC nucleic acid fragment 1;
IgGs-GPRC5D-TCR (mucys) fragment: comprising in sequence IgGsH1, GPRC5D antibody VH, TRAC nucleic acid fragment 4, P2A, IgGsL1, GPRC5D antibody VL, and TRBC nucleic acid fragment 2;
IgGs-BCMA-TCR fragment: comprising in sequence IgGsH1, BCMA antibody VH, TRAC nucleic acid fragment 1, P2A, IgGsL1, BCMA antibody VL, and TRBC nucleic acid fragment 1;
IgGs-BCMA-TCR (mucys) fragment: comprising in sequence IgGsH1, BCMA antibody VH, TRAC nucleic acid fragment 4, P2A, IgGsL1, BCMA antibody VL, and TRBC nucleic acid fragment 2;
The above recombinant TCR fragments were constructed respectively in lentiviral vectors. Among them, IgGsH1 (SEQ ID NO: 15), IgGsL1 (SEQ ID NO: 10), GPRC5D antibody VH (SEQ ID NO: 40), GPRC5D antibody VL (SEQ ID NO: 41), BCMA antibody VH (SEQ ID NO: 42), BCMA antibody VL (SEQ ID NO: 43), TRAC nucleic acid fragment 1 (SEQ ID NO: 25), TRAC nucleic acid fragment 4 (SEQ ID NO: 28), TRBC nucleic acid fragment 1 (SEQ ID NO: 32), TRBC nucleic acid fragment 2 (SEQ ID NO: 33), and P2A (SEQ ID NO: 46).

Referring to the foregoing examples. The following TCRT cells were constructed: IgGs-GPRC5D-TCRT1, IgGs-GPRC5D-TCRT1(mucys), IgGs-BCMA-TCRT1, and IgGs-BCMA-TCRT1(mucys); and IgGs-GPRC5D-TCRT2, IgGs-GPRC5D-TCRT2(mucys), IgGs-BCMA-TCRT2, and IgGs-BCMA-TCRT2(mucys) with the endogenous TCR α chain and β chain knocked out. The positive rate of the recombinant TCR expression was then detected at different time points of T cell activation (Table 6).

TABLE 6
Positive rate detection of the recombinant TCR

| cell | D 9 | D 13 |
|---|---|---|
| UTko | 4.83% | 1.44% |
| IgGs-GPRC5D-TCRT2 | 70.1% | 58.8% |
| IgGs-GPRC5D-TCRT1(mucys) | 85.4% | 70.4% |
| IgGs-GPRC5D-TCRT2(mucys) | 89.9% | 88.7% |
| IgGs-BCMA-TCRT1 | 33.6% | 31.9% |
| IgGs-BCMA-TCRT2 | 77.5% | 67.4% |
| IgGs-BCMA-TCRT1(mucys) | 48.5% | 49.9% |
| IgGs-BCMA-TCRT2(mucys) | 51.5% | 54.7% |

### Example 19. GPRC5D-TCRT and BCMA-TCRT Kill Multiple Myeloma Cells In Vitro

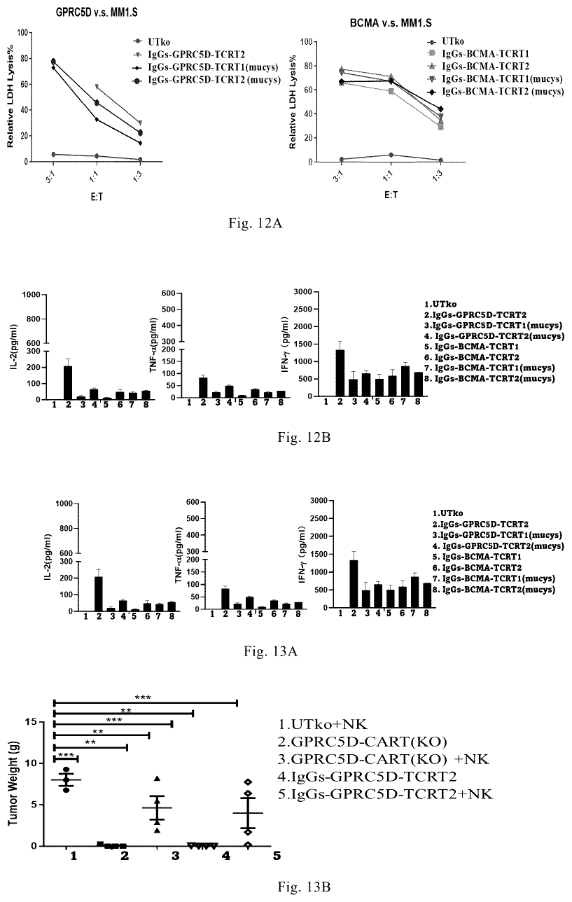

After the effector cells and target cells MM1.S (15000 cells/well) were co-incubated for 18 hours at a ratio of 3:1/1:1/1:3, LDH was used to detect cytotoxicity. The results show: compared to UTko, IgGs-GPRC5D-TCRT2, IgGs-GPRC5D-TCRT1(mucys), IgGs-GPRC5D-TCRT2(mucys), IgGs-BCMA-TCRT1, IgGs-BCMA-TCRT1(mucys), IgGs-BCMA-TCRT2 and IgGs-BCMA-TCRT2(mucys) all significantly kill target cells (FIG. 12A), and can secrete higher levels of cytokines (using Biolegend's detection kit to perform CBA detection of the supernatant, FIG. 12B).

### Example 20. GPRC5D-TCRT Inhibits Subcutaneous Large Load Multiple Myeloma in NPG Mice in the Presence of NK Cells

GPRC5D-CART (KO) cells (T cells with endogenous TCR and B2M knocked out and expressing GPRC5D-CAR (SEQ ID NO: 53) were prepared according to conventional methods). The gRNA-TRAC and gRNA-B2M used were as shown in SEQ ID NO: 49 and 51, respectively.

Referring to the foregoing examples. 3×10⁶ MM.1S cells were inoculated subcutaneously into the right axilla of female NPG mice (day D0). The average tumor volume on D16 was approximately 238 mm³, and the mice were divided into 5 groups, with 4 mice in each group. Starting from day D17, NK was injected a total of 5 times (2×10\hat{ }6/time). On day D18, the tumor volume was approximately 400 mm³, and 2×10⁶ GPRC5D-CART (KO) and IgGs-GPRC5D-TCRT2 cells/mouse were injected into the tail vein, respectively. After treatment, mice were euthanized. Compared to the UTko+NK group, the inhibition rates based on tumor weight on D45 were: 99.22% for the GPRC5D-CART (KO) group, 42.3% for the GPRC5D-CART (KO)+NK group, 99.84% for the IgGs-GPRC5D-TCRT2 group, and 50.14% for the IgGs-GPRC5D-TCRT2+NK group (FIG. 13).

### Example 21. Preparation of NKG2A-TCRT2 Cells with Endogenous TCR and B2M Knocked Out

Exemplary, the recombinant TCR targeting NKG2A:

NKG2A-TCR fragment: comprising in sequence IgGsH1 (SEQ ID NO:15), NKG2A antibody VH (SEQ ID NO:44), TRAC nucleic acid fragment 1 (SEQ ID NO:25), P2A (SEQ ID NO:46), IgGsL1 (SEQ ID NO: 10), NKG2A antibody VL (SEQ ID NO: 45), and TRBC nucleic acid fragment 1 (SEQ ID NO: 32).

NKG2A-TCRT2 cells with endogenous TCR and B2M knocked out and expressing NKG2A-TCR fragment were prepared with reference to the foregoing examples. The gRNA-TRAC, gRNA-TRBC, and gRNA-B2M used were as shown in SEQ ID NO: 49, 50, and 51, respectively.

### Example 22.NKG2A-TCRT2 Kills NK Cells In Vitro

Referring to the foregoing examples. Target cells: NK (30000 cells/well); effector cells: UT, IgGs-NKG2A-TCRT2. The effector-to-target ratio was 1:1, and co-incubated for 24 or 48 hours. The results show that compared to UT, NKG2A-TCRT2 significantly kills NK (FIG. 14A). Moreover, compared with NKG2A-TCRT2 alone, higher concentration levels of cytokines can be detected in the supernatant of the NKG2A-TCRT group co-incubated with NK cells (FIG. 14B).

The examples described herein comprise this example as any single example or in combination with any other examples or portions thereof. In addition, it should be understood that after reading the above content of the present application, those skilled in the art can make various changes or modifications to the present application, and the equivalent forms of these changes or modifications also fall within the scope of the appended claims of the present application.

Information of the sequences

| | | |
|---|---|---|
| 1 | amino acid | MAMLLGASVLILWLQPDWVNSQQKNDD |
| | sequence of | |
| | TRAVs | |
| 2 | nucleic acid | ATGGCCATGCTCCTGGGGGCATCAGTGCTGATTCTGTGGCTTC |
| | sequence of | AGCCAGACTGGGTAAACAGTCAACAGAAGAATGATGAC |
| | TRAVs | |
| 3 | amino acid | MGTSLLCWMALCLLGADHADT |
| | sequence of | |
| | TRBVs | |
| 4 | nucleic acid | ATGGGCACCAGCCTCCTCTGCTGGATGGCCCTGTGTCTCCTG |
| | sequence of | GGGGCAGATCACGCAGATACT |
| | TRBVs | |
| 5 | amino acid | MWLQSLLLLGTVACSIS |
| | sequence of | |
| | GMCSFs | |
| 6 | nucleic acid | ATGTGGCTGCAGAGCCTGCTGCTCTTGGGCACTGTGGCCTGC |
| | sequence of | AGCATCTCT |
| | GMCSFs | |
| 7 | amino acid | MLLLVTSLLLCELPHPAFLLIP |
| | sequence of | |
| | GMCSFRas | |
| 8 | nucleic acid | ATGCTTCTCCTGGTGACAAGCCTTCTGCTCTGTGAGTTACCAC |
| | sequence of | ACCCAGCATTCCTCCTGATCCCA |
| | GMCSFRas | |
| 9 | amino acid | MDMRVPAQLLGLLLLWLSGARC |
| | sequence of | |
| | IgGsL1 | |
| 10 | nucleic acid | ATGGACATGAGGGTCCCTGCTCAGCTCCTGGGGCTCCTGCTG |
| | sequence of | CTCTGGCTCTCAGGTGCCAGATGT |
| | IgGsL1 | |
| 11 | amino acid | MKYLLPTAAAGLLLLAAQPAMA |
| | sequence of | |
| | IgGsL2 | |
| 12 | amino acid | MVLQTQVFISLLLWIS |
| | sequence of | |
| | IgGsL3 | |
| 13 | amino acid | MAWTPLFLFLLTCCPG |
| | sequence of | |
| | IgGsL4 | |
| 14 | amino acid | MEFGLSWVFLVALFRGVQC |
| | sequence of | |
| | IgGsH1 | |
| 15 | nucleic acid | ATGGAGTTTGGGCTGAGCTGGGTTTTCCTCGTTGCTCTTTTTA |
| | sequence of | GAGGTGTCCAGTGT |
| | IgGsH1 | |
| 16 | amino acid | MDWTWRFLFVVAAATGVQS |
| | sequence of | |
| | IgGsH2 | |
| 17 | amino acid | MDLLHKNMKHLWFFLLLVAAPRWVLS |
| | sequence of | |
| | IgGsH3 | |
| 18 | amino acid | MDWTWRILFLVAAATG |
| | sequence of | |
| | IgGsH4 | |
| 19 | amino acid | IQNPDPAVYQLRDSKSSDKSVCLFTDFDSQTNVSQSKDSDVY |
| | sequence of | ITDKTVLDMRSMDFKSNSAVAWSNKSDFACANAFNNSIIPED |
| | TRAC | TFFPSPESSCDVKLVEKSFETDTNLNFQNLSVIGFRILLLKVA |
| | | GFNLLMTLRLWSS |
| 20 | nucleic acid | ATCCAGAACCCTGACCCTGCCGTGTACCAGCTGAGAGACTCT |
| | sequence of | AAATCCAGTGACAAGTCTGTCTGCCTATTCACCGATTTTGATT |
| | TRAC (wild | CTCAAACAAATGTGTCACAAAGTAAGGATTCTGATGTGTATAT |
| | type) | CACAGACAAAACTGTGCTAGACATGAGGTCTATGGACTTCAA |
| | | GAGCAACAGTGCTGTGGCCTGGAGCAACAAATCTGACTTTG |
| | | CATGTGCAAACGCCTTCAACAACAGCATTATTCCAGAAGACA |
| | | CCTTCTTCCCCAGCCCAGAAAGTTCCTGTGATGTCAAGCTGG |
| | | TCGAGAAAAGCTTTGAAACAGATACGAACCTAAACTTTCAA |
| | | AACCTGTCAGTGATTGGGTTCCGAATCCTCCTCCTGAAAGTG |
| | | GCCGGGTTTAATCTGCTCATGACGCTGCGGCTGTGGTCCAGC |
| 21 | amino acid | EDLNKVFPPEVAVFEPSEAEISHTQKATLVCLATGFFPDHVEL |
| | sequence of | SWWVNGKEVHSGVSTDPQPLKEQPALNDSRYCLSSRLRVSA |
| | TRBC | TFWQNPRNHFRCQVQFYGLSENDEWTQDRAKPVTQIVSAEA |
| | | WGRADCGFTSVSYQQGVLSATILYEILLGKATLYAVLVSAL |
| | | VLMAMVKRKDF |
| 22 | nucleic acid | GAGGACCTGAACAAGGTGTTCCCACCCGAGGTCGCTGTGTT |
| | sequence of | TGAGCCATCAGAAGCAGAGATCTCCCACACCCAAAAGGCCA |
| | TRBC (wild | CACTGGTGTGCCTGGCCACAGGCTTCTTCCCCGACCACGTGG |
| | type) | AGCTGAGCTGGTGGGTGAATGGGAAGGAGGTGCACAGTGGG |
| | | GTCAGCACGGACCCGCAGCCCCTCAAGGAGCAGCCCGCCCT |
| | | CAATGACTCCAGATACTGCCTGAGCAGCCGCCTGAGGGTCTC |
| | | GGCCACCTTCTGGCAGAACCCCCGCAACCACTTCCGCTGTCA |
| | | AGTCCAGTTCTACGGGCTCTCGGAGAATGACGAGTGGACCC |
| | | AGGATAGGGCCAAACCCGTCACCCAGATCGTCAGCGCCGAG |
| | | GCCTGGGGTAGAGCAGACTGTGGCTTTACCTCGGTGTCCTAC |
| | | CAGCAAGGGGTCCTGTCTGCCACCATCCTCTATGAGATCCTG |
| | | CTAGGGAAGGCCACCCTGTATGCTGTGCTGGTCAGCGCCCTT |
| | | GTGTTGATGGCCATGGTCAAGAGAAAGGATTTC |
| 23 | amino acid | DKQLDADVSPKPTIFLPSIAETKLQKAGTYLCLLEKFFPDVIKI |
| | sequence of | HWQEKKSNTILGSQEGNTMKTNDTYMKFSWLTVPEKSLDK |
| | TRGC | EHRCIVRHENNKNGVDQEIIFPPIKTDVITMDPKDNCSKDAN |
| | | DTLLLQLTNTSAYYMYLLLLLKSVVYFAIITCCLLRRTAFCC |
| | | NGEKS |
| 24 | amino acid | SQPHTKPSVFVMKNGTNVACLVKEFYPKDIRINLVSSKKITEF |
| | sequence of | DPAIVISPSGKYNAVKLGKYEDSNSVTCSVQHDNKTVHSTDF |
| | TRDC | EVKTDSTDHVKPKETENTKQPSKSCHKPKAIVHTEKVNMMS |
| | | LTVLGLRMLFAKTVAVNFLLTAKLFFL |
| 25 | TRAC | ATCCAGAACCCTGACCCTGCCGTGTATCAGCTGAGGGATT |
| | nucleic acid | CCAAATCCAGTGACAAGTCTGTCTGCCTATTCACCGATTTT |
| | fragment 1 | GATTCTCAAACAAATGTGTCACAAAGTAAGGATTCTGATG |
| | | TGTATATCACAGACAAAACTGTGCTAGACATGAGGTCTAT |
| | | GGACTTCAAGAGCAACAGTGCTGTGGCCTGGAGCAACAA |
| | | ATCTGACTTTGCATGTGCAAACGCCTTCAACAACAGCATT |
| | | ATTCCAGAAGACACCTTCTTCCCCAGCCCAGAAAGTTCCT |
| | | GTGATGTCAAGCTGGTCGAGAAAAGCTTTGAAACAGATAC |
| | | GAACCTAAACTTTCAAAACCTGTCAGTGATTGGGTTCCGA |
| | | ATCCTCCTCCTGAAAGTGGCCGGGTTTAATCTGCTCATGA |
| | | CGCTGCGGCTGTGGTCCAGC |
| 26 | TRAC | ATCCAGAACCCTGACCCTGCCGTGTATCAGCTGAGGGATT |
| | nucleic acid | CCAAATCCAGTGACAAGTCTGTCTGCCTATTCACCGATTTT |
| | fragment 2 | GATTCTCAAACAAATGTGTCACAAAGTAAGGATTCTGATG |
| | | TGTATATCACAGACAAAACTGTGCTAGACATGAGGTCTAT |
| | | GGACTTCAAGAGCAACAGTGCTGTGGCCTGGAGCAACAA |
| | | ATCTGACTTTGCATGTGCAAACGCCTTCAACAACAGCATT |
| | | ATTCCAGAAGACACCTTCTTCCCCAGCCCAGAAAGTTCCT |
| | | GTGATGTCAAGCTGGTCGAGAAAAGCTTTGAAACAGATAC |
| | | GAACCTAAACTTTCAAAACCTGCTGGTGATCGTGCTGCGA |
| | | ATCCTCCTCCTGAAAGTGGCCGGGTTTAATCTGCTCATGA |
| | | CGCTGCGGCTGTGGTCCAGC |
| 27 | TRAC | ATCCAGAACCCTGACCCTGCCGTGTATCAGCTGAGGGATT |
| | nucleic acid | CCAAATCCAGTGACAAGTCTGTCTGCCTATTCACCGATTTT |
| | fragment 3 | GATTCTCAAACAAATGTGTCACAAAGTAAGGATTCTGATG |
| | | TGTATATCACAGACAAATGCGTGCTAGACATGAGGTCTAT |
| | | GGACTTCAAGAGCAACAGTGCTGTGGCCTGGAGCAACAA |
| | | ATCTGACTTTGCATGTGCAAACGCCTTCAACAACAGCATT |
| | | ATTCCAGAAGACACCTTCTTCCCCAGCCCAGAAAGTTCCT |
| | | GTGATGTCAAGCTGGTCGAGAAAAGCTTTGAAACAGATAC |
| | | GAACCTAAACTTTCAAAACCTGTCAGTGATTGGGTTCCGA |
| | | ATCCTCCTCCTGAAAGTGGCCGGGTTTAATCTGCTCATGA |
| | | CGCTGCGGCTGTGGTCCAGC |
| 28 | TRAC | ATCCAGAACCCTGACCCTGCCGTGTATCAGCTGAGGGATT |
| | nucleic acid | CCAAATCCAGTGACAAGTCTGTCTGCCTATTCACCGATTTT |
| | fragment 4 | GATTCTCAAACAAATGTGTCACAAAGTAAGGATTCTGATG |
| | | TGTATATCACAGACAAATGCGTGCTAGACATGAGGTCTAT |
| | | GGACTTCAAGAGCAACAGTGCTGTGGCCTGGAGCAACAA |
| | | ATCTGACTTTGCATGTGCAAACGCCTTCAACAACAGCATT |
| | | ATTCCAGAAGACACCTTCTTCCCCAGCCCAGAAAGTTCCT |
| | | GTGATGTCAAGCTGGTCGAGAAAAGCTTTGAAACAGATAC |
| | | GAACCTAAACTTTCAAAACCTGCTGGTGATCGTGCTGCGA |
| | | ATCCTCCTCCTGAAAGTGGCCGGGTTTAATCTGCTCATGA |
| | | CGCTGCGGCTGTGGTCCAGC |
| 29 | TRAC | ATCCAGAACCCTGACCCTGCCGTGTACCAGCTGAGAGACTCT |
| | nucleic acid | AAATCCAGTGACAAGTCTGTCTGCCTATTCACCGATTTTGATT |
| | fragment 5 | CTCAAACAAATGTGTCACAAAGTAAGGATTCTGATGTGTATAT |
| | | CACAGACAAAACTGTGCTAGACATGAGGTCTATGGACTTCAA |
| | | GAGCAACAGTGCTGTGGCCTGGAGCAACAAATCTGACTTTG |
| | | CATGTGCAAACGCCTTCAACAACAGCATTATTCCAGAAGACA |
| | | CCTTCTTCCCCAGCCCAGAAAGTTCCTGTGATGTCAAGCTGG |
| | | TCGAGAAAAGCTTTGAAACAGATACGAACCTAAACTTTCAA |
| | | AACCTGCTGGTGATCGTGCTGCGAATCCTCCTCCTGAAAGT |
| | | GGCCGGGTTTAATCTGCTCATGACGCTGCGGCTGTGGTCCAG |
| | | C |
| 30 | TRAC | ATCCAGAACCCTGACCCTGCCGTGTACCAGCTGAGAGACTCT |
| | nucleic acid | AAATCCAGTGACAAGTCTGTCTGCCTATTCACCGATTTTGATT |
| | fragment 6 | CTCAAACAAATGTGTCACAAAGTAAGGATTCTGATGTGTATAT |
| | | CACAGACAAATGCGTGCTAGACATGAGGTCTATGGACTTCAA |
| | | GAGCAACAGTGCTGTGGCCTGGAGCAACAAATCTGACTTTG |
| | | CATGTGCAAACGCCTTCAACAACAGCATTATTCCAGAAGACA |
| | | CCTTCTTCCCCAGCCCAGAAAGTTCCTGTGATGTCAAGCTGG |
| | | TCGAGAAAAGCTTTGAAACAGATACGAACCTAAACTTTCAA |
| | | AACCTGTCAGTGATTGGGTTCCGAATCCTCCTCCTGAAAGTG |
| | | GCCGGGTTTAATCTGCTCATGACGCTGCGGCTGTGGTCCAGC |
| 31 | TRAC | ATCCAGAACCCTGACCCTGCCGTGTACCAGCTGAGAGACTC |
| | nucleic acid | TAAATCCAGTGACAAGTCTGTCTGCCTATTCACCGATTTTG |
| | fragment 7 | ATTCTCAAACAAATGTGTCACAAAGTAAGGATTCTGATGT |
| | | GTATATCACAGACAAATGCGTGCTAGACATGAGGTCTATG |
| | | GACTTCAAGAGCAACAGTGCTGTGGCCTGGAGCAACAAA |
| | | TCTGACTTTGCATGTGCAAACGCCTTCAACAACAGCATTA |
| | | TTCCAGAAGACACCTTCTTCCCCAGCCCAGAAAGTTCCTG |
| | | TGATGTCAAGCTGGTCGAGAAAAGCTTTGAAACAGATACG |
| | | AACCTAAACTTTCAAAACCTGCTGGTGATCGTGCTGCGAA |
| | | TCCTCCTCCTGAAAGTGGCCGGGTTTAATCTGCTCATGAC |
| | | GCTGCGGCTGTGGTCCAGC |
| 32 | TRBC | GAGGACCTGAACAAGGTGTTCCCACCCGAGGTCGCTGTGTT |
| | nucleic acid | TGAGCCATCAGAAGCAGAGATCTCCCACACCCAAAAGGCCA |
| | fragment 1 | CACTGGTGTGCCTGGCCACAGGCTTCTTCCCCGACCACGTGG |
| | | AGCTGAGCTGGTGGGTGAATGGGAAGGAGGTGCACAGTGGG |
| | | GTCAGCACGGACCCGCAGCCCCTCAAGGAGCAGCCCGCCCT |
| | | CAATGACTCCAGATACTGCCTGAGCAGCCGCCTGAGGGTCTC |
| | | GGCCACCTTCTGGCAGAACCCCCGCAACCACTTCCGCTGTCA |
| | | AGTCCAGTTCTACGGGCTCTCGGAGAATGACGAGTGGACCC |
| | | AGGATAGGGCCAAACCCGTCACCCAGATTGTCTCCGCCGAA |
| | | GCCTGGGGTAGAGCAGACTGTGGCTTTACCTCGGTGTCCTAC |
| | | CAGCAAGGGGTCCTGTCTGCCACCATCCTCTATGAGATCCTG |
| | | CTAGGGAAGGCCACCCTGTATGCTGTGCTGGTCAGCGCCCTT |
| | | GTGTTGATGGCCATGGTCAAGAGAAAGGATTTC |
| 33 | TRBC | GAGGACCTGAACAAGGTGTTCCCACCCGAGGTCGCTGTGT |
| | nucleic acid | TTGAGCCATCAGAAGCAGAGATCTCCCACACCCAAAAGG |
| | fragment 2 | CCACACTGGTGTGCCTGGCCACAGGCTTCTTCCCCGACCA |
| | | CGTGGAGCTGAGCTGGTGGGTGAATGGGAAGGAGGTGCA |
| | | CAGTGGGGTCTGCACGGACCCGCAGCCCCTCAAGGAGCA |
| | | GCCCGCCCTCAATGACTCCAGATACTGCCTGAGCAGCCGC |
| | | CTGAGGGTCTCGGCCACCTTCTGGCAGAACCCCCGCAACC |
| | | ACTTCCGCTGTCAAGTCCAGTTCTACGGGCTCTCGGAGAA |
| | | TGACGAGTGGACCCAGGATAGGGCCAAACCCGTCACCCA |
| | | GATTGTCTCCGCCGAAGCCTGGGGTAGAGCAGACTGTGGC |
| | | TTTACCTCGGTGTCCTACCAGCAAGGGGTCCTGTCTGCCA |
| | | CCATCCTCTATGAGATCCTGCTAGGGAAGGCCACCCTGTA |
| | | TGCTGTGCTGGTCAGCGCCCTTGTGTTGATGGCCATGGTC |
| | | AAGAGAAAGGATTTC |
| 34 | TRBC | GAGGACCTGAACAAGGTGTTCCCACCCGAGGTCGCTGTGTT |
| | nucleic acid | TGAGCCATCAGAAGCAGAGATCTCCCACACCCAAAAGGCCA |
| | fragment 3 | CACTGGTGTGCCTGGCCACAGGCTTCTTCCCCGACCACGTGG |
| | | AGCTGAGCTGGTGGGTGAATGGGAAGGAGGTGCACAGTGGG |
| | | GTCTGCACGGACCCGCAGCCCCTCAAGGAGCAGCCCGCCCT |
| | | CAATGACTCCAGATACTGCCTGAGCAGCCGCCTGAGGGTCTC |
| | | GGCCACCTTCTGGCAGAACCCCCGCAACCACTTCCGCTGTCA |
| | | AGTCCAGTTCTACGGGCTCTCGGAGAATGACGAGTGGACCC |
| | | AGGATAGGGCCAAACCCGTCACCCAGATCGTCAGCGCCGAG |
| | | GCCTGGGGTAGAGCAGACTGTGGCTTTACCTCGGTGTCCTAC |
| | | CAGCAAGGGGTCCTGTCTGCCACCATCCTCTATGAGATCCTG |
| | | CTAGGGAAGGCCACCCTGTATGCTGTGCTGGTCAGCGCCCTT |
| | | GTGTTGATGGCCATGGTCAAGAGAAAGGATTTC |
| 35 | Nucleic acid | ATGTGTCACCAGCAGTTGGTCATCTCTTGGTTTTCCCTGGTTT |
| | sequence of | TTCTGGCATCTCCCCTCGTGGCCATATGGGAACTGAAGAAAG |
| | IL12 | ATGTTTATGTCGTAGAATTGGATTGGTATCCGGATGCCCCTGG |
| | | AGAAATGGTGGTCCTCACCTGTGACACCCCTGAAGAAGATG |
| | | GTATCACCTGGACCTTGGACCAGAGCAGTGAGGTCTTAGGCT |
| | | CTGGCAAAACCCTGACCATCCAAGTCAAAGAGTTTGGAGAT |
| | | GCTGGCCAGTACACCTGTCACAAAGGAGGCGAGGTTCTAAG |
| | | CCATTCGCTCCTGCTGCTTCACAAAAAGGAAGATGGAATTTG |
| | | GTCCACTGATATTTTAAAGGACCAGAAAGAACCCAAAAATAA |
| | | GACCTTTCTAAGATGCGAGGCCAAGAATTATTCTGGACGTTT |
| | | CACCTGCTGGTGGCTGACGACAATCAGTACTGATTTGACATT |
| | | CAGTGTCAAAAGCAGCAGAGGCTCTTCTGACCCCCAAGGGG |
| | | TGACGTGCGGAGCTGCTACACTCTCTGCAGAGAGAGTCAGA |
| | | GGGGACAACAAGGAGTATGAGTACTCAGTGGAGTGCCAGGA |
| | | GGACAGTGCCTGCCCAGCTGCTGAGGAGAGTCTGCCCATTG |
| | | AGGTCATGGTGGATGCCGTTCACAAGCTCAAGTATGAAAACT |
| | | ACACCAGCAGCTTCTTCATCAGGGACATCATCAAACCTGACC |
| | | CACCCAAGAACTTGCAGCTGAAGCCATTAAAGAATTCTCGGC |
| | | AGGTGGAGGTCAGCTGGGAGTACCCTGACACCTGGAGTACT |
| | | CCACATTCCTACTTCTCCCTGACATTCTGCGTTCAGGTCCAGG |
| | | GCAAGAGCAAGAGAGAAAAGAAAGATAGAGTCTTCACGGA |
| | | CAAGACCTCAGCCACGGTCATCTGCCGCAAAAATGCCAGCAT |
| | | TAGCGTGCGGGCCCAGGACCGCTACTATAGCTCATCTTGGAG |
| | | CGAATGGGCATCTGTGCCCTGCAGTGGTGGCGGTGGCTCGGG |
| | | CGGTGGTGGGTCGGGTGGCGGCGGATCTAGAAACCTCCCCG |
| | | TGGCCACTCCAGACCCAGGAATGTTCCCATGCCTTCACCACT |
| | | CCCAAAACCTGCTGAGGGCCGTCAGCAACATGCTCCAGAAG |
| | | GCCAGACAAACTCTAGAATTTTACCCTTGCACTTCTGAAGAG |
| | | ATTGATCATGAAGATATCACAAAAGATAAAACCAGCACAGTG |
| | | GAGGCCTGTTTACCATTGGAATTAACCAAGAATGAGAGTTGC |
| | | CTAAATTCCAGAGAGACCTCTTTCATAACTAATGGGAGTTGCC |
| | | TGGCCTCCAGAAAGACCTCTTTTATGATGGCCCTGTGCCTTAG |
| | | TAGTATTTATGAAGACTTGAAGATGTACCAGGTGGAGTTCAA |
| | | GACCATGAATGCAAAGCTTCTGATGGATCCTAAGAGGCAGAT |
| | | CTTTCTAGATCAAAACATGCTGGCAGTTATTGATGAGCTGATG |
| | | CAGGCCCTGAATTTCAACAGTGAGACTGTGCCACAAAAATCC |
| | | TCCCTTGAAGAACCGGATTTTTATAAAACTAAAATCAAGCTCT |
| | | GCATACTTCTTCATGCTTTCAGAATTCGGGCAGTGACTATTGA |
| | | TAGAGTGATGAGCTATCTGAATGCTTCC |
| 36 | nucleic acid | ATGTGTCACCAGCAGTTGGTCATCTCTTGGTTTTCCCTGGTTT |
| | sequence of | TTCTGGCATCTCCCCTCGTGGCCATATGGGAACTGAAGAAAG |
| | IL12 (B7TM) | ATGTTTATGTCGTAGAATTGGATTGGTATCCGGATGCCCCTGG |
| | | AGAAATGGTGGTCCTCACCTGTGACACCCCTGAAGAAGATG |
| | | GTATCACCTGGACCTTGGACCAGAGCAGTGAGGTCTTAGGCT |
| | | CTGGCAAAACCCTGACCATCCAAGTCAAAGAGTTTGGAGAT |
| | | GCTGGCCAGTACACCTGTCACAAAGGAGGCGAGGTTCTAAG |
| | | CCATTCGCTCCTGCTGCTTCACAAAAAGGAAGATGGAATTTG |
| | | GTCCACTGATATTTTAAAGGACCAGAAAGAACCCAAAAATAA |
| | | GACCTTTCTAAGATGCGAGGCCAAGAATTATTCTGGACGTTT |
| | | CACCTGCTGGTGGCTGACGACAATCAGTACTGATTTGACATT |
| | | CAGTGTCAAAAGCAGCAGAGGCTCTTCTGACCCCCAAGGGG |
| | | TGACGTGCGGAGCTGCTACACTCTCTGCAGAGAGAGTCAGA |
| | | GGGGACAACAAGGAGTATGAGTACTCAGTGGAGTGCCAGGA |
| | | GGACAGTGCCTGCCCAGCTGCTGAGGAGAGTCTGCCCATTG |
| | | AGGTCATGGTGGATGCCGTTCACAAGCTCAAGTATGAAAACT |
| | | ACACCAGCAGCTTCTTCATCAGGGACATCATCAAACCTGACC |
| | | CACCCAAGAACTTGCAGCTGAAGCCATTAAAGAATTCTCGGC |
| | | AGGTGGAGGTCAGCTGGGAGTACCCTGACACCTGGAGTACT |
| | | CCACATTCCTACTTCTCCCTGACATTCTGCGTTCAGGTCCAGG |
| | | GCAAGAGCAAGAGAGAAAAGAAAGATAGAGTCTTCACGGA |
| | | CAAGACCTCAGCCACGGTCATCTGCCGCAAAAATGCCAGCAT |
| | | TAGCGTGCGGGCCCAGGACCGCTACTATAGCTCATCTTGGAG |
| | | CGAATGGGCATCTGTGCCCTGCAGTGGTGGCGGTGGCTCGGG |
| | | CGGTGGTGGGTCGGGTGGCGGCGGATCTAGAAACCTCCCCG |
| | | TGGCCACTCCAGACCCAGGAATGTTCCCATGCCTTCACCACT |
| | | CCCAAAACCTGCTGAGGGCCGTCAGCAACATGCTCCAGAAG |
| | | GCCAGACAAACTCTAGAATTTTACCCTTGCACTTCTGAAGAG |
| | | ATTGATCATGAAGATATCACAAAAGATAAAACCAGCACAGTG |
| | | GAGGCCTGTTTACCATTGGAATTAACCAAGAATGAGAGTTGC |
| | | CTAAATTCCAGAGAGACCTCTTTCATAACTAATGGGAGTTGCC |
| | | TGGCCTCCAGAAAGACCTCTTTTATGATGGCCCTGTGCCTTAG |
| | | TAGTATTTATGAAGACTTGAAGATGTACCAGGTGGAGTTCAA |
| | | GACCATGAATGCAAAGCTTCTGATGGATCCTAAGAGGCAGAT |
| | | CTTTCTAGATCAAAACATGCTGGCAGTTATTGATGAGCTGATG |
| | | CAGGCCCTGAATTTCAACAGTGAGACTGTGCCACAAAAATCC |
| | | TCCCTTGAAGAACCGGATTTTTATAAAACTAAAATCAAGCTCT |
| | | GCATACTTCTTCATGCTTTCAGAATTCGGGCAGTGACTATTGA |
| | | TAGAGTGATGAGCTATCTGAATGCTTCCGATAACCTGCTCCCA |
| | | TCCTGGGCCATTACCTTAATCTCAGTAAATGGAATTTTTGTGAT |
| | | ATGCTGCCTGACCTACTGCTTTGCCCCAAGATGCAGAGAGAG |
| | | AAGGAGGAATGAGAGATTGAGAAGGGAAAGTGTACGCCCTG |
| | | TA |
| 37 | nucleic acid | GGAGGAAAAACTGTTTCATACAGAAGGCGTGGAGGAAAAAC |
| | sequence of | TGTTTCATACAGAAGGCGTGGAGGAAAAACTGTTTCATACAG |
| | NFAT6 | AAGGCGTCAATTGTCCTCGACGGAGGAAAAACTGTTTCATAC |
| | binding | AGAAGGCGTGGAGGAAAAACTGTTTCATACAGAAGGCGTGG |
| | motif | AGGAAAAACTGTTTCATACAGAAGGCGT |
| 38 | nucleic acid | GAGGTGCAGCTGGTGCAGAGCGGCGCCGAGGTGAAGAAGC |
| | sequence of | CCGGCGCCAGCGTGAAGGTGAGCTGCAAGGCCAGCGGCTAC |
| | GPC3 | ACCTTCAGCGACTACGAGATGCACTGGGTGCGGCAGGCCCC |
| | antibody VH | CGGCCAGGGCCTGGAGTGGATGGGCGCCATCCACCCCGGCA |
| | | GCGGCGACACCGCCTACAACCAGCGGTTCAAGGGCCGGGTG |
| | | ACCATCACCGCCGACAAGAGCACCAGCACCGCCTACATGGA |
| | | GCTGAGCAGCCTGCGGAGCGAGGACACCGCCGTGTACTACT |
| | | GCGCCCGGTTCTACAGCTACGCCTACTGGGGCCAGGGCACCC |
| | | TGGTGACCGTGAGCGCC |
| 39 | nucleic acid | GACATCGTGATGACCCAGACCCCCCTGAGCCTGCCCGTGACC |
| | sequence of | CCCGGCGAGCCCGCCAGCATCAGCTGCCGGAGCAGCCAGAG |
| | GPC3 | CCTGGTGCACAGCAACGGCAACACCTACCTGCAGTGGTACCT |
| | antibody VL | GCAGAAGCCCGGCCAGAGCCCCCAGCTGCTGATCTACAAGG |
| | | TGAGCAACCGGTTCAGCGGCGTGCCCGACCGGTTCAGCGGC |
| | | AGCGGCAGCGGCACCGACTTCACCCTGAAGATCAGCCGGGT |
| | | GGAGGCCGAGGACGTGGGCGTGTACTACTGCAGCCAGAGCA |
| | | TCTACGTGCCCTACACCTTCGGCCAGGGCACCAAGCTGGAGA |
| | | TCAAACGT |
| 40 | nucleic acid | GAGGTGCAATTGCTGGAGTCTGGGGGAGGCTTGGTACAGCC |
| | sequence of | TGGGGGGTCCCTGAGACTCTCCTGTGCAGCCTCCGGATTCAC |
| | GPRC5D | CTTTAGCAGTTATGCCATGAGCTGGGTCCGCCAGGCTCCAGG |
| | antibody VH | GAAGGGGCTGGAGTGGGTCTCAGCTATTAGTGGTAGTGGTGG |
| | | TAGCACATACTACGCAGACTCCGTGAAGGGCCGGTTCACCAT |
| | | CTCCAGAGACAATTCCAAGAACACGCTGTATCTGCAGATGAA |
| | | CAGCCTGAGAGCCGAGGACACGGCCGTATATTACTGTGCGCG |
| | | TGGTTGGCCATCTCCAGTTACTTTCGACTACTGGGGCCAAGG |
| | | AACCCTGGTCACCGTCTCGAGT |
| 41 | nucleic acid | GAAATCGTGTTAACGCAGTCTCCAGGCACCCTGTCTTTGTCT |
| | sequence of | CCAGGGGAAAGAGCCACCCTCTCTTGCAGGGCCAGTCAGAG |
| | GPRC5D | TGTTAGCAGCAGCTACTTAGCCTGGTACCAGCAGAAACCTGG |
| | antibody VL | CCAGGCTCCCAGGCTCCTCATCTATGGAGCATCCAGCAGGGC |
| | | CACTGGCATCCCAGACAGGTTCAGTGGCAGTGGATCCGGGA |
| | | CAGACTTCACTCTCACCATCAGCAGACTGGAGCCTGAAGATT |
| | | TTGCAGTGTATTACTGTCAGCAGTACAAATCTCATCCAATCAC |
| | | GTTCGGCCAGGGGACCAAAGTGGAAATCAAACGT |
| 42 | nucleic acid | GAGGTGCAATTGCTGGAGTCTGGGGGAGGCTTGGTACAGCC |
| | sequence of | TGGGGGGTCCCTGAGACTCTCCTGTGCAGCCTCCGGATTCAC |
| | BCMA | CTTTGGCGGTAATGCCATGTCCTGGGTCCGCCAGGCTCCAGG |
| | antibody VH | GAAGGGGCTGGAGTGGGTCTCAGCAATTAGTGGTAATGGTG |
| | | GTAGTACATTCTACGCAGACTCCGTGAAGGGCCGGTTCACCA |
| | | TCTCCAGAGACAATTCCAAGAACACGCTGTATCTGCAGATGA |
| | | ACAGCCTGAGAGCCGAGGACACGGCCGTATATTACTGTGCGA |
| | | AAGTTCGTCCATTCTGGGGTACTTTCGACTACTGGGGCCAAG |
| | | GAACCCTGGTCACCGTCTCGAGT |
| 43 | nucleic acid | GAAATCGTGTTAACGCAGTCTCCAGGCACCCTGTCTTTGTCT |
| | sequence of | CCAGGGGAAAGAGCCACCCTCTCTTGCAGGGCCAGTCAGAG |
| | BCMA | TGTTAGCAGCAGCTACTTAGCCTGGTACCAGCAGAAACCTGG |
| | antibody VL | CCAGGCTCCCAGGCTCCTCATCTATGGAGCATCCAGCAGGGC |
| | | CACTGGCATCCCAGACAGGTTCAGTGGCAGTGGATCCGGGA |
| | | CAGACTTCACTCTCACCATCAGCAGACTGGAGCCTGAAGATT |
| | | TTGCAGTGTATTACTGTCAGCAGTACTTCAACCCACCAGAATA |
| | | CACGTTCGGCCAGGGGACCAAAGTGGAAATCAAACGT |
| 44 | nucleic acid | CAGGTGCAGCTGGTGCAGAGCGGCGCCGAGGTGAAGAAGC |
| | sequence of | CCGGCGCCAGCGTGAAGGTGAGCTGCAAGGCCAGCGGCTAC |
| | NKG2A | ACCTTCACCAGCTACTGGATGAACTGGGTGCGGCAGGCCCCC |
| | antibody VH | GGCCAGGGCCTGGAGTGGATGGGCCGGATCGACCCCTACGA |
| | | CAGCGAGACCCACTACGCCCAGAAGCTGCAGGGCCGGGTGA |
| | | CCATGACCACCGACACCAGCACCAGCACCGCCTACATGGAG |
| | | CTGCGGAGCCTGCGGAGCGACGACACCGCCGTGTACTACTG |
| | | CGCCCGGGGCGGCTACGACTTCGACGTGGGCACCCTGTACTG |
| | | GTTCTTCGACGTGTGGGGCCAGGGCACCACCGTGACCGTGA |
| | | GCAGC |
| 45 | nucleic acid | GACATCCAGATGACCCAGAGCCCCAGCAGCCTGAGCGCCAG |
| | sequence of | CGTGGGCGACCGGGTGACCATCACCTGCCGGGCCAGCGAGA |
| | NKG2A | ACATCTACAGCTACCTGGCCTGGTACCAGCAGAAGCCCGGCA |
| | antibody VL | AGGCCCCCAAGCTGCTGATCTACAACGCCAAGACCCTGGCC |
| | | GAGGGCGTGCCCAGCCGGTTCAGCGGCAGCGGCAGCGGCAC |
| | | CGACTTCACCCTGACCATCAGCAGCCTGCAGCCCGAGGACTT |
| | | CGCCACCTACTACTGCCAGCACCACTACGGCACCCCCCGGAC |
| | | CTTCGGCGGCGGCACCAAGGTGGAGATCAAG |
| 46 | nucleic acid | GGAAGCGGAGCTACTAACTTCAGCCTGCTGAAGCAGGCTGG |
| | sequence of | AGACGTGGAGGAGAACCCTGGACCT |
| | P2A | |
| 47 | nucleic acid | GTGAAACAGACTTTGAATTTTGACCTTCTGAAGTTGGCAGGA |
| | sequence of | GACGTTGAGTCCAACCCTGGGCCC |
| | F2A | |
| 48 | nucleic acid | GGCTCCGGAGAGGGCAGAGGAAGTCTGCTAACATGCGGTGA |
| | sequence of | CGTCGAGGAGAATCCTGGACCT |
| | T2A | |
| 49 | gRNA-TRAC | AGAGTCTCTCAGCTGGTACA |
| 50 | gRNA-TRBC | GGCCTCGGCGCTGACGATCT |
| 51 | gRNA-B2M | GAGTAGCGCGAGCACAGCTA |
| 52 | amino acid | MALPVTALLLPLALLLHAARPEVQLVQSGAEVKKPGASVKV |
| | sequence of | SCKASGYTFSDYEMHWVRQAPGQGLEWMGAIHPGSGDTAY |
| | CAR1 | NQRFKGRVTITADKSTSTAYMELSSLRSEDTAVYYCARFYSY |
| | | AYWGQGTLVTVSAGGGGSGGGGSGGGGSDIVMTQTPLSLP |
| | | VTPGEPASISCRSSQSLVHSNGNTYLQWYLQKPGQSPQLLIY |
| | | KVSNRFSGVPDRFSGSGSGTDFTLKISRVEAEDVGVYYCSQSI |
| | | YVPYTFGQGTKLEIKRTTTPAPRPPTPAPTIASQPLSLRPEACR |
| | | PAAGGAVHTRGLDFACDIYIWAPLAGTCGVLLLSLVITLYCK |
| | | RGRKKLLYIFKQPFMRPVQTTQEEDGCSCRFPEEEEGGCELR |
| | | VKFSRSADAPAYQQGQNQLYNELNLGRREEYDVLDKRRGR |
| | | DPEMGGKPQRRKNPQEGLYNELQKDKMAEAYSEIGMKGER |
| | | RRGKGHDGLYQGLSTATKDTYDALHMQALPPR |
| 53 | amino acid | MALPVTALLLPLALLLHAARPEVQLLESGGGLVQPGGSLRLS |
| | sequence of | CAASGFTFSSYAMSWVRQAPGKGLEWVSAISGSGGSTYYAD |
| | CAR2 | SVKGRFTISRDNSKNTLYLQMNSLRAEDTAVYYCARGWPSP |
| | | VTFDYWGQGTLVTVSSGGGGSGGGGGGGGSEIVLTQSPGT |
| | | LSLSPGERATLSCRASQSVSSSYLAWYQQKPGQAPRLLIYGA |
| | | SSRATGIPDRFSGSGSGTDFTLTISRLEPEDFAVYYCQQYKSH |
| | | PITFGQGTKVEIKRTTTPAPRPPTPAPTIASQPLSLRPEACRPA |
| | | AGGAVHTRGLDFACDIYIWAPLAGTCGVLLLSLVITLYCKRG |
| | | RKKLLYIFKQPFMRPVQTTQEEDGCSCRFPEEEEGGCELRVK |
| | | FSRSADAPAYQQGQNQLYNELNLGRREEYDVLDKRRGRDPE |
| | | MGGKPQRRKNPQEGLYNELQKDKMAEAYSEIGMKGERRRG |
| | | KGHDGLYQGLSTATKDTYDALHMQALPPR |
| 54 | amino acid | MDMRVPAQLLGLLLLWLSGARCDIVMTQTPLSLPVTPGEPA |
| | sequence of | SISCRSSQSLVHSNGNTYLQWYLQKPGQSPQLLIYKVSNRFS |
| | recombinant | GVPDRFSGSGSGTDFTLKISRVEAEDVGVYYCSQSIYVPYTF |
| | TCR1 | GQGTKLEIKREDLNKVFPPEVAVFEPSEAEISHTQKATLVCL |
| | | ATGFFPDHVELSWWVNGKEVHSGVSTDPQPLKEQPALNDSR |
| | | YCLSSRLRVSATFWQNPRNHFRCQVQFYGLSENDEWTQDRA |
| | | KPVTQIVSAEAWGRADCGFTSVSYQQGVLSATILYEILLGKA |
| | | TLYAVLVSALVLMAMVKRKDFGSGATNFSLLKQAGDVEEN |
| | | PGPMEFGLSWVFLVALFRGVQCEVQLVQSGAEVKKPGASV |
| | | KVSCKASGYTFSDYEMHWVRQAPGQGLEWMGAIHPGSGDT |
| | | AYNQRFKGRVTITADKSTSTAYMELSSLRSEDTAVYYCARF |
| | | YSYAYWGQGTLVTVSAIQNPDPAVYQLRDSKSSDKSVCLFT |
| | | DFDSQTNVSQSKDSDVYITDKTVLDMRSMDFKSNSAVAWS |
| | | NKSDFACANAFNNSIIPEDTFFPSPESSCDVKLVEKSFETDTN |
| | | LNFQNLLVIVLRILLLKVAGFNLLMTLRLWSS |
| 55 | amino acid | MDMRVPAQLLGLLLLWLSGARCEIVLTQSPGTLSLSPGERAT |
| | sequence of | LSCRASQSVSSSYLAWYQQKPGQAPRLLIYGASSRATGIPDR |
| | recombinant | FSGSGSGTDFTLTISRLEPEDFAVYYCQQYKSHPITFGQGTKV |
| | TCR2 | EIKREDLNKVFPPEVAVFEPSEAEISHTQKATLVCLATGFFPD |
| | | HVELSWWVNGKEVHSGVSTDPQPLKEQPALNDSRYCLSSRL |
| | | RVSATFWQNPRNHFRCQVQFYGLSENDEWTQDRAKPVTQIV |
| | | SAEAWGRADCGFTSVSYQQGVLSATILYEILLGKATLYAVL |
| | | VSALVLMAMVKRKDFGSGATNFSLLKQAGDVEENPGPMEF |
| | | GLSWVFLVALFRGVQCEVQLLESGGGLVQPGGSLRLSCAAS |
| | | GFTFSSYAMSWVRQAPGKGLEWVSAISGSGGSTYYADSVKG |
| | | RFTISRDNSKNTLYLQMNSLRAEDTAVYYCARGWPSPVTFD |
| | | YWGQGTLVTVSSIQNPDPAVYQLRDSKSSDKSVCLFTDFDS |
| | | QTNVSQSKDSDVYITDKTVLDMRSMDFKSNSAVAWSNKSD |
| | | FACANAFNNSIIPEDTFFPSPESSCDVKLVEKSFETDTNLNFQ |
| | | NLLVIVLRILLLKVAGFNLLMTLRLWSS |
| 56 | amino acid | MDMRVPAQLLGLLLLWLSGARCEIVLTQSPGTLSLSPGERAT |
| | sequence of | LSCRASQSVSSSYLAWYQQKPGQAPRLLIYGASSRATGIPDR |
| | recombinant | FSGSGSGTDFTLTISRLEPEDFAVYYCQQYFNPPEYTFGQGTK |
| | TCR3 | VEIKREDLNKVFPPEVAVFEPSEAEISHTQKATLVCLATGFFP |
| | | DHVELSWWVNGKEVHSGVSTDPQPLKEQPALNDSRYCLSSR |
| | | LRVSATFWQNPRNHFRCQVQFYGLSENDEWTQDRAKPVTQI |
| | | VSAEAWGRADCGFTSVSYQQGVLSATILYEILLGKATLYAV |
| | | LVSALVLMAMVKRKDFGSGATNFSLLKQAGDVEENPGPME |
| | | FGLSWVFLVALFRGVQCEVQLLESGGGLVQPGGSLRLSCAA |
| | | SGFTFGGNAMSWVRQAPGKGLEWVSAISGNGGSTFYADSV |
| | | KGRFTISRDNSKNTLYLQMNSLRAEDTAVYYCAKVRPFWGT |
| | | FDYWGQGTLVTVSSIQNPDPAVYQLRDSKSSDKSVCLFTDF |
| | | DSQTNVSQSKDSDVYITDKTVLDMRSMDFKSNSAVAWSNK |
| | | SDFACANAFNNSIIPEDTFFPSPESSCDVKLVEKSFETDTNLNF |
| | | QNLLVIVLRILLLKVAGFNLLMTLRLWSS |
| 57 | amino acid | MDMRVPAQLLGLLLLWLSGARCDIQMTQSPSSLSASVGDRV |
| | sequence of | TITCRASENIYSYLAWYQQKPGKAPKLLIYNAKTLAEGVPSR |
| | recombinant | FSGSGSGTDFTLTISSLQPEDFATYYCQHHYGTPRTFGGGTK |
| | TCR4 | VEIKEDLNKVFPPEVAVFEPSEAEISHTQKATLVCLATGFFPD |
| | | HVELSWWVNGKEVHSGVSTDPQPLKEQPALNDSRYCLSSRL |
| | | RVSATFWQNPRNHFRCQVQFYGLSENDEWTQDRAKPVTQIV |
| | | SAEAWGRADCGFTSVSYQQGVLSATILYEILLGKATLYAVL |
| | | VSALVLMAMVKRKDFGSGATNFSLLKQAGDVEENPGPMEF |
| | | GLSWVFLVALFRGVQCQVQLVQSGAEVKKPGASVKVSCKA |
| | | SGYTFTSYWMNWVRQAPGQGLEWMGRIDPYDSETHYAQK |
| | | LQGRVTMTTDTSTSTAYMELRSLRSDDTAVYYCARGGYDF |
| | | DVGTLYWFFDVWGQGTTVTVSSIQNPDPAVYQLRDSKSSDK |
| | | SVCLFTDFDSQTNVSQSKDSDVYITDKTVLDMRSMDFKSNS |
| | | AVAWSNKSDFACANAFNNSIIPEDTFFPSPESSCDVKLVEKSF |
| | | ETDTNLNFQNLLVIVLRILLLKVAGFNLLMTLRLWSS |
| 58 | amino acid | MLLLVTSLLLCELPHPAFLLIPDIVMTQTPLSLPVTPGEPASIS |
| | sequence of | CRSSQSLVHSNGNTYLQWYLQKPGQSPQLLIYKVSNRFSGVP |
| | recombinant | DRFSGSGSGTDFTLKISRVEAEDVGVYYCSQSIYVPYTFGQG |
| | TCR5 | TKLEIKRDLRNVTPPKVSLFEPSKAEIANKQKATLVCLARGFF |
| | | PDHVELSWWVNGKEVHSGVCTDPQAYKESNYSYCLSSRLR |
| | | VSATFWHNPRNHFRCQVQFHGLSEEDKWPEGSPKPVTQNIS |
| | | AEAWGRADCGITSASYQQGVLSATILYEILLGKATLYAVLVS |
| | | TLVVMAMVKRKNSGSGATNFSLLKQAGDVEENPGPMLLLV |
| | | TSLLLCELPHPAFLLIPEVQLVQSGAEVKKPGASVKVSCKAS |
| | | GYTFSDYEMHWVRQAPGQGLEWMGAIHPGSGDTAYNQRF |
| | | KGRVTITADKSTSTAYMELSSLRSEDTAVYYCARFYSYAYW |
| | | GQGTLVTVSADIQNPEPAVYQLKDPRSQDSTLCLFTDFDSQI |
| | | NVPKTMESGTFITDKCVLDMKAMDSKSNGAIAWSNQTSFTC |
| | | QDIFKETNATYPSSDVPCDATLTEKSFETDMNLNFQNLLVIV |
| | | LRILLLKVAGFNLLMTLRLWSS |
| 59 | IL2 promoter | ACATTTTGACACCCCCATAATATTTTTCCAGAATTAACAGT |
| | | ATAAATTGCATCTCTTGTTCAAGAGTTCCCTATCACTCTCT |
| | | TTAATCACTACTCACAGTAACCTCAACTCCTG |

1. A chimeric polypeptide, wherein the polypeptide comprises an A chain and a B chain; the A chain comprises a first antigen-binding domain and a first constant region, and the B chain comprises a second antigen-binding domain and a second constant region:
(i) the first and/or second antigen-binding domain is an antibody heavy chain or heavy chain variable region, and is operably linked to a heavy chain signal peptide or a TCR subunit signal peptide;
(ii) the first and/or second antigen-binding domain is an antibody light chain or light chain variable region, and is operably linked to a light chain signal peptide or a TCR subunit signal peptide;
preferably, the first and/or second constant region comprises a transmembrane domain, optionally, the first and/or second constant region further comprises an intracellular domain.

2. (canceled)

3. The polypeptide according to claim 1, wherein the first and second constant regions are derived from natural and/or modified TCR subunit constant regions; preferably, the first and second constant regions are natural and/or modified TRAC and TRBC polypeptides, respectively; or the first and second constant regions are natural and/or modified TRGC and TRDC polypeptides, respectively;
preferably, the chimeric polypeptide can activate a CD3C polypeptide associated with the chimeric polypeptide after binding to the first antigen or the second antigen; preferably, the activation of the CD3ζ polypeptide can activate an immune effector cell expressing the chimeric polypeptide;
preferably, the first and second antigen-binding domains recognize the same or different antigens;
preferably, the first and second antigen-binding domains are heavy chain variable regions and light chain variable regions that recognize the same antigen, respectively.

4-6. (canceled)

7. The polypeptide according to claim 1, wherein the heavy chain signal peptide is selected from the heavy chain signal peptide of natural IgG, IgM, IgD, IgA or IgE, and the light chain signal peptide is selected from the light chain signal peptide of natural kappa (κ) or lambda (λ); and the TCR subunit signal peptide is selected from natural T cell receptor α chain signal peptide or β chain signal peptide, or the natural T cell receptor γ chain signal peptide or delta (δ) chain signal peptide;
preferably, the heavy chain signal peptide comprises any one of the following amino acid sequences: IgGsH1 (SEQ ID NO: 14), IgGsH2 (SEQ ID NO: 16), IgGsH3 (SEQ ID NO: 17) or IgGsH4 (SEQ ID NO: 18); the light chain signal peptide is selected from: IgGsL1 (SEQ ID NO: 9), IgGsL2 (SEQ ID NO: 11), IgGsL3 (SEQ ID NO: 12), and IgGsL4 (SEQ ID NO: 13); and the TCR subunit signal peptide comprises an amino acid sequence of SEQ ID NO: 1 or 3;
preferably, the heavy chain signal peptide and the light chain signal peptide are the following (i) or (ii):
(i) the heavy chain signal peptide is selected from: IgGsH1 (SEQ ID NO: 14), IgGsH2 (SEQ ID NO: 16) or IgGsH3 (SEQ ID NO: 17); and the light chain signal peptide is IgGsL1 (SEQ ID NO: 9);
(ii) the heavy chain signal peptide is IgGsH4 (SEQ ID NO: 18); and the light chain signal peptide is selected from: IgGsL3 (SEQ ID NO: 12) or IgGsL4 (SEQ ID NO: 13);
preferably, the antigen-binding domain is connected with the heavy chain signal peptide or the light chain signal peptide directly or through a linker.

8-10. (canceled)

11. The polypeptide according to claim 1, wherein the modified TCR subunit constant region comprises: a hydrophobic amino acid substitution and/or a cysteine substitution.

12. The polypeptide according to claim 1, wherein the nucleic acid sequence of the modified TCR subunit constant region comprises a mutation relative to the natural TCR subunit constant region, and preferably, the mutation is a synonymous mutation.

13. The polypeptide according to claim 1, wherein the first constant region comprises: the amino acid sequence of SEQ ID NO: 19, or the amino sequence obtained by hydrophobic amino acid substitution at positions 115, 118, and/or 119 of TRAC as shown in SEQ ID NO: 19; and/or the amino acid sequence obtained by substitution of the amino acid at position 47 of TRAC as shown in SEQ ID NO: 19 to cysteine; and the second constant region comprises: the amino acid sequence of SEQ ID NO: 21, or the amino acid sequence obtained by substitution of the amino acid at position 57 of TRBC as shown in SEQ ID NO: 21 to cysteine;
preferably, wherein its first constant region and second constant region are selected from (i) or (ii):
(i) the nucleic acid sequence encoding the first constant region comprises: the nucleic acid sequence of SEQ ID NO: 20, 25, 26, 27, 28, 29, 30 or 31, or the first constant region comprises the amino acid sequence encoded by the nucleic acid sequence of SEQ ID NO: 25, 26, 27, 28, 29, 30 or 31; and the nucleic acid sequence encoding the second constant region comprises: the nucleic acid sequence of SEQ ID NO: 22, 32, 33, or 34, or the second constant region comprises the amino acid sequence encoded by the nucleic acid sequence of SEQ ID NO: 32, 33, or 34;
(ii) the first constant region comprises the amino acid sequence of SEQ ID NO: 23; and the second constant region comprises the amino acid sequence of SEQ ID NO: 24;
preferably, its first constant region and second constant region are selected from (i), (ii) or (iii):
(i) the first constant region comprises the amino acid sequence of SEQ ID NO: 19; and the second constant region comprises the amino acid sequence of SEQ ID NO: 21;
(ii) the nucleic acid sequence encoding the first constant region comprises the nucleic acid sequence of SEQ ID NO: 20, 25, 26 or 29, or the first constant region comprises the amino acid sequence encoded by the nucleic acid sequence of SEQ ID NO: 20, 25, 26 or 29; and the nucleic acid sequence encoding the second constant region comprises the nucleic acid sequence of SEQ ID NO: 22 or 32, or the second constant region comprises the amino acid sequence encoded by the nucleic acid sequence of SEQ ID NO: 22 or 32;
(iii) the nucleic acid sequence encoding the first constant region comprises the nucleic acid sequence of SEQ ID NO: 27, 28, 30 or 31, or the first constant region comprises the amino acid sequence encoded by the nucleic acid sequence of SEQ ID NO: 27, 28, 30 or 31; and the nucleic acid sequence encoding the second constant region comprises the nucleic acid sequence of SEQ ID NO: 33 or 34, or the second constant region comprises the amino acid sequence encoded by the nucleic acid sequence of SEQ ID NO: 33 or 34;
preferably, the TRAC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 19; the TRBC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 21; the TRGC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 23; and the TRDC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 24.

14-16. (canceled)

17. The polypeptide according to claim 1, wherein the antigen is selected from tumor antigens, pathogen antigens or NK cell markers;
preferably, the antigen is selected from the group consisting of: GPC3, BCMA, GPRC5D, FAP, EGFR and a mutant thereof, ASGPR1, mesothelin, CD19, IL-13RA2, CLDN18.2, CLL1, CS1, NKG2A, TIGIT and CD94.

18. (canceled)

19. The polypeptide according to claim 1, wherein it is selected from any one of the following (i) to (v):
(i) the A chain comprises in sequence: a signal peptide as shown in SEQ ID NO: 14, an antibody heavy chain variable region, and a constant region as shown in SEQ ID NO: 19; and the B chain comprises in sequence: a signal peptide as shown in SEQ ID NO: 9, an antibody light chain variable region, and a constant region as shown in SEQ ID NO: 21;
(ii) the A chain comprises in sequence: a signal peptide as shown in SEQ ID NO: 14, an antibody heavy chain variable region, and the constant region of a nucleic acid sequence as shown in SEQ ID NO: 25, 26 or 29 or the amino acid sequence encoded thereof; and the B chain comprises in sequence: a signal peptide as shown in SEQ ID NO: 9, an antibody light chain variable region, and the constant region of a nucleic acid sequence of SEQ ID NO: 32 or the amino acid sequence encoded thereof;
(ii) the A chain comprises in sequence: a signal peptide as shown in SEQ ID NO: 14, an antibody heavy chain variable region, and the constant region of a nucleic acid sequence of SEQ ID NO: 27, 28, 30 or 31 or the amino acid sequence encoded thereof; and the B chain comprises in sequence: a signal peptide as shown in SEQ ID NO: 9, an antibody light chain variable region, and the constant region of a nucleic acid sequence of SEQ ID NO: 33 or 34 or the amino acid sequence encoded thereof;
(iii) the A chain comprises in sequence: a signal peptide as shown in SEQ ID NO: 1, an antibody heavy chain variable region, and a constant region as shown in SEQ ID NO: 19; and the B chain in sequence comprises: a signal peptide as shown in SEQ ID NO: 3, an antibody light chain variable region, and a constant region as shown in SEQ ID NO: 21;
(iv) the A chain comprises in sequence: a signal peptide as shown in SEQ ID NO: 1, an antibody heavy chain variable region, and the constant region of a nucleic acid sequence of SEQ ID NO: 25, 26 or 29 or the amino acid sequence encoded thereof; and the B chain comprises in sequence: a signal peptide as shown in SEQ ID NO: 3, an antibody light chain variable region, and the constant region of a nucleic acid sequence of SEQ ID NO: 32 or the amino acid sequence encoded thereof; or
(v) the A chain comprises in sequence: a signal peptide as shown in SEQ ID NO: 1, an antibody heavy chain variable region, and the constant region of a nucleic acid sequence of SEQ ID NO: 27, 28, 30 or 31 or the amino acid sequence encoded thereof; and the B chain comprises in sequence: a signal peptide as shown in SEQ ID NO: 3, an antibody light chain variable region, and the constant region of a nucleic acid sequence of SEQ ID NO: 33 or 34 or the amino acid sequence encoded thereof;
preferably, the polypeptide is selected from any one of the following (i) to (iv):
(i) a heavy chain variable region comprising the nucleic acid sequence of SEQ ID NO: 38 or the amino acid sequence encoded by SEQ ID NO: 38, and a light chain variable region comprising the nucleic acid sequence of SEQ ID NO: 39 or the amino acid sequence encoded by SEQ ID NO: 39;
(ii) a heavy chain variable region comprising the nucleic acid sequence of SEQ ID NO: 40 or the amino acid sequence encoded by SEQ ID NO: 40, and a light chain variable region comprising the nucleic acid sequence of SEQ ID NO: 41 or the amino acid sequence encoded by SEQ ID NO: 41;
(iii) a heavy chain variable region comprising the nucleic acid sequence of SEQ ID NO: 42 or the amino acid sequence encoded by SEQ ID NO: 42, and a light chain variable region comprising the nucleic acid sequence of SEQ ID NO: 43 or the amino acid sequence encoded by SEQ ID NO: 43; or
(iv) a heavy chain variable region comprising the nucleic acid sequence of SEQ ID NO: 44 or the amino acid sequence encoded by SEQ ID NO: 44, and a light chain variable region comprising the nucleic acid sequence of SEQ ID NO: 45 or the amino acid sequence encoded by SEQ ID NO: 45;
preferably, the polypeptide comprises an amino acid sequence or a fragment thereof having at least about 90%, at least about 95%, at least about 96%, at least about 97%, at least about 98%, at least about 99%, or at least about 100% homology or identity to an amino acid sequence of SEQ ID NO: 54, 55, 56 or 57.

20. (canceled)

21. (canceled)

22. An engineered cell comprising the polypeptide according to claim 1.

23. The cell according to claim 22, wherein the expression, activity and/or signaling of endogenous TCR subunits of the cell is reduced or inhibited;
preferably, the expression of endogenous TCR subunits is reduced or inhibited by using gene knockout technology and/or gene silencing technology selected from the group consisting of: TALE nuclease, meganuclease, zinc finger nuclease, CRISPR/Cas9, Argonaute, guided editing technology, meganuclease technology or a combination thereof; preferably, the nucleic acid molecule encoding the polypeptide does not comprise a nucleic acid sequence targeted by gene knockout technology and/or gene silencing technology;
preferably, the nucleic acid molecule encoding the polypeptide comprises a nucleic acid molecule that is no longer the target sequence targeted by gene knockout technology and/or gene silencing technology after synonymous mutation of the nucleotide(s);
preferably, the cell comprises a gRNA targeting wild-type TRAC and/or wild-type TRBC; preferably, the gRNA sequence comprised in the cell is as shown in SEQ ID NO: 49 and/or 50.

24-26. (canceled)

27. The cell according to claim 22, wherein the nucleic acid molecule comprised in the cell is selected from any one of the following (i) to (viii):
(i) a nucleic acid sequence comprising in sequence an antibody heavy chain signal peptide, an antibody VH, a natural and/or modified TRAC, a linker polypeptide, an antibody light chain signal peptide, an antibody VL, and a natural and/or modified TRBC;
(ii) a nucleic acid sequence comprising in sequence an antibody heavy chain signal peptide, an antibody VH, a natural and/or modified TRBC, a linker polypeptide, an antibody light chain signal peptide, an antibody VL, and a natural and/or modified TRAC;
(iii) a nucleic acid sequence comprising in sequence an antibody light chain signal peptide, an antibody VL, a natural and/or modified TRBC, a linker polypeptide, an antibody heavy chain signal peptide, an antibody VH, and a natural and/or modified TRAC;
(iv) a nucleic acid sequence comprising in sequence an antibody light chain signal peptide, an antibody VL, a natural and/or modified TRAC, a linker polypeptide, an antibody heavy chain signal peptide, an antibody VH, and a natural and/or modified TRBC;
(v) a nucleic acid sequence comprising in sequence TRAVs, an antibody VH, a natural and/or modified TRAC, a linker polypeptide, TRBVs, an antibody VL, and a natural and/or modified TRBC;
(vi) a nucleic acid sequence comprising in sequence TRBVs, an antibody VH, a natural and/or modified TRBC, a linker polypeptide, TRAVs, an antibody VL, and a natural and/or modified TRAC;
(vii) a nucleic acid sequence comprising in sequence TRBVs, an antibody VL, a natural and/or modified TRBC, a linker polypeptide, TRAVs, an antibody VH, and a natural and/or modified TRAC; or
(viii) a nucleic acid sequence comprising in sequence TRAVs, an antibody VL, a natural and/or modified TRAC, a linker polypeptide, TRBVs, an antibody VH, and a natural and/or modified TRBC;
preferably, the linker polypeptide is selected from: F2A, E2A, P2A or T2A.

28. (canceled)

29. (canceled)

30. The cell according to claim 22, wherein the cell is selected from the group consisting of: B cells, T cells, cytotoxic T lymphocytes (CTL), regulatory T cells, NK cells, natural killer T cells (NKT), human embryonic stem cells, and pluripotent stem cells;
preferably, the cell is an autologous or allogeneic cell;
preferably, the cell further expresses a secretory cytokine or a cytokine in membrane-bound form; preferably, the cell expresses IL12, IL15, IL18, IL21 and/or IL7.

31-35. (canceled)

36. A method for treating or preventing a tumor, including administering to a subject an effective amount of the immune effector cell claim 22;
preferably, the tumor is selected from the group consisting of: liver cancer, lung cancer, breast cancer, ovarian cancer, kidney cancer, thyroid cancer, gastric cancer, colorectal cancer, pancreatic cancer, multiple myeloma, and hematological malignancy;
preferably, the tumor is a GPC3-positive tumor, a BCMA-positive tumor, or a GPRC5D-positive tumor.

37-51. (canceled)

52. Engineered cells comprising chimeric polypeptide, wherein the chimeric polypeptide comprises:
(a) antigen-binding domain recognizing an antigen, and
(b) TCR subunit constant region obtained by synonymous mutations in nucleotide sequence;
wherein the expression, activity and/or signaling of endogenous TCR subunits of the engineered cell is reduced or inhibited, while the expression, activity, and/or signaling of the chimeric polypeptide is not reduced or inhibited;
preferably, the chimeric polypeptide can activate a CD3 molecule associated with the chimeric polypeptide after binding to the antigen.

53. The engineered cell according to claim 52, wherein the TCR subunit constant region comprises a hydrophobic amino acid substitution and/or a cysteine substitution.

54. The engineered cell according to claim 52, wherein the chimeric polypeptide comprises an A chain and a B chain; the A chain comprises a first antigen-binding domain and a first constant region, the B chain comprises a second antigen-binding domain and a second constant region; and the first and second constant regions are natural and/or modified TRAC and TRBC polypeptides, respectively;
preferably, the first and/or second antigen-binding domain is an antibody heavy chain or heavy chain variable region, and is operably linked to a heavy chain signal peptide or a TCR subunit signal peptide; or the first and/or second antigen-binding domain is an antibody light chain or light chain variable region, and is operably linked to a light chain signal peptide or a TCR subunit signal peptide;
preferably, the first constant region comprises: the amino acid sequence of SEQ ID NO: 19, or the amino sequence obtained by hydrophobic amino acid substitution at positions 115, 118, and/or 119 of the TRAC as shown in SEQ ID NO: 19; and/or the amino acid sequence obtained by substitution of the amino acid at position 47 of TRAC as shown in SEQ ID NO: 19 to cysteine; and the second constant region comprises: the amino acid sequence of SEQ ID NO: 21, or the amino acid sequence obtained by substitution of the amino acid at position 57 of TRBC as shown in SEQ ID NO: 21 to cysteine;
preferably, the TRAC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 19; the TRBC peptide comprises an amino acid sequence or a fragment thereof having at least about 80%, at least about 85%, at least about 90%, at least about 95%, at least about 96%, at least about 97%, at least about 98%, at least about 99%, or at least about 100% homology or identity to the amino acid sequence of SEQ ID NO: 21.

55. The engineered cell according to claim 54, wherein the heavy chain signal peptide is selected from the heavy chain signal peptide of natural IgG, IgM, IgD, IgA or IgE, and the light chain signal peptide is selected from the light chain signal peptide of natural kappa (κ) or lambda (λ); and the TCR subunit signal peptide is selected from natural T cell receptor α chain signal peptide or β chain signal peptide, or the natural T cell receptor γ chain signal peptide or delta (δ) chain signal peptide;
preferably, the heavy chain signal peptide comprises any one of the following amino acid sequences: IgGsH1 (SEQ ID NO: 14), IgGsH2 (SEQ ID NO: 16), IgGsH3 (SEQ ID NO: 17) or IgGsH4 (SEQ ID NO: 18); the light chain signal peptide is selected from: IgGsL1 (SEQ ID NO: 9), IgGsL2 (SEQ ID NO: 11), IgGsL3 (SEQ ID NO: 12), and IgGsL4 (SEQ ID NO: 13); and the TCR subunit signal peptide comprises an amino acid sequence of SEQ ID NO: 1 or 3;
preferably, the heavy chain signal peptide and the light chain signal peptide are the following (i) or (ii):
(i) the heavy chain signal peptide is selected from: IgGsH1 (SEQ ID NO: 14), IgGsH2 (SEQ ID NO: 16) or IgGsH3 (SEQ ID NO: 17); and the light chain signal peptide is IgGsL1 (SEQ ID NO: 9);
(ii) the heavy chain signal peptide is IgGsH4 (SEQ ID NO: 18); and the light chain signal peptide is selected from: IgGsL3 (SEQ ID NO: 12) or IgGsL4 (SEQ ID NO: 13);
preferably, the antigen-binding domain is connected with the heavy chain signal peptide or the light chain signal peptide directly or through a linker.

56. The engineered cell according to claim 54, wherein the TRAC peptide comprised in the chimeric polypeptide is connected with VH directly or through a linker, and the TRBC peptide comprised in the chimeric polypeptide is connected with VL directly or through a linker; or the TRAC peptide comprised in the chimeric polypeptide is connected with VL directly or through a linker, and the TRBC peptide comprised in the chimeric polypeptide is connected with VH directly or through a linker;
preferably, the sequences of the VH and VL comprised in the chimeric receptor respectively comprise sequences selected from one of the group consisting of: the sequences of SEQ ID NO: 38 and 39; or the sequences of SEQ ID NO: 40 and 41; or the sequences of SEQ ID NO: 42 and 43; or the sequences of SEQ ID NO: 44 and 45.

57. The engineered cell according to claim 52, the expression of endogenous TCR subunits is reduced or inhibited by using gene knockout technology and/or gene silencing technology selected from the group consisting of: TALE nuclease, meganuclease, zinc finger nuclease, CRISPR/Cas9, Argonaute, guided editing technology, meganuclease technology or a combination thereof;
preferably, the nucleic acid molecule encoding the chimeric polypeptide does not comprise a nucleic acid sequence targeted by gene knockout technology and/or gene silencing technology;
preferably, the nucleic acid molecule encoding the chimeric polypeptide comprises a nucleic acid molecule that is no longer the target sequence targeted by gene knockout technology and/or gene silencing technology after synonymous mutation of the nucleotide(s);
preferably, the engineered cell is used for treating or preventing a tumor;
preferably, the tumor is selected from the group consisting of: liver cancer, lung cancer, breast cancer, ovarian cancer, kidney cancer, thyroid cancer, gastric cancer, colorectal cancer, pancreatic cancer, multiple myeloma, and hematological malignancy;
preferably, the tumor is a GPC3-positive tumor, a BCMA-positive tumor, or a GPRC5D-positive tumor.

58. The engineered cell according to claim 52, wherein the engineered cell comprises gRNA(s), and the sequences of the gRNA(s) are as shown in SEQ ID NO: 49 and 50, respectively; or the sequences are shown in SEQ ID NO: 49, 50, and 51, respectively;
the cell comprises a gRNA targeting wild-type TRAC and/or wild-type TRBC; preferably, the gRNA sequence comprised in the cell is as shown in SEQ ID NO: 49 and/or 50;
preferably, the chimeric polypeptide comprises:
(i) the nucleic acid sequence encoding the TRAC comprises the nucleic acid sequence of SEQ ID NO: 25 or 26, or the TRAC comprises the amino acid sequence encoded by the nucleic acid sequence of SEQ ID NO: 25 or 26; and the nucleic acid sequence encoding the TRBC comprises the nucleic acid sequence of SEQ ID NO: 32, or the TRBC comprises the amino acid sequence encoded by the nucleic acid sequence of SEQ ID NO: 32;
(ii) the nucleic acid sequence encoding the TRAC comprises the nucleic acid sequence of SEQ ID NO: 27 or 28, or the TRAC comprises the amino acid sequence encoded by the nucleic acid sequence of SEQ ID NO: 27 or 28; and the nucleic acid sequence encoding the TRBC comprises the nucleic acid sequence of SEQ ID NO: 33, or the TRBC comprises the amino acid sequence encoded by the nucleic acid sequence of SEQ ID NO: 33.


SEQUENCE LISTING (59 sequences)

SEQ ID NO: 1 - protein, 27 aa, synthetic construct
MAMLLGASVLILWLQPDWVNSQQKNDD

SEQ ID NO: 2 - DNA, 81 bp, synthetic construct
  source 1..81: mol_type = other DNA
atggccatgctcctgggggcatcagtgctgattctgtggcttcagccagactgggtaaacagtcaacagaagaatgatgac

SEQ ID NO: 3 - protein, 21 aa, synthetic construct
MGTSLLCWMALCLLGADHADT

SEQ ID NO: 4 - DNA, 63 bp, synthetic construct
  source 1..63: mol_type = other DNA
atgggcaccagcctcctctgctggatggccctgtgtctcctgggggcagatcacgcagatact

SEQ ID NO: 5 - protein, 17 aa, synthetic construct
MWLQSLLLLGTVACSIS

SEQ ID NO: 6 - DNA, 51 bp, synthetic construct
  source 1..51: mol_type = other DNA
atgtggctgcagagcctgctgctcttgggcactgtggcctgcagcatctct

SEQ ID NO: 7 - protein, 22 aa, synthetic construct
MLLLVTSLLLCELPHPAFLLIP

SEQ ID NO: 8 - DNA, 66 bp, synthetic construct
  source 1..66: mol_type = other DNA
atgcttctcctggtgacaagccttctgctctgtgagttaccacacccagcattcctcctgatccca

SEQ ID NO: 9 - protein, 22 aa, synthetic construct
MDMRVPAQLLGLLLLWLSGARC

SEQ ID NO: 10 - DNA, 66 bp, synthetic construct
  source 1..66: mol_type = other DNA
atggacatgagggtccctgctcagctcctggggctcctgctgctctggctctcaggtgccagatgt

SEQ ID NO: 11 - protein, 22 aa, synthetic construct
MKYLLPTAAAGLLLLAAQPAMA

SEQ ID NO: 12 - protein, 16 aa, synthetic construct
MVLQTQVFISLLLWIS

SEQ ID NO: 13 - protein, 16 aa, synthetic construct
MAWTPLFLFLLTCCPG

SEQ ID NO: 14 - protein, 19 aa, synthetic construct
MEFGLSWVFLVALFRGVQC

SEQ ID NO: 15 - DNA, 57 bp, synthetic construct
  source 1..57: mol_type = other DNA
atggagtttgggctgagctgggttttcctcgttgctctttttagaggtgtccagtgt

SEQ ID NO: 16 - protein, 19 aa, synthetic construct
MDWTWRFLFVVAAATGVQS

SEQ ID NO: 17 - protein, 26 aa, synthetic construct
MDLLHKNMKHLWFFLLLVAAPRWVLS

SEQ ID NO: 18 - protein, 16 aa, synthetic construct
MDWTWRILFLVAAATG

SEQ ID NO: 19 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 20 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 21 - protein, 177 aa, synthetic construct
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 22 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 23 - protein, 173 aa, synthetic construct
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 24 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 25 - DNA, 420 bp, synthetic construct
  source 1..420: mol_type = other DNA
atccagaaccctgaccctgccgtgtatcagctgagggattccaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 26 - DNA, 420 bp, synthetic construct
  source 1..420: mol_type = other DNA
atccagaaccctgaccctgccgtgtatcagctgagggattccaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgctggtgatcgtgctgcgaatcctcctcctgaaagtggccgggtttaatctgctcatgacgctgcggctgtggtccagc

SEQ ID NO: 27 - DNA, 420 bp, synthetic construct
  source 1..420: mol_type = other DNA
atccagaaccctgaccctgccgtgtatcagctgagggattccaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 28 - DNA, 420 bp, synthetic construct
  source 1..420: mol_type = other DNA
atccagaaccctgaccctgccgtgtatcagctgagggattccaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgctggtgatcgtgctgcgaatcctcctcctgaaagtggccgggtttaatctgctcatgacgctgcggctgtggtccagc

SEQ ID NO: 29 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgctggtgatcgtgctgcgaatcctcctcctgaaagtggccgggtttaatctgctcatgacgctgcggctgtggtccagc

SEQ ID NO: 30 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 31 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgcgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgctggtgatcgtgctgcgaatcctcctcctgaaagtggccgggtttaatctgctcatgacgctgcggctgtggtccagc

SEQ ID NO: 32 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagattgtctccgccgaagcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 33 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagattgtctccgccgaagcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 34 - DNA, 531 bp, synthetic construct
  source 1..531: mol_type = other DNA
gaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtctgcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 35 - DNA, 1620 bp, synthetic construct
  source 1..1620: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggctcgggcggtggtgggtcgggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 36 - DNA, 1764 bp, synthetic construct
  source 1..1764: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggctcgggcggtggtgggtcgggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 37 - DNA, 194 bp, synthetic construct
  source 1..194: mol_type = other DNA
ggaggaaaaactgtttcatacagaaggcgtggaggaaaaactgtttcatacagaaggcgtggaggaaaaactgtttcatacagaaggcgtcaattgtcctcgacggaggaaaaactgtttcatacagaaggcgtggaggaaaaactgtttcatacagaaggcgtggaggaaaaactgtttcatacagaaggcgt

SEQ ID NO: 38 - DNA, 345 bp, synthetic construct
  source 1..345: mol_type = other DNA
gaggtgcagctggtgcagagcggcgccgaggtgaagaagcccggcgccagcgtgaaggtgagctgcaaggccagcggctacaccttcagcgactacgagatgcactgggtgcggcaggcccccggccagggcctggagtggatgggcgccatccaccccggcagcggcgacaccgcctacaaccagcggttcaagggccgggtgaccatcaccgccgacaagagcaccagcaccgcctacatggagctgagcagcctgcggagcgaggacaccgccgtgtactactgcgcccggttctacagctacgcctactggggccagggcaccctggtgaccgtgagcgcc

SEQ ID NO: 39 - DNA, 339 bp, synthetic construct
  source 1..339: mol_type = other DNA
gacatcgtgatgacccagacccccctgagcctgcccgtgacccccggcgagcccgccagcatcagctgccggagcagccagagcctggtgcacagcaacggcaacacctacctgcagtggtacctgcagaagcccggccagagcccccagctgctgatctacaaggtgagcaaccggttcagcggcgtgcccgaccggttcagcggcagcggcagcggcaccgacttcaccctgaagatcagccgggtggaggccgaggacgtgggcgtgtactactgcagccagagcatctacgtgccctacaccttcggccagggcaccaagctggagatcaaacgt

SEQ ID NO: 40 - DNA, 357 bp, synthetic construct
  source 1..357: mol_type = other DNA
gaggtgcaattgctggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgcgtggttggccatctccagttactttcgactactggggccaaggaaccctggtcaccgtctcgagt

SEQ ID NO: 41 - DNA, 327 bp, synthetic construct
  source 1..327: mol_type = other DNA
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtacaaatctcatccaatcacgttcggccaggggaccaaagtggaaatcaaacgt

SEQ ID NO: 42 - DNA, 357 bp, synthetic construct
  source 1..357: mol_type = other DNA
gaggtgcaattgctggagtctgggggaggcttggtacagcctggggggtccctgagactctcctgtgcagcctccggattcacctttggcggtaatgccatgtcctgggtccgccaggctccagggaaggggctggagtgggtctcagcaattagtggtaatggtggtagtacattctacgcagactccgtgaagggccggttcaccatctccagagacaattccaagaacacgctgtatctgcagatgaacagcctgagagccgaggacacggccgtatattactgtgcgaaagttcgtccattctggggtactttcgactactggggccaaggaaccctggtcaccgtctcgagt

SEQ ID NO: 43 - DNA, 330 bp, synthetic construct
  source 1..330: mol_type = other DNA
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtacttcaacccaccagaatacacgttcggccaggggaccaaagtggaaatcaaacgt

SEQ ID NO: 44 - DNA, 375 bp, synthetic construct
  source 1..375: mol_type = other DNA
caggtgcagctggtgcagagcggcgccgaggtgaagaagcccggcgccagcgtgaaggtgagctgcaaggccagcggctacaccttcaccagctactggatgaactgggtgcggcaggcccccggccagggcctggagtggatgggccggatcgacccctacgacagcgagacccactacgcccagaagctgcagggccgggtgaccatgaccaccgacaccagcaccagcaccgcctacatggagctgcggagcctgcggagcgacgacaccgccgtgtactactgcgcccggggcggctacgacttcgacgtgggcaccctgtactggttcttcgacgtgtggggccagggcaccaccgtgaccgtgagcagc

SEQ ID NO: 45 - DNA, 321 bp, synthetic construct
  source 1..321: mol_type = other DNA
gacatccagatgacccagagccccagcagcctgagcgccagcgtgggcgaccgggtgaccatcacctgccgggccagcgagaacatctacagctacctggcctggtaccagcagaagcccggcaaggcccccaagctgctgatctacaacgccaagaccctggccgagggcgtgcccagccggttcagcggcagcggcagcggcaccgacttcaccctgaccatcagcagcctgcagcccgaggacttcgccacctactactgccagcaccactacggcaccccccggaccttcggcggcggcaccaaggtggagatcaag

SEQ ID NO: 46 - DNA, 66 bp, synthetic construct
  source 1..66: mol_type = other DNA
ggaagcggagctactaacttcagcctgctgaagcaggctggagacgtggaggagaaccctggacct

SEQ ID NO: 47 - DNA, 66 bp, synthetic construct
  source 1..66: mol_type = other DNA
gtgaaacagactttgaattttgaccttctgaagttggcaggagacgttgagtccaaccctgggccc

SEQ ID NO: 48 - DNA, 63 bp, synthetic construct
  source 1..63: mol_type = other DNA
ggctccggagagggcagaggaagtctgctaacatgcggtgacgtcgaggagaatcctggacct

SEQ ID NO: 49 - DNA, 20 bp, synthetic construct
  source 1..20: mol_type = other DNA
agagtctctcagctggtaca

SEQ ID NO: 50 - DNA, 20 bp, synthetic construct
  source 1..20: mol_type = other DNA
ggcctcggcgctgacgatct

SEQ ID NO: 51 - DNA, 20 bp, synthetic construct
  source 1..20: mol_type = other DNA
gagtagcgcgagcacagcta

SEQ ID NO: 52 - protein, 488 aa, synthetic construct
MALPVTALLLPLALLLHAARPEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAGGGGSGGGGSGGGGSDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 53 - protein, 488 aa, synthetic construct
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWPSPVTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYKSHPITFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 608 aa, synthetic construct
MDMRVPAQLLGLLLLWLSGARCDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKREDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEFGLSWVFLVALFRGVQCEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 55 - protein, 608 aa, synthetic construct
MDMRVPAQLLGLLLLWLSGARCEIVLTQSPGTLSLSPGERATLSCRASQSVSSSYLAWYQQKPGQAPRLLIYGASSRATGIPDRFSGSGSGTDFTLTISRLEPEDFAVYYCQQYKSHPITFGQGTKVEIKREDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEFGLSWVFLVALFRGVQCEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGWPSPVTFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 56 - protein, 609 aa, synthetic construct
MDMRVPAQLLGLLLLWLSGARCEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKREDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEFGLSWVFLVALFRGVQCEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 57 - protein, 612 aa, synthetic construct
MDMRVPAQLLGLLLLWLSGARCDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMEFGLSWVFLVALFRGVQCQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLLVIVLRILLLKVAGFNLLMTLRLWSS

SEQ ID NO: 58 - protein, 603 aa, synthetic construct
MLLLVTSLLLCELPHPAFLLIPDIVMTQTPLSLPVTPGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNSGSGATNFSLLKQAGDVEENPGPMLLLVTSLLLCELPHPAFLLIPEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSADIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 59 - DNA, 114 bp, synthetic construct
  source 1..114: mol_type = other DNA
acattttgacacccccataatatttttccagaattaacagtataaattgcatctcttgttcaagagttccctatcactctctttaatcactactcacagtaacctcaactcctg